(19) United States
(12) Patent Application Publication
(10) Pub. No.: US 2011/0123530 A1
(43) Pub. Date: May 26, 2011

(54) COMPOSITIONS AND METHODS FOR TREATING AND DIAGNOSING ASTHMA

(76) Inventors: Joseph R. Arron, San Mateo, CA (US); John V. Fahy, San Francisco, CA (US); Barmak Modrek, Durham, NC (US); Prescott Woodruff, San Francisco, CA (US)

(21) Appl. No.: 12/935,822

(22) PCT Filed: Mar. 31, 2009
(86) PCT No.: PCT/US09/39033

§ 371 Date: Jan. 26, 2011

Related U.S. Application Data
(60) Provisional application No. 61/072,572, filed on Mar. 31, 2008.
(60) Provisional application No. 61/041,480, filed on Apr. 1, 2008.
(60) Provisional application No. 61/128,383, filed on May 20, 2008.
(60) Provisional application No. 61/205,392, filed on Jan. 16, 2009.

(51) Int. Cl. A61K 39/395; C40B 30/00; G01N 33/68; C12Q 1/68; C12Q 1/06; A61K 38/17; A61P 11/06

(52) U.S. Cl. 424/134.1; 506/9; 435/7.92; 435/6.12; 435/39; 424/158.1; 514/1.7; 435/6.11

(57) ABSTRACT

Compositions, kits and methods for treating and diagnosing subtypes of asthma patients are provided. Also provided are methods for identifying effective asthma therapeutic agents and predicting responsiveness to asthma therapeutic agents.

### CROSS-REFERENCE TO RELATED APPLICATIONS

This application claims the benefit of U.S. provisional application Nos. 61/072,572 filed 31 Mar. 2008, 61/041,480 filed 1 Apr. 2008, 61/128,383 filed 20 May 2008, and 61/205,392 filed 16 Jan. 2009.

### FIELD

Compositions and methods for treating and diagnosing subtypes of asthma patients are provided. Also provided are methods for identifying effective asthma therapeutic agents and predicting responsiveness to asthma therapeutic agents.

### BACKGROUND

Asthma is traditionally thought to result from aeroallergen-induced inflammation driven by T-helper type 2 (Th2) processes and mediated by cytokines including interleukin (IL)-4, IL-5 and IL-13. IL-13 is a pleiotropic Th2 cytokine produced by activated T cells, basophils, eosinophils, and mast cells, and it has been strongly implicated in the pathogenesis of asthma in preclinical models [2]. Elevated levels of IL-13 have been detected in the airways of human asthma patients; however, this elevation is only observed in a subset of asthmatics [3-6]. Recent research has been directed at understanding how Th2 cytokines cause asthma-like pathology and physiology [49, 50].

While asthma is often characterized by eosinophilic infiltration of the airways, there is increasing evidence that there are other subtypes of the disease driven by alternative forms of inflammation [1, 39, 48]. For example, studies of the cellular components of airway inflammation in asthma provide evidence for distinct eosinophilic and non-eosinophilic phenotypes of asthma [1, 39, 48]. Whether the molecular mechanisms underlying these clinical and cellular phenotypes of asthma differ is unknown. The identification of and development of biomarkers for distinct molecular phenotypes of asthma would guide the direction of basic research and the clinical application of emerging asthma therapies that specifically target Th2 responses in the lung.

Periostin is a secreted protein associated with fibrosis whose expression is upregulated by recombinant IL-4 and IL-13 in bronchial epithelial cells [7, 8] and bronchial fibroblasts [9]. It is expressed at elevated levels in vivo in bronchial epithelial cells [8] and in the subepithelial bronchial layer [9] of human asthmatics as well as in a mouse model of asthma [10]. It is also expressed at elevated levels in the esophageal epithelium of patients with eosinophilic esophagitis in an IL-13 dependent manner [11]. Elevated periostin expression has been observed in several types of epithelial derived cancers [64-67], and elevated levels of soluble periostin have been observed in the serum of some cancer patients [64, 68-70].

Genome-wide expression microarray analyses of bronchial epithelial cells from 42 mild-to-moderate, steroid-naïve asthmatics and 28 healthy control subjects have been performed [8]. In those studies, three of the most differentially expressed epithelial genes between all asthmatics and all healthy controls were periostin, CLCA1, and serpinB2 [8]. Furthermore, those genes were significantly downregulated in bronchial epithelial cells of asthmatics after 7 days of inhaled corticosteroid (ICS) treatment [8]. All three of those genes are induced in bronchial epithelial cells by recombinant IL-13 treatment in vitro and their expression is markedly attenuated by addition of corticosteroids to the cell culture medium [8].

To date, such genome-wide expression analyses have not identified genetic biomarkers that are prognostic or predictive of therapeutic response to treatment for individual asthma patients, nor have they identified genetic biomarkers that distinguish subtypes of asthmatic patients. In addition, no reliable nongenetic biomarkers with broad clinical applicability for prognostic or predictive responses to therapeutic treatment, or diagnostic of subtypes of asthma, have been identified. Thus, as asthma patients seek treatment, there is considerable trial and error involved in the search for therapeutic agent(s) effective for a particular patient. Such trial and error often involves considerable risk and discomfort to the patient in order to find the most effective therapy.

Thus, there is a need for more effective means for determining which patients will respond to which treatment and for incorporating such determinations into more effective treatment regimens for asthma patients.

The invention described herein meets the above-described needs and provides other benefits.

### SUMMARY

Using gene expression signatures in bronchial epithelium, we have defined distinct molecular subtypes of asthma. Surprisingly, supervised clustering of the data based on a set of genes whose expression was highly correlated to genes known to be upregulated by IL-4 or IL-13 stimulation revealed not one but two distinct clusters of asthma patients. Furthermore, analysis of these dichotomous subsets of asthmatics revealed significant associations between “IL-4/13 signature” status and serum total IgE levels, serum CEA levels, serum periostin levels, peripheral blood eosinophilia, (bronchoalveolar lavage) BAL eosinophilia, and responsiveness to inhaled corticosteroids (each p<0.05 by Wilcoxon rank sum test).

Accordingly, the present invention relates to methods of diagnosing a subpopulation of asthma patients comprising measuring the gene expression of any one or combination of genes selected from POSTN, CST1, CCL26, CLCA1, CST2, PRR4, SERPINB2, CEACAM5, iNOS, SERPINB4, CST4, PRB4, TPSD1, TPSG1, MFSD2, CPA3, GPR105, CDH26, GSN, C20RF32, TRACH2000196 (TMEM71), DNAJC12, RGS13, SLC18A2, SERPINB10, SH3RF2, FCER1B, RUNX2, PTGS1, and ALOX15. In one embodiment, the gene expression is measured of any one or combination of genes selected from the group of consisting of POSTN, CST1, CST2, CCL26, CLCA1, PRR4, PRB4, SERPINB2, CEACAM5, iNOS, SERPINB4, CST4, and SERPINB10. According to one embodiment, the gene expression is measured by microarray. According to another embodiment, the gene expression is measured by observing protein expression levels of an aforementioned gene. According to another embodiment, the gene expression is considered elevated when compared to a healthy control if the relative mRNA level of the gene of interest is greater than 2.5 of the level of a control gene mRNA. According to another embodiment, the relative mRNA level of the gene of interest is greater than 3 fold, 5 fold, 10 fold, 15 fold 25 fold or 30 fold compared to a healthy control gene expression level. According to one embodiment, the gene expression is measured by a method selected from the group consisting of a PCR method, a microarray method or a immunoassay method. In one embodiment, the microarray method comprises the use of a microarray chip having one or more nucleic acid molecules that can hybridize under stringent conditions to a nucleic acid molecule encoding a gene mentioned above or having one or more polypeptides (such as peptides or antibodies) that can bind to one or more of the proteins encoded by the genes mentioned above. In one embodiment, the PCR method is qPCR. According to one embodiment, the immunoassay method comprises the steps of binding an antibody to protein expressed from a gene mentioned above in a patient sample mentioned above and determining if the protein level from the patient sample is elevated. According to one embodiment, a control gene is a housekeeping gene selected from the group consisting of actin, GAPDH, GASB and GUSB.

The present invention provides a microarray chip comprising nucleic acid sequences encoding the following genes: POSTN, CST1, CST2, CCL26, CLCA1, PRR4, SERPINB2, CEACAM5, iNOS, SERPINB4, CST4, and SERPINB10 or fragments there of. The present invention provides a microarray chip comprising nucleic acid sequences encoding the following genes: POSTN, CST1, CCL26, CLCA1, CST2, PRR4, SERPINB2, CEACAM5, iNOS, SERPINB4, CST4, PRB4, TPSD1, TPSG1, MFSD2, CPA3, GPR105, CDH26, GSN, C20RF32, TRACH2000196 (TMEM71), DNAJC12, RGS13, SLC18A2, SERPINB10, SH3RF2, FCER1B, RUNX2, PTGS1, and ALOX1, or fragments thereof.

The present invention provides a subpopulation of asthma patients to be treated with the therapeutic agents of this invention, wherein the ratio of Muc5AC:MUC5B protein or mRNA levels in the airway epithelial cells of asthma patients is greater than 25.

The present invention also relates to methods of diagnosing a subpopulation of asthma patients by taking single or combinations of measurements of systemic biomarkers selected from serum CEA levels, serum IgE levels, serum periostin levels, peripheral blood eosinophil counts and eosinophil percentages in bronchoalveolar lavage fluid (BAL). Systemic biomarkers typically are nongenetic biomarkers and are typically measured in samples obtained by noninvasive procedures, for example, but not limited to, collection of blood or blood components, e.g., serum or plasma. According to one embodiment, greater than 100 IU/ml IgE levels and/or 0.14×10e9/L eosinophils is predictive of a patient population to be treated with the therapeutic agents of this invention.

The present invention relates to methods of treating asthma comprising administering a therapeutic agent to a patient expressing elevated levels of any one or combination of the genes selected from POSTN, CST1, CCL26, CLCA1, CST2, PRR4, SERPINB2, CEACAM5, iNOS, SERPINB4, CST4, PRB4, TPSD1, TPSG1, MFSD2, CPA3, GPR105, CDH26, GSN, C20RF32, TRACH2000196 (TMEM71), DNAJC12, RGS13, SLC18A2, SERPINB10, SH3RF2, FCER1B, RUNX2, PTGS1, ALOX15. According to one embodiment, the patient expresses elevated levels of any one or combination of genes selected from the group consisting of periostin, CST1, CST2, CCL26, CLCA1, PRR4, SerpinB2, CEACAM5, iNOS, PRB4, SerpinB4, SerpinB10 and CST4. According to one embodiment, the patient to be treated is a mild-to-moderate, steroid-naive (never treated with steroids) asthma patient. According to another embodiment, the patient to be treated is a moderate-to-severe, steroid-resistant (non-responsive to steroids) asthma patient. Such patients are treated with a therapeutically effective amount of a therapeutic agent. In one embodiment, the patient has asthma induced by the TH2 pathway.

According to one embodiment, the therapeutic agent is an anti-IL13/IL4 pathway inhibitor. According to another embodiment, the therapeutic agent targets the TH2 induced asthma pathway. Exemplary targets include, but are not limited to, cytokines or ligands such as: IL-9, IL-5, IL-13, IL-4, OX40L, TSLP, IL-25, IL-33 and IgE; and receptors such as: IL-9 receptor, IL-5 receptor, IL-4receptor alpha, IL-13receptoralpha1 and IL-13receptoralpha2, OX40, TSLP-R, IL-7Ralpha (a co-receptor for TSLP), IL17RB (receptor for IL-25), ST2 (receptor for IL-33), CCR3, CCR4, CRTH2, FcepsilonRI and FcepsilonRII/CD23 (receptors for IgE). Accordingly, a therapeutic agent according to this invention includes an agent that can bind to the target above, such as a polypeptide(s) (e.g., an antibody, an immunoadhesin or a peptibody), an aptamer or a small molecule.

According to one embodiment, the therapeutic agent is an anti-IL13 antibody. According to another embodiment, the anti-IL-13 antibody comprises a VH sequence comprising SEQ ID NO: 193 and a VL sequence comprising SEQ ID NO:194. According to another embodiment, the anti-IL13 antibody comprises: (a) an HVR-L1 comprising amino acid sequence RASKSVDSYGNSFMH (SEQ ID NO:195); (b) an HVR-L2 comprising amino acid sequence LASNLES (SEQ ID NO:196); (c) an HVR-L3 comprising amino acid sequence QQNNEDPRT (SEQ ID NO: 197); (d) an HVR-H1 comprising amino acid sequence AYSVN (SEQ ID NO:198); (e) an HVR-H2 comprising amino acid sequence MIWGDGKIVYNSALKS (SEQ ID NO: 199); and (f) an HVR-H3 comprising amino acid sequence DGYYPYAMDN (SEQ ID NO: 200). According to another embodiment, the therapeutic agent is an anti-OX40 ligand (OX40L) antibody. According to another embodiment the therapeutic agent is an anti-IL13/anti-IL4 bispecific antibody. According to another embodiment, the therapeutic agent is an anti-IgE antibody. According to another embodiment, the therapeutic agent is an antibody directed against the membrane proximal M1′ region of surface expressed IgE on B cells. According to another embodiment, the therapeutic agent is an inhaled corticosteroid. In certain embodiments, the inhaled corticosteroid is selected from beclomethasone dipropionate, budesonide, flunisolide, fluticasone propionate, mometasone, and triamcinolone acetonide.

According to one embodiment, the anti-OX40L antibody comprises: (a) an HVR-L1 comprising sequence RSSQSPVHSNGNTYLH (SEQ ID NO:201); (b) an HVR-L2 comprising sequence KVSNRFS (SEQ ID NO: 202); (c) an HVR-L3 comprising sequence SQSTHIPWT (SEQ ID NO: 203); (d) an HVR-H1 comprising sequence SYWMH (SEQ ID NO: 204); (e) an HVR-H2 comprising sequence EIDPSNGRTNYNEKFKS (SEQ ID NO: 205); and (f) an HVR-H3 comprising sequence ERSPRYFDV (SEQ ID NO:206). According to another embodiment, the anti-OX40L antibody comprises: (a) an HVR-L1 comprising sequence RSSQSIVHGNGNTYLE (SEQ ID NO:207); (b) an HVR-L2 comprising sequence RVSNRFS (SEQ ID NO:208); (c) an HVR-L3 comprising sequence FQGSHVPYT (SEQ ID NO:209); (d) an HVR-H1 comprising sequence SYWLN (SEQ ID NO:210); (e) an HVR-H2 comprising sequence MIDPSDSETHYNQVFKD (SEQ ID NO:211); and (f) an HVR-H3 comprising sequence GRGNFYGGSHAMEY (SEQ ID NO:212). According to another embodiment, the anti-OX40L antibody comprises (a) an HVR-H1 comprising sequence SYTMH (SEQ ID NO:215), SYAMS (SEQ ID NO:216), NFGMH (SEQ ID NO:217), or NYGMH (SEQ ID NO:218), (b) an HVR-H2 comprising sequence IISGSGGFTYYADSVKG (SEQ ID NO:219), AIWYDGHDKYYSYYVKG (SEQ ID NO:220), AIWYDGHDKYYAYYVKG (SEQ ID NO:221), VIWYDGSNKYYVDSVKG (SEQ ID NO:222), or VIWNDGSNKYYVDSVKG (SEQ ID NO:223), (c) an HVR-H3 comprising sequence DSSSWYRYFDY (SEQ ID NO:224), DRLVAPGTFDY (SEQ ID NO:225), KNWSFDF (SEQ ID NO:226), or DRMGIYYYGMDV (SEQ ID NO:227), (d) an HVR-L1 comprising sequence RASQGISSWLA (SEQ ID NO:228), RASQSVSSSYLA (SEQ ID NO:229), RASQSVSSNYLA (SEQ ID NO:230), RASQGVSRYLA (SEQ ID NO:231), or RASQSVSSYLA (SEQ ID NO:232), (e) an HVR-L2 comprising sequence GASSRAT (SEQ ID NO:233), AASSLQS (SEQ ID NO:234), MPPVWKV (SEQ ID NO:235), DASNRAT (SEQ ID NO:236), or LHPLCKV (SEQ ID NO:237); and (f) an HVR-L3 comprising sequence NSLIVTLT (SEQ ID NO:238), QQYNSYPYT (SEQ ID NO:239), QQYGSSFT (SEQ ID NO:240), QQRSNWQYT (SEQ ID NO:241), QQRSNWT (SEQ ID NO:242), or NSIIVSLT (SEQ ID NO:243), wherein the anti-OX40L antibody binds OX40L. According to one embodiment, the anti-IgE antibody comprises a VL sequence comprising SEQ ID NO:213 and a VH sequence comprising SEQ ID NO:214. According to another embodiment, the anti-IgE antibody comprises: (a) an HVR-L1 comprising sequence RSSQSLVHNNANTYLH (SEQ ID NO:244) (b) an HVR-L2 comprising sequence KVSNRFS (SEQ ID NO: 245); (c) an HVR-L3 comprising sequence SQNTLVPWT (SEQ ID NO: 246); (d) an HVR-H1 comprising sequence GFTFSDYGIA (SEQ ID NO: 247); (e) an HVR-H2 comprising sequence AFISDLAYTIYYADTVTG (SEQ ID NO: 248); and (f) an HVR-H3 comprising sequence ARDNWDAMDY (SEQ ID NO:249). According to one embodiment, the anti-IgE antibody comprises a VH sequence comprising SEQ ID NO:250 and a VL sequence comprising SEQ ID NO:251. According to one embodiment, the anti-IgE antibody comprises a VH sequence comprising SEQ ID NO:252 and a VL sequence comprising SEQ ID NO:253. According to another embodiment, the anti-IgE antibody comprises: (a) an HVR-L1 comprising sequence RSSQDISNSLN (SEQ ID NO:254) (b) an HVR-L2 comprising sequence STSRLHS (SEQ ID NO: 255); (c) an HVR-L3 comprising sequence QQGHTLPWT (SEQ ID NO: 256); (d) an HVR-H1 comprising sequence GYTFTDYYMM (SEQ ID NO: 257); (e) an HVR-H2 comprising sequence GDNIDPNNYDTSYNQKFKG (SEQ ID NO: 258); and (f) an HVR-H3 comprising sequence ASKAY (SEQ ID NO:259). According to another embodiment, the anti-IgE antibody comprises: (a) an HVR-L1 comprising sequence RSSQDISNALN (SEQ ID NO:260) (b) an HVR-L2 comprising sequence STSRLHS (SEQ ID NO: 255); (c) an HVR-L3 comprising sequence QQGHTLPWT (SEQ ID NO: 256); (d) an HVR-H1 comprising sequence GYTFTDYYMM (SEQ ID NO: 257); (e) an HVR-H2 comprising sequence GDNIDPNNYDTSYNQKFKG (SEQ ID NO: 258); and (f) an HVR-H3 comprising sequence ASKAY (SEQ ID NO:259). According to another embodiment, the anti-IgE antibody comprises: (a) an HVR-L1 comprising sequence RSSQDISNALN (SEQ ID NO:260) (b) an HVR-L2 comprising sequence STSRLHS (SEQ ID NO: 255); (c) an HVR-L3 comprising sequence QQGHTLPWT (SEQ ID NO: 256); (d) an HVR-H1 comprising sequence GYTFTDYYIM (SEQ ID NO: 261); (e) an HVR-H2 comprising sequence GDNIDPNNYDTSYNQKFKG (SEQ ID NO: 258); and (f) an HVR-H3 comprising sequence ASKAY (SEQ ID NO:259).

According to one embodiment, the patient has asthma that does not involve the TH2 pathway (non-TH2 asthma). In one embodiment, the therapeutic agent targets non-TH2 asthma. According to one embodiment, the therapeutic agent is an IL-17 pathway inhibitor.

In one embodiment, the therapeutic agent is anti-IL-17 antibody. In one embodiment, the therapeutic agent is an antibody cross-reactive with both IL-17A and IL-17F. In one embodiment, the therapeutic agent is a bispecific antibody capable of binding both IL-17A and IL-17F. In one embodiment, the therapeutic agent is an anti-IL-17A/F antibody.

The present invention provides a kit for diagnosing an asthma subtype in a patient comprising (1) one or more nucleic acid molecules that hybridize with a gene, wherein the gene is selected from the group of consisting of POSTN, CST1, CST2, CCL26, CLCA1, PRR4, PRB4, SERPINB2, CEACAM5, iNOS, SERPINB4, CST4, and SERPINB10 and (2) instructions for measuring the expression levels of the gene from an asthma patient sample, wherein the elevated expression levels of any one, combination or all of said genes is indicative of the asthma subtype. According to one embodiment, the kit further comprises a gene selected from the group consisting of: PRB4, TPSD1, TPSG1, MFSD2, CPA3, GPR105, CDH26, GSN, C20RF32, TRACH2000196 (TMEM71), DNAJC12, RGS13, SLC18A2, SH3RF2, FCER1B, RUNX2, PTGS1, and ALOX15. In one further embodiment, the gene expression level is measured by assaying for mRNA levels. In another further embodiment, the assay comprises a PCR method or the use of a microarray chip. In yet a further embodiment, the PCR method is qPCR. In one embodiment, the mRNA levels of the gene of interest relative to a control gene mRNA level greater than 2.5 fold is indicative of the asthma subtype.

The invention provides a kit for diagnosing an asthma subtype in a patient comprising (1) one or more protein molecules that bind to a protein selected from the group of consisting of POSTN, CST1, CST2, CCL26, CLCA1, PRR4, PRB4, SERPINB2, CEACAM5, iNOS, SERPINB4, CST4, and SERPINB10 and (2) instructions for measuring the expression levels of the protein from a patient sample, wherein the elevated expression levels of any one, combination or all of said proteins is indicative of the asthma subtype. In one embodiment, the kit further comprises a protein molecule that binds to a protein selected from the group consisting of: PRB4, TPSD1, TPSG1, MFSD2, CPA3, GPR105, CDH26, GSN, C20RF32, TRACH2000196 (TMEM71), DNAJC12, RGS13, SLC18A2, SH3RF2, FCER1B, RUNX2, PTGS1, and ALOX15. In one embodiment the protein molecule is a antibody, a peptide or a peptibody. In a further embodiment, the kit comprises a microarray chip comprising the protein molecule(s).

The present invention provides a kit for diagnosing an asthma subtype in a patient comprising instructions for measuring any one of the biomarkers from a patient sample selected from the group consisting of: serum total IgE levels, serum CEA levels, serum periostin levels, peripheral blood eosinophils and bronchoalveolar lavage (BAL) eosinophils, wherein elevated levels of CEA, serum periostin, peripheral blood eosinophils and bronchoalveolar lavage (BAL) eosinophils. According to one embodiment, the kit provides instructions wherein an IgE level greater than 100 IU/ml is indicative of the asthma subtype. According to another embodiment, the kit provides instruction, wherein a peripheral blood eosinophil level greater than 0.14×10e9/L is indicative of the asthma subtype.

The present invention provides a kit for diagnosing an asthma subtype in a patient comprising instructions for measuring the ratio of Muc5AC:MUC5B mRNA or protein from a sample of an asthma patient, wherein a ratio greater than 25 is indicative of the asthma subtype. In one embodiment, the sample is obtained from an epithelial brushing. In another embodiment, the sample comprises airway epithelial cells. In one embodiment, the kit provides a nucleic acid molecule that hybridizes under stringent conditions with Muc5AC and a nucleic acid molecule that hybridizes under stringent conditions with MUC5B. In one embodiment, the kit provides a protein molecule that binds to Muc5AC and a protein molecule that binds to MUC5B. In one embodiment, the protein molecule is an antibody.

### BRIEF DESCRIPTION OF THE DRAWINGS

FIG. 1 shows gene expression levels in airway epithelium as described in Examples 1 and 2. (A) Relative expression levels of periostin (left panel), CLCA1 (middle panel), and serpinB2 (right panel) in healthy controls (N=27) and in asthmatics (N=42) are shown. Normalized fluorescence units are indicated on the left axis of each plot. (B) Two-way comparisons of expression levels of periostin and CLCA1 (left panel), periostin and serpinB2 (middle panel), and CLCA1 and serpinB2 (right panel) in 42 asthmatics are shown. Spearman's rank order correlation (ρ) and p-values are indicated in each panel. (C) Gene expression microarray analysis for healthy controls and asthmatics identifying expression levels of periostin and co-regulated genes; IL-4/13 signature high cluster (cluster 1); IL-4/13 signature low cluster (cluster 2); healthy controls. (D) Heatmap depicting unsupervised hierarchical clustering (Euclidean complete) of periostin, CLCA1, and serpinB2 expression levels in bronchial epithelium across all subjects at baseline. (E) Mean (±SEM) expression levels of IL-4, IL-5, and IL-13 in bronchial biopsy homogenates obtained contemporaneously with bronchial brushings from a subset of subjects depicted in FIGS. 1A-D (cluster 1: 18 “IL-13 high” asthmatics; cluster 2: 16 healthy controls and 14 “IL-13 low” asthmatics). Two-way correlations across all subjects between IL-4, IL-5, and IL-13 indicated at right (Spearman's rank order correlation, ρ, and p-values).

FIG. 2 shows gene families for serpins, cystatins, and PRRs, and expression levels of those genes as described in Example 3. (A) Serpins (top), cystatins (middle), and PRRs (bottom) genomic loci and organization as viewed at the University of California Santa Cruz genome browser available at http://genome.ucsc.edu. (B) Hierarchical clustering of all probes encoding cystatin and serpin genes as depicted in panel A. (C) Relative gene expression levels in airway epithelium of PRR4 (left panel), PRB4 (middle panel), and CEACAM5 (right panel) in healthy controls (N=27) and in asthmatics (N=42) are shown. Normalized fluorescence units are indicated on the left axis of each plot.

FIG. 3 shows microarray analysis of bronchial epithelial brushings at baseline and after one week of inhaled fluticasone propionate (ICS) treatment as described in Example 6. (A) Periostin expression; (B) PRR4 expression; (C) RUNX2 expression.

FIG. 4 shows a composite graph of serum IgE and peripheral blood eosinophils in asthmatic patients as described in Examples 7 and 9.

FIG. 5 shows various clinical features of IL-13 high and IL-13 low subphenotypes of asthma as described in Example 8. (A) Volume of air exhaled in the first second of a forced expiration (FEV₁), a measure of airway obstruction. (B) Improvement in FEV₁ after 4 puffs (360 μg) of albuterol (bronchodilator reversibility testing). (C) Provocative concentration of methacholine required to induce a 20% decline in FEV₁ (PC₂₀), a measure of airway hyper-responsiveness.

FIG. 6 shows various markers of allergy, eosinophilic inflammation and airway remodeling of IL-13-high and IL-13 low subphenotypes of asthma as described in Example 8. (A) Allergen skin prick test (SPT) results using a panel of 12 aeroallergens. (B) Serum IgE concentration. (C) Peripheral blood eosinophil count. (D) Eosinophils as a percentage of total bronchoalveolar lavage fluid (BAL) cells. (E) Stereologic measurement of reticular basement membrane (RBM) thickness on endobronchial biopsy, a measure of sub-epithelial fibrosis. (F) Ratio of MUC5AC to MUC5B expression in epithelial brushings as determined by qPCR.

FIG. 7 shows various clinical features of IL-13 high and IL-13 low subphenotypes of asthma as described in Example 8. (A) Percentage of subjects responding to specific aeroallergens as indicated along the bottom axis. “IL-13 low” asthma subphenotype; “IL-13 high” asthma subphenotype (*, p<0.05). (B) Number of positive SPT reactions vs. BAL eosinophil percentage; IL-13 asthma subphentoype as indicated (high, open squares; low, closed circles). (C) Number of positive SPT reactions vs. serum IgE; IL-13 asthma subphentoype as indicated (high, open squares; low, closed circles). (D) Number of positive SPT reactions vs. peripheral blood eosinophil count; IL-13 asthma subphentoype as indicated (high, open squares; low, closed circles). Spearman's rank order correlation (ρ) and p-values are indicated in each plot for B-D.

FIG. 8 shows airway epithelial mucin content and composition in subjects with IL-13 high and IL-13 low asthma subphenotypes and healthy controls as described in Example 8. (A) Volume of mucin per volume of epithelium, a measure of airway epithelial mucin content. (B) Expression of mucin MUC2 as determined by qPCR. (C) Expression of mucin MUC5AC as determined by qPCR. (D) Expression of mucin MUC5B as determined by qPCR.

FIG. 9 shows responses of subjects with IL-13 high and IL-13 low asthma subphenotypes to inhaled corticosteroids. (A) FEV₁ measured at baseline (week 0), after 4 and 8 weeks on daily fluticasone, and one week after the cessation of fluticasone (week 9). (*): see Table 5 for number of subjects in each group and p-values. (B) Heatmap depicting unsupervised hierarchical clustering of periostin, CLCA1, and serpinB2 (as in FIG. 1D) in bronchial epithelium of asthmatics one week after the initiation of either fluticasone (N=19) or placebo treatment (N=13). Cluster identification at baseline for individual subjects and treatment are indicated below heatmap. (cluster 1: “IL-13 high” asthmatics; cluster 2: “IL-13 low” asthmatics).

FIG. 10 shows alveolar macrophage gene expression in subjects with IL-13 high and IL-13 low subphenotypes of asthma as described in Example 8. Healthy controls (N=15); IL-13 low subphenotype of asthma (N=5); IL-13 high subphenotype of asthma (N=9) are indicated. The figure shows the mean (+SEM) expression levels of 15-lipoxygenase (ALOX15) and tumor necrosis factor-α (TNF-α) as determined by qPCR. (*): p<0.03.

FIG. 11 shows gene expression microarray analysis using 35 probes covering 28 genes of samples from healthy controls and asthmatics as described in Example 9.

FIG. 12 shows gene expression microarray analysis and qPCR analysis for periostin and CEACAM5 as described in Example 9. (A) Periostin expression in healthy controls, cluster 2 asthmatics (“IL-13 LOW”), and cluster 1 asthmatics (“IL-13 high”); (B) CEACAM5 expression in healthy controls, cluster 2 asthmatics (“IL-13 LOW”), and cluster 1 asthmatics (“IL-13 HIGH”); (C) a composite graph of CEACAM5 and periostin in “IL-13 high” asthmatics (squares) and “IL-13 low” asthmatics (circles); (D) Receiver operating characteristic (ROC) analysis of an optimized algorithm for qPCR-based expression levels of periostin and CEACAM5 showing sensitivity and specificity for healthy controls, “IL-13 high” asthmatics, and “IL-13 low” asthmatics.

FIG. 13 shows serum levels of serum proteins in asthmatics and in healthy controls as described in Example 9. (A) serum levels of IgE; (B) serum levels of periostin; (C) serum levels of CEA; (D) serum levels of YKL-40; (E) serum levels of IgE in asthmatics treated with inhaled corticosteroids (ICS) (+) or not (−); (F) serum levels of periostin in asthmatics treated with inhaled corticosteroids (ICS) (+) or not (−); (G) serum levels of CEA in asthmatics treated with inhaled corticosteroids (ICS) (+) or not (−); (H) serum levels of YKL-40 in asthmatics treated with inhaled corticosteroids (ICS) (+) or not (−); (I) composite graph of serum levels of periostin in asthmatics having <100 IU/ml serum IgE (<100) and asthmatics having ≧100 IU/ml serum IgE (≧100); (J) composite graph of serum levels of CEA in asthmatics having <100 IU/ml serum IgE (<100) and asthmatics having ≧100 IU/ml serum IgE (≧100); (K) composite graph of serum levels of YKL-40 in asthmatics having <100 IU/ml serum IgE (<100) and asthmatics having ≧100 IU/ml serum IgE (≧100); (L) composite graph of serum levels of periostin and CEA in asthmatics having <100 IU/ml serum IgE (circles) and asthmatics having ≧100 IU/ml serum IgE (squares).

### DETAILED DESCRIPTION

### Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, the widely utilized molecular cloning methodologies described in Sambrook et al., Molecular Cloning: A Laboratory Manual 2nd. edition (1989) Cold Spring Harbor Laboratory Press, Cold Spring Harbor, N.Y. As appropriate, procedures involving the use of commercially available kits and reagents are generally carried out in accordance with manufacturer defined protocols and/or parameters unless otherwise noted.

“IL-4/IL-13 gene signature,” “IL-4/IL-13 signature,” “IL-13 gene signature,” and “IL-13 signature” are used interchangeably herein and refer to a combination of 30 genes as set forth in Table 4, or a subcombination of these 30 genes as set forth in Table 9, the gene expression pattern of which correlates with certain asthma patients. The 30 genes include POSTN, CST1, CCL26, CLCA1, CST2, PRR4, SERPINB2, CEACAM5, iNOS, SERPINB4, CST4, PRB4, TPSD1, TPSG1, MFSD2, CPA3, GPR105, CDH26, GSN, C20RF32, TRACH2000196 (TMEM71), DNAJC12, RGS13, SLC18A2, SERPINB10, SH3RF2, FCER1B, RUNX2, PTGS1, ALOX15. The polypeptides of the IL-4/IL13 gene signature are “targeted polypeptides” of this invention.

The term “targeted polypeptide” when used herein refers to “native sequence” polypeptides and variants (which are further defined herein).

A “native sequence” polypeptide comprises a polypeptide having the same amino acid sequence as the corresponding polypeptide derived from nature. Thus, the term “native sequence polypeptide” includes naturally-occurring truncated, augmented, and frameshifted forms of a polypeptide, including but not limited to alternatively spliced forms, isoforms and polymorphisms.

“Naturally occurring variant” means a polypeptide having at least about 60% amino acid sequence identity with a reference polypeptide and retains at least one biological activity of the naturally occurring reference polypeptide. Naturally occurring variants can include variant polypeptides having at least about 65% amino acid sequence identity, at least about 70% amino acid sequence identity, at least about 75% amino acid sequence identity, at least about 80% amino acid sequence identity, at least about 80% amino acid sequence identity, at least about 85% amino acid sequence identity, at least about 90% amino acid sequence identity, at least about 95% amino acid sequence identity, at least about 98% amino acid sequence identity or at least about 99% amino acid sequence identity to a reference polypeptide.

Examples of POSTN include a polypeptide comprising SEQ ID NO:1 and other POSTN native sequence polypeptides, such as naturally occurring variants and native sequence polypeptides encoded by a nucleic acid sequence that can hybridize under stringent conditions to SEQ ID NOs:31 and/or 32.

Examples of CST1 include a polypeptide comprising SEQ ID NO:2 and other CST1 native sequence polypeptides, such as naturally occurring variants and native sequence polypeptides encoded by a nucleic acid sequence that can hybridize under stringent conditions to SEQ ID NO:33.

Examples of CCL26 include a polypeptide comprising SEQ ID NO:3 and other CCL26 native sequence polypeptides, such as naturally occurring variants and native sequence polypeptides encoded by a nucleic acid sequence that can hybridize under stringent conditions to SEQ ID NO:34.

Examples of CLCA1 include a polypeptide comprising SEQ ID NO:4 and other CLCA1 native sequence polypeptides, such as naturally occurring variants and native sequence polypeptides encoded by a nucleic acid sequence that can hybridize under stringent conditions to SEQ ID NO:35.

Examples of CST2 include a polypeptide comprising SEQ ID NO:5 and other CST native sequence polypeptides, such as naturally occurring variants and native sequence polypeptides encoded by a nucleic acid sequence that can hybridize under stringent conditions to SEQ ID NO:36.

Examples of PRR4 include a polypeptide comprising SEQ ID NO:6 and other PRR4 native sequence polypeptides, such as naturally occurring variants and native sequence polypeptides encoded by a nucleic acid sequence that can hybridize under stringent conditions to SEQ ID NO:37.

Examples of SERPINB2 include a polypeptide comprising SEQ ID NO:7 and other SERPINB2 native sequence polypeptides, such as naturally occurring variants and native sequence polypeptides encoded by a nucleic acid sequence that can hybridize under stringent conditions to SEQ ID NO:38.

Examples of CEACAM5 include a polypeptide comprising SEQ ID NO:8 and other CEACAM5 native sequence polypeptides, such as naturally occurring variants and native sequence polypeptides encoded by a nucleic acid sequence that can hybridize under stringent conditions to SEQ ID NO:39.

Examples of iNOS include a polypeptide comprising SEQ ID NO:9 and other iNOS native sequence polypeptides, such as naturally occurring variants and native sequence polypeptides encoded by a nucleic acid sequence that can hybridize under stringent conditions to SEQ ID NO:40.

Examples of SERPINB4 include a polypeptide comprising SEQ ID NO:10 and other SERPINB4 native sequence polypeptides, such as naturally occurring variants and native sequence polypeptides encoded by a nucleic acid sequence that can hybridize under stringent conditions to SEQ ID NOs:41 and/or 42.

Examples of CST4 include a polypeptide comprising SEQ ID NO:11 and other CST4 native sequence polypeptides, such as naturally occurring variants and native sequence polypeptides encoded by a nucleic acid sequence that can hybridize under stringent conditions to SEQ ID NO:43.

Examples of PRB4 include a polypeptide comprising SEQ ID NO:12 and other PRB4 native sequence polypeptides, such as naturally occurring variants and native sequence polypeptides encoded by a nucleic acid sequence that can hybridize under stringent conditions to SEQ ID NO:44.

Examples of TPSD1 include a polypeptide comprising SEQ ID NO:13 and other TPSD1 native sequence polypeptides, such as naturally occurring variants and native sequence polypeptides encoded by a nucleic acid sequence that can hybridize under stringent conditions to a sequence selected from the group consisting of SEQ ID NO:45-51.

Examples of TPSG1 include a polypeptide comprising SEQ ID NO:14 and other TPSG1 native sequence polypeptides, such as naturally occurring variants and native sequence polypeptides encoded by a nucleic acid sequence that can hybridize under stringent conditions a sequence selected from the group consisting of SEQ ID NO:52-55.

Examples of MFSD2 include a polypeptide comprising SEQ ID NO:15 and other MFSD2 native sequence polypeptides, such as naturally occurring variants and native sequence polypeptides encoded by a nucleic acid sequence that can hybridize under stringent conditions to SEQ ID NO:56.

Examples of CPA3 include a polypeptide comprising SEQ ID NO:16 and other CPA3 native sequence polypeptides, such as naturally occurring variants and native sequence polypeptides encoded by a nucleic acid sequence that can hybridize under stringent conditions to SEQ ID NO:57.

Examples of GPR105 include a polypeptide comprising SEQ ID NO:17 and other GPR105 native sequence polypeptides, such as naturally occurring variants and native sequence polypeptides encoded by a nucleic acid sequence that can hybridize under stringent conditions to SEQ ID NO:58.

Examples of CDH26 include a polypeptide comprising SEQ ID NO:18 and other CDH26 native sequence polypeptides, such as naturally occurring variants and native sequence polypeptides encoded by a nucleic acid sequence that can hybridize under stringent conditions to SEQ ID NO:59.

Examples of GSN include a polypeptide comprising SEQ ID NO:19 and other GSN native sequence polypeptides, such as naturally occurring variants and native sequence polypeptides encoded by a nucleic acid sequence that can hybridize under stringent conditions to SEQ ID NO:60.

Examples of C20RF32 include a polypeptide comprising SEQ ID NO:20 and other C20RF32 native sequence polypeptides, such as naturally occurring variants and native sequence polypeptides encoded by a nucleic acid sequence that can hybridize under stringent conditions to SEQ ID NO:61.

Examples of TRACH2000196 (TMEM71) include a polypeptide comprising SEQ ID NO:21 and other TRACH2000196 (TMEM71) native sequence polypeptides, such as naturally occurring variants and native sequence polypeptides encoded by a nucleic acid sequence that can hybridize under stringent conditions to SEQ ID NO:62.

Examples of DNAJC12 include a polypeptide comprising SEQ ID NO:22 and other DNAJC12 native sequence polypeptides, such as naturally occurring variants and native sequence polypeptides encoded by a nucleic acid sequence that can hybridize under stringent conditions to SEQ ID NO:63.

Examples of RGS13 include a polypeptide comprising SEQ ID NO:23 and other RGS13 native sequence polypeptides, such as naturally occurring variants and native sequence polypeptides encoded by a nucleic acid sequence that can hybridize under stringent conditions to SEQ ID NO:64.

Examples of SLC18A2 include a polypeptide comprising SEQ ID NO:24 and other SLC18A2 native sequence polypeptides, such as naturally occurring variants and native sequence polypeptides encoded by a nucleic acid sequence that can hybridize under stringent conditions to SEQ ID NO:65.

Examples of SERPINB10 include a polypeptide comprising SEQ ID NO:25 and other SERPINB10 native sequence polypeptides, such as naturally occurring variants and native sequence polypeptides encoded by a nucleic acid sequence that can hybridize under stringent conditions to SEQ ID NO:66.

Examples of SH3RF2 include a polypeptide comprising SEQ ID NO:26 and other SH3RF2 native sequence polypeptides, such as naturally occurring variants and native sequence polypeptides encoded by a nucleic acid sequence that can hybridize under stringent conditions to SEQ ID NO:67.

Examples of FCER1B include a polypeptide comprising SEQ ID NO:27 and other FCER1B native sequence polypeptides, such as naturally occurring variants and native sequence polypeptides encoded by a nucleic acid sequence that can hybridize under stringent conditions to SEQ ID NO:68.

Examples of RUNX2 include a polypeptide comprising SEQ ID NO:28 and other RUNX2 native sequence polypeptides, such as naturally occurring variants and native sequence polypeptides encoded by a nucleic acid sequence that can hybridize under stringent conditions to SEQ ID NO:69.

Examples of PTGS1 include a polypeptide comprising SEQ ID NO:29 and other PTGS1 native sequence polypeptides, such as naturally occurring variants and native sequence polypeptides encoded by a nucleic acid sequence that can hybridize under stringent conditions to SEQ ID NO:70.

Examples of ALOX15 include a polypeptide comprising SEQ ID NO:30 and other ALOX15 native sequence polypeptides, such as naturally occurring variants and native sequence polypeptides encoded by a nucleic acid sequence that can hybridize under stringent conditions to SEQ ID NO:71.

“An anti-IL13/IL4 pathway inhibitor” refers to an agent that blocks the IL-13 and/or IL-4 signalling. Examples of an anti-IL13, anti-IL4 or anti-IL13/IL4 inhibitors include, but are not limited to, anti-IL13 binding agents, anti-IL4 binding agents, anti-IL4receptoralpha binding agents, anti-IL13receptoralpha1 binding agents and anti-IL13 receptoralpha2 binding agents. Single domain antibodies that can bind IL-13, IL-4, IL-13Ralpha1, IL-13Ralpha2 or IL-4Ralpha are specifically included as inhibitors. It should be understood that molecules that can bind more than one target are included.

“Anti-IL4 binding agents” refers to agent that specifically binds to human IL-4. Such binding agents can include a small molecule, an aptamer or a polypeptide. Such polypeptide can include, but is not limited to, a polypeptide(s) selected from the group consisting of an immunoadhesin, an antibody, a peptibody and a peptide. According to one embodiment, the binding agent binds to a human IL-4 sequence with an affinity between 1 uM-1 μM. Specific examples of anti-IL4 binding agents can include soluble IL4Receptor alpha (e.g., extracellular domain of IL4Receptor fused to a human Fc region), anti-IL4 antibody, and soluble IL13receptoralpha1 (e.g., extracellular domain of IL13receptoralpha1 fused to a human Fc region).

“Anti-IL4receptoralpha binding agents” refers to an agent that specifically binds to human IL4 receptoralpha. Such binding agents can include a small molecule, an aptamer or a polypeptide. Such polypeptide can include, but is not limited to, a polypeptide(s) selected from the group consisting of an immunoadhesin, an antibody, a peptibody and a peptide. According to one embodiment, the binding agent binds to a human IL-4 receptor alpha sequence with an affinity between 1 uM-1 μM. Specific examples of anti-IL4 receptoralpha binding agents can include anti-IL4 receptor alpha antibodies.

“Anti-IL13 binding agent” refers to agent that specifically binds to human IL-13. Such binding agents can include a small molecule, aptamer or a polypeptide. Such polypeptide can include, but is not limited to, a polypeptide(s) selected from the group consisting of an immunoadhesin, an antibody, a peptibody and a peptide. According to one embodiment, the binding agent binds to a human IL-13 sequence with an affinity between 1 uM-1 μM. Specific examples of anti-IL13 binding agents can include anti-IL13 antibodies, soluble IL13receptoralpha2 fused to a human Fc, soluble IL4receptoralpha fused to a human Fc, soluble IL13 receptoralpha fused to a human Fc. According to one embodiment, the anti-IL13 antibody comprises the variable domains of the TNX-650 antibody (WO2005/062972). The variable domains of the TNX-650 antibody comprise (1) a VH comprising QVTLRESGPALVKPTQTLTLTCTVSGFSLSAYSVNWIRQPPGKALEWLAMIWGDGKI VYNSALKSRLTISKDTSKNQVVLTMTNMDPVDTATYYCAGDGYYPYAMDNWGQG SLVTVSS (SEQ ID NO:193) and (2) a VL comprising: DIVMTQSPDSLSVSLGERATINCRASKSVDSYGNSFMHWYQQKPGQPPKLLIYLASN LESGVPDRFSGSGSGTDFTLTISSLQAEDVAVYYCQQNNEDPRTFGGGTKVEIK (SEQ ID NO:194). Other examples of anti-IL13 antibodies are described in WO2008/083695 (e.g., IMA-638 and IMA-026), US2008/0267959, US2008/0044420 and US2008/0248048.

Anti-IL13receptoralpha1 binding agents” refers to an agent that specifically binds to human IL13 receptoralpha1. Such binding agents can include a small molecule, aptamer or a polypeptide. Such polypeptide can include, but is not limited to, a polypeptide(s) selected from the group consisting of an immunoadhesin, an antibody, a peptibody and a peptide. According to one embodiment, the binding agent binds to a human IL-13 receptor alpha1 sequence with an affinity between 1 uM-1 μM. Specific examples of anti-IL13 receptoralpha1 binding agents can include anti-IL13 receptor alpha1 antibodies.

“Anti-IL 13receptoralpha2 binding agents” refers to an agent that specifically binds to human IL13 receptoralpha2. Such binding agents can include a small molecule, an aptamer or a polypeptide. Such polypeptide can include, but is not limited to, a polypeptide(s) selected from the group consisting of an immunoadhesin, an antibody, a peptibody and a peptide. According to one embodiment, the binding agent binds to a human IL-13 receptor alpha2 sequence with an affinity between 1 uM-1 μM. Specific examples of anti-IL13 receptoralpha2 binding agents can include anti-IL13 receptor alpha2 antibodies.

“Anti IgE binding agents” refers to an agent that specifically binds to human IgE. Such binding agents can include a small molecule, an aptamer or a polypeptide. Such polypeptide can include, but is not limited to, a polypeptide(s) selected from the group consisting of an immunoadhesin, an antibody, a peptibody and a peptide. According to one embodiment, the anti-IgE antibody comprises a VL sequence comprising Asp Ile Gln Leu Thr Gln Ser Pro Ser Ser Leu Ser Ala Ser Val Gly Asp Arg Val Thr Ile Thr Cys Arg Ala Ser Gln Ser Val Asp Tyr Asp Gly Asp Ser Tyr Met Asn Tip Tyr Gln Gln Lys Pro Gly Lys Ala Pro Lys Leu Leu Ile Tyr Ala Ala Ser Tyr Leu Glu Ser Gly Val Pro Ser Arg Phe Ser Gly Ser Gly Ser Gly Thr Asp Phe Thr Leu Thr Ile Ser Ser Leu Gln Pro Glu Asp Phe Ala Thr Tyr Tyr Cys Gln Gln Ser His Glu Asp Pro Tyr Thr Phe Gly Gln Gly Thr Lys Val Glu Ile Lys Arg Thr Val (SEQ ID NO:213) and a VH sequence comprising Glu Val Gln Leu Val Glu Ser Gly Gly Gly Leu Val Gln Pro Gly Gly Ser Leu Arg Leu Ser Cys Ala Val Ser Gly Tyr Ser Ile Thr Ser Gly Tyr Ser Trp Asn Trp Ile Arg Gln Ala Pro Gly Lys Gly Leu Glu Tip Val Ala Ser Ile Thr Tyr Asp Gly Ser Thr Asn Tyr Asn Pro Ser Val Lys Gly Arg Ile Thr Ile Ser Arg Asp Asp Ser Lys Asn Thr Phe Tyr Leu Gln Met Asn Ser Leu Arg Ala Glu Asp Thr Ala Val Tyr Tyr Cys Ala Arg Gly Ser His Tyr Phe Gly His Trp His Phe Ala Val Tip Gly Gln Gly (SEQ ID NO:214).

“Anti-M1′ binding agents” refers to an agent that specifically binds to the membrane proximal M1′ region of surface expressed IgE on B cells. Such binding agents can include a small molecule, an aptamer or a polypeptide. Such polypeptide can include, but is not limited to, a polypeptide(s) selected from the group consisting of an immunoadhesin, an antibody, a peptibody and a peptide. According to one embodiment, the anti-IgE antibody comprises an antibody described in WO2008/116149 or a variant thereof.

The term “small molecule” refers to an organic molecule having a molecular weight between 50 Daltons to 2500 Daltons.

The term “antibody” is used in the broadest sense and specifically covers, for example, monoclonal antibodies, polyclonal antibodies, antibodies with polyepitopic specificity, single chain antibodies, multi-specific antibodies and fragments of antibodies. Such antibodies can be chimeric, humanized, human and synthetic. Such antibodies and methods of generating them are described in more detail below.

The term “variable” refers to the fact that certain segments of the variable domains differ extensively in sequence among antibodies. The V regions mediate antigen binding and define specificity of a particular antibody for its particular antigen. However, the variability is not evenly distributed across the 110-amino acid span of the variable domains. Instead, the V domains consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” (or “HVR”) when used herein refers to the amino acid residues of an antibody which are responsible for antigen-binding. The hypervariable region generally comprises amino acid residues from a “complementarity determining region” or “CDR” (e.g. around about residues 24-34 (L1), 50-56 (L2) and 89-97 (L3) in the VL, and around about 31-35B (H1), 50-65 (H2) and 95-102 (H3) in the VH (Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the VL, and 26-32 (H1), 52A-55 (H2) and 96-101 (H3) in the VH (Chothia and Lesk J. Mol. Biol. 196:901-917 (1987)).

Hypervariable regions may comprise “extended hypervariable regions” as follows: 24-36 (L1), 46-56 (L2) and 89-97 (L3) in the VL and 26-35B (H1), 47-65 (H2) and 93-102 (H3) in the VH. The variable domain residues are numbered according to Kabat et al., supra for each of these definitions.

“Framework” or “FR” residues are those variable domain residues other than the hypervariable region residues as herein defined. For example, light chain framework 1 (LC-FR1), framework 2 (LC-FR2), framework 3 (LC-FR3) and framework 4 (LC-FR4) region may comprise residues numbered 1-23, 35-49, 57-88 and 98-107 of an antibody (Kabat numbering system), respectively. In another example, heavy chain framework 1 (HC-FR1), heavy chain framework 2 (HC-FR2), heavy chain framework 3 (HC-FR3) and heavy chain framework 4 (HC-FR4) may comprise residues 1-25, 36-48, 66-92 and 103-113, respectively, of an antibody (Kabat numbering system).

As referred to herein, the “consensus sequence” or consensus V domain sequence is an artificial sequence derived from a comparison of the amino acid sequences of known human immunoglobulin variable region sequences.

The term “monoclonal antibody” as used herein refers to an antibody from a population of substantially homogeneous antibodies, i.e., the individual antibodies comprising the population are identical and/or bind the same epitope(s), except for possible variants that may arise during production of the monoclonal antibody, such variants generally being present in minor amounts. Such monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones or recombinant DNA clones. It should be understood that the selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations which typically include different antibodies directed against different determinants (epitopes), each monoclonal antibody of a monoclonal antibody preparations directed against a single determinant on an antigen. In addition to their specificity, the monoclonal antibody preparations are advantageous in that they are typically uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including the hybridoma method (e.g., Kohler et al., Nature, 256:495 (1975); Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681, (Elsevier, N.Y., 1981), recombinant DNA methods (see, e.g., U.S. Pat. No. 4,816,567), phage display technologies (see, e.g., Clackson et al., Nature, 352:624-628 (1991); Marks et al., J. Mol. Biol., 222:581-597 (1991); Sidhu et al., J. Mol. Biol. 338(2):299-310 (2004); Lee et al., J. Mol. Biol 340(5):1073-1093 (2004); Fellouse, Proc. Nat. Acad. Sci. USA 101(34):12467-12472 (2004); and Lee et al. J. Immunol. Methods 284(1-2):119-132 (2004) and technologies for producing human or human-like antibodies from animals that have parts or all of the human immunoglobulin loci or genes encoding human immunoglobulin sequences (see, e.g., WO98/24893, WO/9634096, WO/9633735, and WO/91 10741, Jakobovits et al., Proc. Natl. Acad. Sci. USA, 90:2551 (1993); Jakobovits et al., Nature, 362:255-258 (1993); Bruggemann et al., Year in Immuno., 7:33 (1993); U.S. Pat. Nos. 5,545,806, 5,569,825, 5,591,669 (all of GenPharm); 5,545,807; WO 97/17852, U.S. Pat. Nos. 5,545,807; 5,545,806; 5,569,825; 5,625,126; 5,633,425; and 5,661,016, and Marks et al., Bio/Technology, 10: 779-783 (1992); Lonberg et al., Nature, 368: 856-859 (1994); Morrison, Nature, 368: 812-813 (1994); Fishwild et al., Nature Biotechnology, 14: 845-851 (1996); Neuberger, Nature Biotechnology, 14: 826 (1996); and Lonberg and Huszar, Intern. Rev. Immunol., 13: 65-93 (1995).

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while portions of the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Methods of making chimeric antibodies are known in the art.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. In some embodiments, humanized antibodies are human immunoglobulins (recipient antibody) in which residues from a complementarity-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies may comprise residues which are found neither in the recipient antibody nor in the imported CDR or framework sequences. These modifications are generally made to further refine and maximize antibody performance. Typically, the humanized antibody will comprise substantially all of at least one variable domain, in which all or substantially all of the hypervariable loops derived from a non-human immunoglobulin and all or substantially all of the FR regions are derived from a human immunoglobulin sequence although the FR regions may include one or more amino acid substitutions to, e.g., improve binding affinity. In one preferred embodiment, the humanized antibody will also comprise at least a portion of an immunoglobulin constant region (Fc), typically that of a human immunoglobulin or a human consensus constant sequence. For further details, see Jones et al., Nature, 321:522-525 (1986); Reichmann et al., Nature, 332:323-329 (1988); and Presta, Curr. Op. Struct. Biol., 2:593-596 (1992). The humanized antibody includes a PRIMATIZED® antibody wherein the antigen-binding region of the antibody is derived from an antibody produced by, e.g., immunizing macaque monkeys with the antigen of interest. Methods of making humanized antibodies are known in the art.

Human antibodies can also be produced using various techniques known in the art, including phage-display libraries. Hoogenboom and Winter, J. Mol. Biol., 227:381 (1991); Marks et al., J. Mol. Biol., 222:581 (1991). The techniques of Cole et al. and Boerner et al. are also available for the preparation of human monoclonal antibodies. Cole et al., Monoclonal Antibodies and Cancer Therapy, Alan R. Liss, p. 77 (1985); Boerner et al., J. Immunol., 147(1):86-95 (1991). See also, Lonberg and Huszar, Int. Rev. Immunol. 13:65-93 (1995). PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598.

“Antibody fragments” comprise a portion of a full length antibody, generally the antigen binding or variable region thereof. Examples of antibody fragments include Fab, Fab′, F(ab′)2, and Fv fragments; diabodies; linear antibodies; single-chain antibody molecules; and multispecific antibodies formed from antibody fragments.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) may have the ability to recognize and bind antigen, although at a lower affinity than the entire binding site.

“Functional fragments” of the antibodies of the invention are those fragments that retain binding to polypeptide with substantially the same affinity as the intact full chain molecule from which they are derived and are active in at least one assay (e g, inhibition of TH2-induced asthma pathway such as in mouse models or inhibition of a biological activity of the antigen that binds to the antibody fragment in vitro).

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g. B cell receptor); and B cell activation. A “native sequence Fc region” comprises an amino acid sequence identical to the amino acid sequence of an Fc region found in nature.

“Percent (%) amino acid sequence identity” or “homology” with respect to the polypeptide and antibody sequences identified herein is defined as the percentage of amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc. and the source code has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. The ALIGN-2 program should be compiled for use on a UNIX operating system, preferably digital UNIX V4.0D. All sequence comparison parameters are set by the ALIGN-2 program and do not vary.

The term “Fc region-comprising polypeptide” refers to a polypeptide, such as an antibody or immunoadhesin (see definitions below), which comprises an Fc region. The C-terminal lysine (residue 447 according to the EU numbering system) of the Fc region may be removed, for example, during purification of the polypeptide or by recombinantly engineering the nucleic acid encoding the polypeptide. Accordingly, a composition comprising polypeptides, including antibodies, having an Fc region according to this invention can comprise polypeptides populations with all K447 residues removed, polypeptide populations with no K447 residues removed or polypeptide populations having a mixture of polypeptides with and without the K447 residue.

Throughout the present specification and claims, the Kabat numbering system is generally used when referring to a residue in the variable domain (approximately, residues 1-107 of the light chain and residues 1-113 of the heavy chain) (e.g, Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., Sequences of Proteins of Immunological Interest, 5th Ed. Public Health Service, National Institutes of Health, Bethesda, Md. (1991) expressly incorporated herein by reference). Unless stated otherwise herein, references to residues numbers in the variable domain of antibodies means residue numbering by the Kabat numbering system. Unless stated otherwise herein, references to residue numbers in the constant domain of antibodies means residue numbering by the EU numbering system (e.g., see U.S. Provisional Application No. 60/640,323, Figures for EU numbering).

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, can be identified by those that: (1) employ low ionic strength and high temperature for washing, for example 0.015 M sodium chloride/0.0015 M sodium citrate/0.1% sodium dodecyl sulfate at 50 C; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42 C; or (3) overnight hybridization in a solution that employs 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42 C, with a 10 minute wash at 42 C in 0.2×SSC (sodium chloride/sodium citrate) followed by a 10 minute high-stringency wash consisting of 0.1×SSC containing EDTA at 55 C.

“Moderately stringent conditions” can be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

As used herein, a subject to be treated is a mammal (e.g., human, non-human primate, rat, mouse, cow, horse, pig, sheep, goat, dog, cat, etc.). The subject may be a clinical patient, a clinical trial volunteer, an experimental animal, etc. The subject may be suspected of having or at risk for having asthma or be diagnosed with asthma. According to one preferred embodiment, the subject to be treated according to this invention is a human.

“Treating” or “treatment” or “alleviation” refers to measures, wherein the object is to prevent or slow down (lessen) the targeted pathologic condition or disorder or relieve some of the symptoms of the disorder. Those in need of treatment include can include those already with the disorder as well as those prone to have the disorder or those in whom the disorder is to be prevented. A subject or mammal is successfully “treated” for asthma if, after receiving a therapeutic agent of the present invention, the patient shows observable and/or measurable reduction in or absence of one or more of the following: recurrent wheezing, coughing, trouble breathing, chest tightness, symptoms that occur or worsen at night, symptoms that are triggered by cold air, exercise or exposure to allergens.

The term “therapeutically effective amount” refers to an amount of a polypeptide of this invention effective to “alleviate” or “treat” a disease or disorder in a subject.

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

“Forced expiratory volume (FEV1)” refers to a standard test that measures the volume of air expelled in the first second of a forced expiration. FEV1 is measured by a spirometer, which consists of a mouthpiece and disposable tubing connected to a machine that records the results and displays them on a graph. To perform spirometry, a person inhales deeply, closes the mouth tightly around the tube and then exhales through the tubing while measurements are taken. The volume of air exhaled, and the length of time each breath takes is recorded and analyzed. Spirometry results are expressed as a percentage. Examples of normal spirometry results include a FEV1 of 75 percent of vital capacity after one second. An example of abnormal spirometry results include a reading of less than 80 percent of the normal predicted value. An abnormal result usually indicates the presence of some degree of obstructive lung disease such as asthma, emphysema or chronic bronchitis, or restrictive lung disease such as pulmonary fibrosis. For example, FEV1 values (percentage of predicted) can be used to classify the obstruction that may occur with asthma and other obstructive lung diseases like emphysema or chronic bronchitis: FEV1 65 percent to 79 percent predicted=mild obstruction, FEV1 40 percent to 59 percent predicted=moderate obstruction, and FEV1 less than 40 percent predicted=severe obstruction.

Examples of nucleic acid probes that may be used to identify the proteins described herein (e.g., by microarray analysis), include, but are not limited to the probes described in Table 4.

“Elevated expression level” or “elevated levels” refers to an increased expression of a mRNA or a protein in a patient relative to a control, such as an individual or individuals who are not suffering from asthma.

All publications (including patents and patent applications) cited herein are hereby incorporated in their entirety by reference.

Throughout this specification and claims, the word “comprise,” or variations such as “comprises” or “comprising,” will be understood to imply the inclusion of a stated integer or group of integers but not the exclusion of any other integer or group of integers.

The foregoing written description is considered to be sufficient to enable one skilled in the art to practice the invention. The following Examples are offered for illustrative purposes only, and are not intended to limit the scope of the present invention in any way. Indeed, various modifications of the invention in addition to those shown and described herein will become apparent to those skilled in the art from the foregoing description and fall within the scope of the appended claims.

### PARTIAL LIST OF REFERENCES

1. Haldar, P. and I. D. Pavord, Noneosinophilic asthma: a distinct clinical and pathologic phenotype. J Allergy Clin Immunol, 2007. 119(5): p. 1043-52; quiz 1053-4.
2. Hershey, G. K., IL-13 receptors and signaling pathways: an evolving web. J Allergy Clin Immunol, 2003. 111(4): p. 677-90; quiz 691.
3. Berry, M. A., et al., Sputum and bronchial submucosal IL-13 expression in asthma and eosinophilic bronchitis. J Allergy Clin Immunol, 2004. 114(5): p. 1106-9.
4. Humbert, M., et al., Elevated expression of messenger ribonucleic acid encoding IL-13 in the bronchial mucosa of atopic and nonatopic subjects with asthma. J Allergy Clin Immunol, 1997. 99(5): p. 657-65.
5. Truyen, E., et al., Evaluation of airway inflammation by quantitative Th1/Th2 cytokine mRNA measurement in sputum of asthma patients. Thorax, 2006. 61(3): p. 202-8.
6. Saha, S. K., et al., Increased sputum and bronchial biopsy IL-13 expression in severe asthma. J Allergy Clin Immunol, 2008. 121(3): p. 685-91.
7. Yuyama, N., et al., Analysis of novel disease-related genes in bronchial asthma. Cytokine, 2002. 19(6): p. 287-96.
8. Woodruff, P. G., et al., Genome-wide profiling identifies epithelial cell genes associated with asthma and with treatment response to corticosteroids. Proc Natl Acad Sci U S A, 2007. 104(40): p. 15858-63.
9. Takayama, G., et al., Periostin: a novel component of subepithelial fibrosis of bronchial asthma downstream of IL-4 and IL-13 signals. J Allergy Clin Immunol, 2006. 118(1): p. 98-104.
10. Hayashi, N., et al., T helper 1 cells stimulated with ovalbumin and IL-18 induce airway hyperresponsiveness and lung fibrosis by IFN-gamma and IL-13 production. Proc Natl Acad Sci USA, 2007. 104(37): p. 14765-70.
11. Blanchard, C., et al., IL-13 involvement in eosinophilic esophagitis: transcriptome analysis and reversibility with glucocorticoids. J Allergy Clin Immunol, 2007. 120(6): p. 1292-300.
12. Nakanishi, A., et al., Role of gob-5 in mucus overproduction and airway hyperresponsiveness in asthma. Proc Natl Acad Sci USA, 2001. 98(9): p. 5175-80.
13. Zhou, Y., et al., Characterization of a calcium-activated chloride channel as a shared target of Th2 cytokine pathways and its potential involvement in asthma. Am J Respir Cell Mol Biol, 2001. 25(4): p. 486-91.
14. Kuperman, D. A., et al., Dissecting asthma using focused transgenic modeling and functional genomics. J Allergy Clin Immunol, 2005. 116(2): p. 305-11.
15. Ray, R., et al., Uteroglobin suppresses SCCA gene expression associated with allergic asthma. J Biol Chem, 2005. 280(11): p. 9761-4.
16. Sabatini, L. M., et al., Tissue distribution of RNAs for cystatins, histatins, statherin, and proline-rich salivary proteins in humans and macaques. J Dent Res, 1989. 68(7): p. 1138-45.
17. Lindahl, M., B. Stahlbom, and C. Tagesson, Newly identified proteins in human nasal and bronchoalveolar lavage fluids: potential biomedical and clinical applications. Electrophoresis, 1999. 20(18): p. 3670-6.
18. Cimerman, N., et al., Serum cystatin C, a potent inhibitor of cysteine proteinases, is elevated in asthmatic patients. Clin Chim Acta, 2000. 300(1-2): p. 83-95.
19. Finkelman, F. D., et al., Suppressive effect of IL-4 on IL-13-induced genes in mouse lung. J Immunol, 2005. 174(8): p. 4630-8.
20. Zimmermann, N., et al., Expression and regulation of small proline-rich protein 2 in allergic inflammation. Am J Respir Cell Mol Biol, 2005. 32(5): p. 428-35.
21. Warner, T. F. and E. A. Azen, Proline-rich proteins are present in serous cells of submucosal glands in the respiratory tract. Am Rev Respir Dis, 1984. 130(1): p. 115-8.
22. Maeda, Y., et al., [Concentrations of carcinoembryonic antigen in serum and bronchoalveolar lavage fluid of asthmatic patients with mucoid impaction]. Nihon Kokyuki Gakkai Zasshi, 2004. 42(12): p. 988-93.
23. Suresh, V., J. D. Mih, and S. C. George, Measurement of IL-13-induced iNOS-derived gas phase nitric oxide in human bronchial epithelial cells. Am J Respir Cell Mol Biol, 2007. 37(1): p. 97-104.
24. Storey, J. D. and R. Tibshirani, Statistical significance for genomewide studies. Proc Natl Acad Sci USA, 2003. 100(16): p. 9440-5.
25. Eisen, M. B., et al., Cluster analysis and display of genome-wide expression patterns. Proc Natl Acad Sci USA, 1998. 95(25): p. 14863-8.
26. Saldanha, A. J., Java Treeview—extensible visualization of microarray data. Bioinformatics, 2004. 20(17): p. 3246-8.
27. Kelly-Welch, A. E., Hanson, E. M., Boothby, M. R. & Keegan, A. D. Interleukin-4 and interleukin-13 signaling connections maps. Science 300, 1527-8 (2003).
28. Fixman, E. D., Stewart, A. & Martin, J. G. Basic mechanisms of development of airway structural changes in asthma. Eur Respir J 29, 379-89 (2007).
29. Holgate, S. T. Pathogenesis of asthma. Clin Exp Allergy 38, 872-97 (2008).
30. Roche, W. R., Beasley, R., Williams, J. H. & Holgate, S. T. Subepithelial fibrosis in the bronchi of asthmatics. Lancet 1, 520-4 (1989).
31. Ferrando, R. E., Nyengaard, J. R., Hays, S. R., Fahy, J. V. & Woodruff, P. G. Applying stereology to measure thickness of the basement membrane zone in bronchial biopsy specimens. J Allergy Clin Immunol 112, 1243-5 (2003).
32. Ordonez, C. L. et al. Mild and moderate asthma is associated with airway goblet cell hyperplasia and abnormalities in mucin gene expression. Am J Respir Crit Care Med 163, 517-23 (2001).
33. Martin, R. J. et al. The Predicting Response to Inhaled Corticosteroid Efficacy (PRICE) trial. J Allergy Clin Immunol 119, 73-80 (2007).
34. Wenzel, S., Wilbraham, D., Fuller, R., Getz, E. B. & Longphre, M. Effect of an interleukin-4 variant on late phase asthmatic response to allergen challenge in asthmatic patients: results of two phase 2a studies. Lancet 370, 1422-31 (2007).
35. Grunig, G. et al. Requirement for IL-13 independently of IL-4 in experimental asthma. Science 282, 2261-3 (1998).
36. Wills-Karp, M. et al. Interleukin-13: central mediator of allergic asthma. Science 282, 2258-61 (1998).
37. Simpson, J. L., Scott, R., Boyle, M. J. & Gibson, P. G. Inflammatory subtypes in asthma: assessment and identification using induced sputum. Respirology 11, 54-61 (2006).
38. Wenzel, S. E. et al. Evidence that severe asthma can be divided pathologically into two inflammatory subtypes with distinct physiologic and clinical characteristics. Am J Respir Crit Care Med 160, 1001-8 (1999).
39. Wenzel, S. E. Asthma: defining of the persistent adult phenotypes. Lancet 368, 804-13 (2006).
40. Berry, M. et al. Pathological features and inhaled corticosteroid response of eosinophilic and non-eosinophilic asthma. Thorax 62, 1043-9 (2007).
41. Pavord, I. D., Brightling, C. E., Woltmann, G. & Wardlaw, A. J. Non-eosinophilic corticosteroid unresponsive asthma. Lancet 353, 2213-4 (1999).
42. McKinley, L. et al. TH17 cells mediate steroid-resistant airway inflammation and airway hyperresponsiveness in mice. J Immunol 181, 4089-97 (2008).
43. Holgate, S. T. Epithelium dysfunction in asthma. J Allergy Clin Immunol 120, 1233-44; quiz 1245-6 (2007).
44. Martin, R. J., Kraft, M., Chu, H. W., Berns, E. A. & Cassell, G. H. A link between chronic asthma and chronic infection. J Allergy Clin Immunol 107, 595-601 (2001).
45. Dolganov, G. M. et al. A novel method of gene transcript profiling in airway biopsy homogenates reveals increased expression of a Na+-K+-C1-cotransporter (NKCC1) in asthmatic subjects. Genome Res 11, 1473-83 (2001).
46. Innes, A. L. et al. Epithelial mucin stores are increased in the large airways of smokers with airflow obstruction. Chest 130, 1102-8 (2006).
47. Gentleman, R. C. et al. Bioconductor: open software development for computational biology and bioinformatics. Genome Biol 5, R80 (2004).
48. Douwes J, Gibson P, Pekkanen J, Pearce N. Non-eosinophilic asthma: importance and possible mechanisms. Thorax 2002; 57(7):643-8.
49. Galli S J, Tsai M, Piliponsky A M. The development of allergic inflammation. Nature 2008; 454(7203):445-54.
50. Barnes P J. The cytokine network in asthma and chronic obstructive pulmonary disease. J Clin Invest 2008; 118(11):3546-56.
51. Woodruff P G, Koth L L, Yang Y H, et al. A distinctive alveolar macrophage activation state induced by cigarette smoking Am J Respir Crit Care Med 2005; 172(11):1383-92.
52. Weibel E R, Hsia C C, Ochs M. How much is there really? Why stereology is essential in lung morphometry. J Appl Physiol 2007; 102(1):459-67.
53. Seibold M A, Donnelly S, Solon M, et al. Chitotriosidase is the primary active chitinase in the human lung and is modulated by genotype and smoking habit. J Allergy Clin Immunol 2008; 122(5):944-50 e3.
54. Kim E Y, Battaile J T, Patel A C, et al. Persistent activation of an innate immune response translates respiratory viral infection into chronic lung disease. Nat Med 2008; 14(6):633-40.
55. Peters-Golden M. The alveolar macrophage: the forgotten cell in asthma. Am J Respir Cell Mol Biol 2004; 31(1):3-7.
56. Chu H W, Balzar S, Westcott J Y, et al. Expression and activation of 15-lipoxygenase pathway in severe asthma: relationship to eosinophilic phenotype and collagen deposition. Clin Exp Allergy 2002; 32(11):1558-65.
57. Gordon S. Alternative activation of macrophages. Nat Rev Immunol 12003; 3(1):23-35.
58. Anderson G P. Endotyping asthma: new insights into key pathogenic mechanisms in a complex, heterogeneous disease. Lancet 2008; 372(9643):1107-19.
59. Berry M A, Hargadon B, Shelley M, et al. Evidence of a role of tumor necrosis factor alpha in refractory asthma. N Engl J Med 2006; 354(7):697-708.
60. Takatsu K, Nakajima H. IL-5 and eosinophilia. Curr Opin Immunol 2008; 20(3):288-94.
61. Zimmermann N, Hershey G K, Foster P S, Rothenberg M E. Chemokines in asthma: cooperative interaction between chemokines and IL-13. J Allergy Clin Immunol 2003; 111(2):227-42; quiz 43.
62. Flood-Page P T, Menzies-Gow A N, Kay A B, Robinson D S. Eosinophil's role remains uncertain as anti-interleukin-5 only partially depletes numbers in asthmatic airway. Am J Respir Crit Care Med 2003; 167(2):199-204.
63. Flood-Page P, Swenson C, Faiferman I, et al. A study to evaluate safety and efficacy of mepolizumab in patients with moderate persistent asthma. Am J Respir Crit Care Med 2007; 176(11):1062-71.
64. Baril, P., et al., Periostin promotes invasiveness and resistance of pancreatic cancer cells to hypoxia-induced cell death: role of the beta4 integrin and the PI3k pathway. Oncogene, 2007: 26(14): p. 2082-94.
65. Kudo, Y., et al., Periostin promotes invasion and anchorage-independent growth in the metastatic process of head and neck cancer. Cancer Res, 2006:66(14): p. 6928-35
66. Puglisi, F., et al., Expression of periostin in human breast cancer. J Clin Pathol, 2008:61(4): p. 494-8. 15.
67. Siriwardena, B. S., et al., Periostin is frequently overexpressed and enhances invasion and angiogenesis in oral cancer. Br J Cancer, 2006:95(10): p. 1396-403.
68. Sasaki, H., et al., Serum level of the periostin, a homologue of an insect cell adhesion molecule, as a prognostic marker in nonsmall cell lung carcinomas. Cancer, 2001:92(4): p. 843-8.
69. Sasaki, H., et al., Expression of Periostin, homologous with an insect cell adhesion molecule, as a prognostic marker in non-small cell lung cancers. Jpn J Cancer Res, 2001:92(8): p. 869-73.
70. Sasaki, H., et al., Elevated serum periostin levels in patients with bone metastases from breast but not lung cancer. Breast Cancer Res Treat, 2003:77(3): p. 245-52.
71. Komiya, A., et al., Concerted expression of eotaxin-1, eotaxin-2, and eotaxin-3 in human bronchial epithelial cells. Cell Immunol, 2003:225(2): p. 91-100.
72. Chupp, G. L., et al., A chitinase-like protein in the lung and circulation of patients with severe asthma. N Engl J Med, 2007:357(20): p. 2016-27.
73. Liu, S. M., et al., Immune cell transcriptome datasets reveal novel leukocyte subset-specific genes and genes associated with allergic processes. J Allergy Clin Immunol, 2006:118(2): p. 496-503.
74. Nakajima, T., et al., Identification of granulocyte subtype-selective receptors and ion channels by using a high-density oligonucleotide probe array. J Allergy Clin Immunol, 2004:113(3): p. 528-35.
75. De Filippis, D., A. D'Amico, and T. Iuvone, Cannabinomimetic control of mast cell mediator release: new perspective in chronic inflammation. J Neuroendocrinol, 2008:20 Suppl 1: p. 20-5.
76. Reed, C. E. and H. Kita, The role of protease activation of inflammation in allergic respiratory diseases. J Allergy Clin Immunol, 2004:114(5): p. 997-1008; quiz 1009.
77. Blanchard, C., et al., Periostin facilitates eosinophil tissue infiltration in allergic lung and esophageal responses. Mucosal Immunol, 2008:1(4): p. 289-96.
78. Wenzel, S. E., et al., Bronchoscopic evaluation of severe asthma. Persistent inflammation associated with high dose glucocorticoids. Am J Respir Crit Care Med, 1997:156(3 Pt 1): p. 737-43.
79. Leung, D. Y., et al., Dysregulation of interleukin 4, interleukin 5, and interferon gamma gene expression in steroid-resistant asthma. J Exp Med, 1995:181(1): p. 33-40.
80. Walsh, G. M., Emerging drugs for asthma. Expert Opin Emerg Drugs, 2008:13(4): p. 643-53.
81. Ballesta, A. M., et al., Carcinoembryonic antigen in staging and follow-up of patients with solid tumors. Tumour Biol, 1995:16(1): p. 32-41.

All references cited herein, including patent applications and publications, are incorporated by reference in their entirety for any purpose. In addition, U.S. Provisional Applications U.S. Ser. No. 61/072,572, filed Mar. 31, 2008, U.S. Ser. No. 61/041,480, filed Apr. 1, 2008, U.S. Ser. No. 61/128,383, filed May 20, 2008, U.S. Ser. No. 61/205,392, filed Jan. 16, 2009 are incorporated by reference in their entirety. Also, specifically PCT publications WO2005/062972 and WO2008/116149 are incorporated by reference by their entirety.

### EXAMPLES

### Example 1

### Methods

### Airway Tissue Bank

We studied biological samples stored in the Airway Tissue Bank at the University of California, San Francisco (UCSF) that had been collected during bronchoscopy performed for research purposes in healthy and asthmatic volunteers. Research bronchoscopy had included collection of epithelial brushings, bronchoalveolar lavage (BAL) and bronchial biopsies using specific methods previously described [8, 46]. BAL cell counts and differentials had been performed and databased, and macrophages had been sorted from BAL fluid using flow cytometry [51]. Four to six bronchial biopsies had been obtained from 2nd-through 5th-order carinae (contralateral to the brushing site), formalin-fixed, and then paraffin-embedded in isotropic uniform random orientation [31] to enable quantitative measures of inflammation and remodeling using methods of design-based stereology [52]. An additional 2 bronchial biopsies had been homogenized and processed for RNA using the Qiagen RNeasy minikit (Qiagen Inc., Valencia, Calif.). RNA extracted from epithelial brushings, homogenates of bronchial biopsies, and lavage macrophages had been quality assured and aliquoted for future microrray- and PCR-based gene profiling. All research bronchoscopy studies had been approved by the UCSF Committee on Human Research (CHR), written informed consent had been obtained from all subjects, and all studies had been performed in accordance with the principles expressed in the Declaration of Helsinki The Airway Tissue Bank procedures were also reviewed and approved by UCSF's CHR. Samples of epithelial brushings and macrophages from this tissue bank have been used in previously reported studies [8, 14, 46, 51, 53]. Most recently, microarray analyses of differentially expressed genes in epithelial brushings in asthmatic subjects have been reported by us [8].

For the purposes of identifying subsets of patients with asthma who differ with respect to the molecular mechanism underlying their airway inflammation and the distinct inflammatory, pathological and clinical phenotypes characteristic of these subsets, we first conducted new analyses on our previously generated epithelial cell microarray data, and we then supplemented these new analyses with review of additional and detailed clinical characterization data (including data on bronchodilator reversibility and allergen skin test reactivity) from these same subjects and newly generated data, including: (i) gene expression profiles in homogenates of bronchial biopsies and alveolar macrophages; (ii) quantitative measures of subepithelial collagen and airway epithelial mucin in bronchial biopsies; (iii) total and differential cell counts in BAL.

### Human Subjects and Samples

Subjects with asthma (N=42) had a prior physician diagnosis of asthma, symptoms consistent with asthma confirmed by a study physician, airway hyper-responsiveness (defined as a drop in forced expiratory volume in the first second (FEV₁) of 20% or greater with inhalation of <8 mg/mL of methacholine [PC₂₀ methacholine] and either: 1) symptoms on 2 or more days per week, 2) β-agonist use on 2 or more days per week, or 3) an FEV₁<85% predicted. They did not take inhaled or oral corticosteroids for 4 weeks prior to enrollment. Healthy controls (N=27) had no history of lung disease and lacked airway hyper-responsiveness (PC₂₀ methacholine >16 mg/mL). Certain studies included current smokers without asthma (N=16). Exclusion criteria for all subjects included upper respiratory tract infection in the previous 4 weeks, asthma exacerbation within 6 weeks and current use of salmeterol, astemizole, nedocromil sodium, sodium cromoglycate, methlyxanthines, montelukast or zafirlukast. Subjects underwent baseline evaluation by study physicians (including spirometry and methacholine challenge testing as described previously [8]). Subjects also underwent allergen skin prick testing (ASPT) with a panel of 12 aeroallergens, a positive control and a negative control (Table 6).

Thirty-two of the subjects with asthma had also been enrolled in a double-blind randomized controlled clinical trial of inhaled fluticasone (500 μg, twice daily, N=19) or matched placebo (N=13) (ClinicalTrials.gov Identifier: NCT00187499). The trial was designed to determine the effects of inhaled steroid (fluticasone) on airway gene expression and to relate gene expression changes to improvements in lung function. The asthma subjects in the clinical trial had undergone baseline bronchoscopy and had been randomized to receive study medication before undergoing repeat bronchoscopy one week later after starting study drug. Asthma subjects continued study medication for a total of 8 weeks. Healthy control subjects and smokers were enrolled in one of three cross-sectional studies, which comprised two visits each, the first for characterization and the second for bronchoscopy 1 week later. Thirty-five subjects had adequate baseline bronchoscopy, and 32 had RNA available from epithelial brushings at both bronchoscopies. Lung function was measured (by spirometry) after 4 weeks and 8 weeks on study medication, and a final spirometry was completed after a one week run-out. Methods for bronchoscopy, epithelial brushing, bronchoalveaolar lavage, spirometry, and sample handling were identical across all studies.

Bronchoalveolar lavage (BAL) was performed by instilling 4 aliquots of 50 ml of sterile saline into either the lingula or right middle lobe, with recovery by suction. Cell counts were performed using a hemocytometer and Turks solution (1% glacial acetic acid and 0.01% gentian violet in distilled H₂O). Then BAL cell differentials were performed on cytocentrifuged preparations using the Shandon Kwik-Diff stain kit (Thermo Fisher Scientific, Waltham Mass.). Thirty-two of the subjects with asthma were also enrolled in a double-blind randomized controlled clinical trial of inhaled fluticasone (500 mcg BID) or matched placebo. In addition to the inclusion criteria above, these subjects were also required to have either asthma symptoms on 2 or more days per week, or β-agonist use on 2 or more days per week, or FEV₁<85% predicted. Subjects in the clinical trial underwent a baseline visit and baseline bronchoscopy as described above, were randomized to receive study medication and underwent repeat bronchoscopy one week later. Then, they continued study medication for a total of 8 weeks with scheduled re-assessment of spirometry and methacholine challenge testing. All clinical studies were approved by the University of California at San Francisco Committee on Human Research, written informed consent was obtained from all subjects, and all studies were performed in accordance with the principles expressed in the Declaration of Helsinki

### Microarray Analyses and Morphometry

Microarray data from mild-moderate non-smoking asthma patients and healthy non-smoking subjects were obtained from a previous study as described [8]. Methodological detail and microarray data are also available from the Gene Expression Omnibus public database, which can be accessed online at the National Center for Biotechnology Information, accession number GSE4302. Microarray data was analysed in the present study to determine whether genes were differentially regulated within the asthmatic group. Also, the microarray data was analyzed to determine whether other genes were co-regulated with top asthma-related, IL-13 induced genes. Two step real-time PCR (qPCR) was performed as described previously [45] using the primers and probes in Table 1 (i.e., multiplex PCR followed by real time PCR on cDNA generated products).

Morphometric analyses were performed by applying design-based stereology to 4-6 endobronchial biopsies from each subject as described previously. Specifically, analysis of reticular basement membrane thickness was measured in trichrome 3 μm sections using the orthogonal intercept method [31]. Airway mucin content was measured in Alcian blue/Periodic acid Schiff 3 μm sections using point and line intersect counting methods [46].

### Statistical Methods

Microarray preprocessing was performed using RMA with Bioconductor open source software [47] in the R statistical environment. Unsupervised hierarchical clustering was performed using the Euclidean metric with complete linkage. All other statistical analyses including were performed using the JMP statistical analysis software package (SAS Institute, Cary, N.C.). Values are presented as mean±standard deviation or median (range) unless otherwise specified. Correlation was performed using Spearman's rank order correlation. For significance testing of PC₂₀ and serum IgE levels, data were log transformed for normality. A p<0.05 was taken as statistically significant and sidak correction for multiple comparisons was employed after initial three-group comparisons by ANOVA.

TABLE 1
Primer and probe sequences for qPCR

| Gene | Type | Sequence |
|---|---|---|
| IL-13 | RT-forward | GGATGCTGAGCGGATTCTG |
| | | [SEQ ID NO: 73] |
| | RT-reverse | CCCTCGCGAAAAAGTTTCTT |
| | | [SEQ ID NO: 74] |
| | Taqman-forward | AAGGTCTCAGCTGGGCAGTTT |
| | | [SEQ ID NO: 75] |
| | Taqman-reverse | AAACTGGGCCACCTCGATT |
| | | [SEQ ID NO: 76] |
| | probe | CCAGCTTGCATGTCCGAGACACCA |
| | | [SEQ ID NO: 77] |
| IL-4 | RT-forward | GGGTCTCACCTCCCAACTGC |
| | | [SEQ ID NO: 78] |
| | RT-reverse | TGTCTGTTACGGTCAACTCGGT |
| | | [SEQ ID NO: 79] |
| | Taqman-forward | GCTTCCCCCTCTGTTCTTCCT |
| | | [SEQ ID NO: 80] |
| | Taqman-reverse | GCTCTGTGAGGCTGTTCAAAGTT |
| | | [SEQ ID NO: 81] |
| | probe | TCCACGGACACAAGTGCGATATCACC |
| | | [SEQ ID NO: 82] |
| IL-5 | RT-forward | GCCATGAGGATGCTTCTGCA |
| | | [SEQ ID NO: 83] |
| | RT-reverse | GAATCCTCAGAGTCTCATTGGCTATC |
| | | [SEQ ID NO: 84] |
| | Taqman-forward | AGCTGCCTACGTGTATGCCA |
| | | [SEQ ID NO: 85] |
| | Taqman-reverse | GTGCCAAGGTCTCTTTCACCA |
| | | [SEQ ID NO: 86] |
| | probe | CCCCACAGAAATTCCCACAAGTGCA |
| | | [SEQ ID NO: 87] |
| MUC2 | RT-forward | ACTCCTCTACCTCCATCAATAACTCC |
| | | [SEQ ID NO: 88] |
| | RT-reverse | TGGCTCTGCAAGAGATGTTAGCT |
| | | [SEQ ID NO: 89] |
| | Taqman-forward | GCTGGCTGGATTCTGGAAAA |
| | | [SEQ ID NO: 90] |
| | Taqman-reverse | TGGCTCTGCAAGAGATGTTAGC |
| | | [SEQ ID NO: 91] |
| | probe | TCTCCAATCAATTCTGTGTCTCCACCTG |
| | | G |
| | | [SEQ ID NO: 92] |
| MUC5ac2 | RT-forward | TGTGGCGGGAAAGACAGC |
| | | [SEQ ID NO: 93] |
| | RT-reverse | CCTTCCTATGGCTTAGCTTCAGC |
| | | [SEQ ID NO: 94] |
| | Taqman-forward | CGTGTTGTCACCGAGAACGT |
| | | [SEQ ID NO: 95] |
| | Taqman-reverse | ATCTTGATGGCCTTGGAGCA |
| | | [SEQ ID NO: 96] |
| | probe | CTGCGGCACCACAGGGACCA |
| | | [SEQ ID NO: 97] |
| MUC5b | RT-forward | TTGAGGACCCCTGCTCCCT |
| | | [SEQ ID NO: 98] |
| | RT-reverse | AGGCGTGCACATAGGAGGAC |
| | | [SEQ ID NO: 99] |
| | Taqman-forward | CGATCCCAACAGTGCCTTCT |
| | | [SEQ ID NO: 100] |
| | Taqman-reverse | CCTCGCTCCGCTCACAGT |
| | | [SEQ ID NO: 101] |
| | probe | CAACCCCAAGCCCTTCCACTCGA |
| | | [SEQ ID NO: 102] |
| ALOX15 | RT-forward | CCAACCACCAAGGATGCAA |
| | | [SEQ ID NO: 103] |
| | RT-reverse | TCTGCCCAGCTGCCAAGT |
| | | [SEQ ID NO: 104] |
| | Taqman-forward | CCAACCACCAAGGATGCAA |
| | | [SEQ ID NO: 105] |
| | Taqman-reverse | GGAGAGAAGCCTGGTGGAAGT |
| | | [SEQ ID NO: 106] |
| | probe | CAGTGTCGCCATCACTGTCTCCAGC |
| | | [SEQ ID NO: 107] |
| ALOX5 | RT-forward | ACGTCCACCAGACCATCACC |
| | | [SEQ ID NO: 108] |
| | RT-reverse | GAATCTCACGTGTGCCACCA |
| | | [SEQ ID NO: 109] |
| | Taqman-forward | ATTGCAATGTACCGCCAGC |
| | | [SEQ ID NO: 110] |
| | Taqman-reverse | GAATCTCACGTGTGCCACCA |
| | | [SEQ ID NO: 111] |
| | probe | CTGCTGTGCACCCCATTTTCAAGCTG |
| | | [SEQ ID NO: 112] |
| ALOX5AP | RT-forward | CATAAAGTGGAGCACGAAAGCA |
| | | [SEQ ID NO: 113] |
| | RT-reverse | GGTACGCATCTACACAGTTCTGGTT |
| | | [SEQ ID NO: 114] |
| | Taqman-forward | CAGAATGGGAGGAGCTTCCA |
| | | [SEQ ID NO: 115] |
| | Taqman-reverse | CACAGTTCTGGTTGGCAGTGTAG |
| | | [SEQ ID NO: 116] |
| | probe | CCGGAACACTTGCCTTTGAGCGG |
| | | [SEQ ID NO: 117] |
| ARG1 | RT-forward | CAAGGTCTGTGGGAAAAGCAA |
| | | [SEQ ID NO: 118] |
| | RT-reverse | TGGCCAGAGATGCTTCCAAT |
| | | [SEQ ID NO: 119] |
| | Taqman-forward | GCAGAAGTCAAGAAGAACGGAAGA |
| | | [SEQ ID NO: 120] |
| | Taqman-reverse | TGCTTCCAATTGCCAAACTG |
| | | [SEQ ID NO: 121] |
| | probe | TCTCCGCCCAGCACCAGGCT |
| | | [SEQ ID NO: 122] |
| IL1B | RT-forward | ACTTAAAGCCCGCCTGACAGA |
| | | [SEQ ID NO: 123] |
| | RT-reverse | GCTACTTCTTGCCCCCTTTGAA |
| | | [SEQ ID NO: 124] |
| | Taqman-forward | CCACGGCCACATTTGGTT |
| | | [SEQ ID NO: 125] |
| | Taqman-reverse | AGGGAAGCGGTTGCTCATC |
| | | [SEQ ID NO: 126] |
| | probe | AGAAACCCTCTGTCATTCGCTCCCACAT |
| | | [SEQ ID NO: 127] |
| IL 1rn | RT-forward | CTCCGCAGTCACCTAATCACTCT |
| | | [SEQ ID NO: 128] |
| | RT-reverse | GGCTCAATGGGTACCACATCTATCT |
| | | [SEQ ID NO: 129] |
| | Taqman-forward | TTCCTGTTCCATTCAGAGACGAT |
| | | [SEQ ID NO: 130] |
| | Taqman-reverse | AGATTCTGAAGGCTTGCATCTTG |
| | | [SEQ ID NO: 131] |
| | probe | TGCCGACCCTCTGGGAGAAAATCC |
| | | [SEQ ID NO: 132] |
| LTA4H | RT-forward | ATTCAAGGATCTTGCTGCCTTT |
| | | [SEQ ID NO: 133] |
| | RT-reverse | TGCAGTCACGGGATGCAT |
| | | [SEQ ID NO: 134] |
| | Taqman-forward | CAAGGATCTTGCTGCCTTTGA |
| | | [SEQ ID NO: 135] |
| | Taqman-reverse | TGCTTGCTTTGTGCTCTTGGT |
| | | [SEQ ID NO: 136] |
| | probe | AAATCCCATGATCAAGCTGTCCGAACC |
| | | [SEQ ID NO: 137] |
| LTC4S | RT-forward | CACCACACCGACGGTACCA |
| | | [SEQ ID NO: 138] |
| | RT-reverse | TGCGCGCCGAGATCA |
| | | [SEQ ID NO: 139] |
| | Taqman-forward | CCATGAAGGACGAGGTAGCTCTA |
| | | [SEQ ID NO: 140] |
| | Taqman-reverse | TGCGCGCCGAGATCA |
| | | [SEQ ID NO: 141] |
| | probe | CCTGGGAGTCCTGCTGCAAGCCTACT |
| | | [SEQ ID NO: 142] |
| MRC1 | RT-forward | CGCTACTAGGCAATGCCAATG |
| | | [SEQ ID NO: 143] |
| | RT-reverse | GCAATCTGCGTACCACTTGTTTT |
| | | [SEQ ID NO: 144] |
| | Taqman-forward | CGCTACTAGGCAATGCCAATG |
| | | [SEQ ID NO: 145] |
| | Taqman-reverse | GCAATCTGCGTACCACTTGTTTT |
| | | [SEQ ID NO: 146] |
| | probe | AGCAACCTGTGCATTCCCGTTCAAGT |
| | | [SEQ ID NO: 147] |
| MRC2 | RT-forward | GGGAGCACTGCTATTCTTTCCA |
| | | [SEQ ID NO: 148] |
| | RT-reverse | CAAACACATTCTCCATCTCATCCA |
| | | [SEQ ID NO: 149] |
| | Taqman-forward | GAGCACTGCTATTCTTTCCACATG |
| | | [SEQ ID NO: 150] |
| | Taqman-reverse | TCTCCATCTCATCCAGGATAGACA |
| | | [SEQ ID NO: 151] |
| | probe | CCACCCGCTCTCTGGCAGCG |
| | | [SEQ ID NO: 152] |
| SCYA22 | RT-forward | GCATGGCTCGCCTACAGACT |
| | | [SEQ ID NO: 153] |
| | RT-reverse | CAGACGGTAACGGACGTAATCAC |
| | | [SEQ ID NO: 154] |
| | Taqman-forward | TGGCGCTTCAAGCAACTG |
| | | [SEQ ID NO: 155] |
| | Taqman-reverse | CAGACGGTAACGGACGTAATCA |
| | | [SEQ ID NO: 156] |
| | probe | AGGCCCCTACGGCGCCAACAT |
| | | [SEQ ID NO: 157] |
| TNFa | RT-forward | CTGGTATGAGCCCATCTATCTGG |
| | | [SEQ ID NO: 158] |
| | RT-reverse | TTGGATGTTCGTCCTCCTCAC |
| | | [SEQ ID NO: 159] |
| | Taqman-forward | GGAGAAGGGTGACCGACTCA |
| | | [SEQ ID NO: 160] |
| | Taqman-reverse | TGCCCAGACTCGGCAAAG |
| | | [SEQ ID NO: 161] |
| | probe | CGCTGAGATCAATCGGCCCGACTA |
| | | [SEQ ID NO: 162] |
| SCYA20 | RT-forward | GGCTGTGACATCAATGCTATCATC |
| | | [SEQ ID NO: 163] |
| | RT-reverse | GTCCAGTGAGGCACAAATTAGATAAG |
| | | [SEQ ID NO: 164] |
| | Taqman-forward | TCTGGAATGGAATTGGACATAGCCCAAG |
| | | [SEQ ID NO: 165] |
| | Taqman-reverse | CCAACCCCAGCAAGGTTCTTTCTG |
| | | [SEQ ID NO: 166] |
| | probe | ACCCTCCATGATGTGCAAGTGAAACC |
| | | [SEQ ID NO: 167] |
| SCYA17 | RT-forward | GGATGCCATCGTTTTTGTAACTG |
| | | [SEQ ID NO: 168] |
| | RT-reverse | CCTCTCAAGGCTTTGCAGGTA |
| | | [SEQ ID NO: 169] |
| | Taqman-forward | GGGCAGGGCCATCTGTTC |
| | | [SEQ ID NO: 170] |
| | Taqman-reverse | TCTCAAGGCTTTGCAGGTATTTAA |
| | | [SEQ ID NO: 171] |
| | probe | ACCCCAACAACAAGAGAGTGAAGAATGC |
| | | A |
| | | [SEQ ID NO: 172] |
| IL12A | RT-forward | CCTCCTCCTTGTGGCTACCC |
| | | [SEQ ID:173] |
| | RT-reverse | CAATCTCTTCAGAAGTGCAAGGG |
| | | [SEQ ID: 174] |
| | Taqman-forward | TCCTCCTGGACCACCTCAGT |
| | | [SEQ ID: 175] |
| | Taqman-reverse | GAACATTCCTGGGTCTGGAGTG |
| | | [SEQ ID: 176] |
| | Probe | TGGCCAGAAACCTCCCCGTGG |
| | | [SEQ ID: 177] |
| IFNγ | RT-forward | GTAACTGACTTGAATGTCCAACGC |
| | | [SEQ ID: 178] |
| | RT-reverse | GACAACCATTACTGGGATGCTC |
| | | [SEQ ID: 179] |
| | Taqman-forward | CCAACGCAAAGCAATACATGA |
| | | [SEQ ID: 180] |
| | Taqman-reverse | TTTTCGCTTCCCTGTTTTAGCT |
| | | [SEQ ID: 181] |
| | Probe | TCCAAGTGATGGCTGAACTGTCGCC |
| | | [SEQ ID: 182] |
| IL-10 | RT-forward | GTTGCCTGGTCCTCCTGACT |
| | | [SEQ ID: 183] |
| | RT-reverse | TGTCCAGCTGATCCTTCATTTG |
| | | [SEQ ID: 184] |
| | Taqman-forward | TGAGAACAGCTGCACCCACTT |
| | | [SEQ ID: 185] |
| | Taqman-reverse | GCTGAAGGCATCTCGGAGAT |
| | | [SEQ ID: 186] |
| | Probe | CAGGCAACCTGCCTAACATGCTTCG |
| | | [SEQ ID: 187] |
| IL-17A | RT-forward | ACTGCTACTGCTGCTGAGCCT |
| | | [SEQ ID: 188] |
| | RT-reverse | GGTGAGGTGGATCGGTTGTAGT |
| | | [SEQ ID: 189] |
| | Taqman-forward | CAATCCCACGAAATCCAGGA |
| | | [SEQ ID: 190] |
| | Taqman-reverse | TTCAGGTTGACCATCACAGTCC |
| | | [SEQ ID: 191] |
| | Probe | CCCAAATTCTGAGGACAAGAACTTCCCC |
| | | [SEQ ID: 192] |

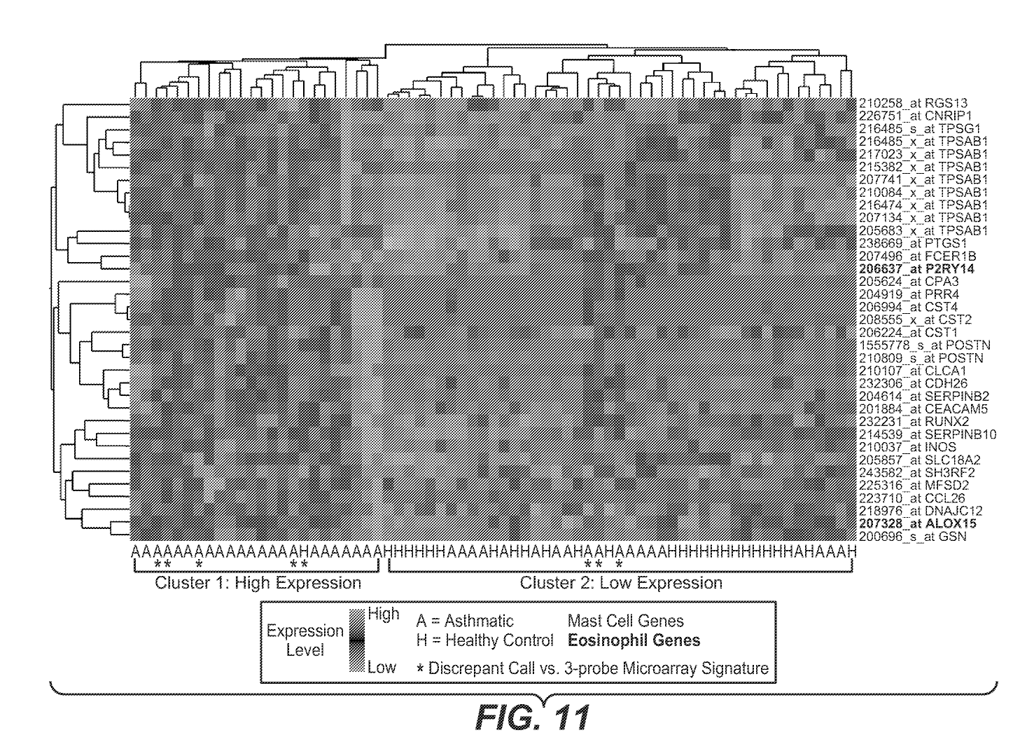

For qPCR for periostin and CEACAM5, relative copy number for periostin and CEACAM5 expression in baseline bronchial epithelial brushing samples were obtained according to a previously described method [45] and log₁₀ transformed. The 35-probe IL13 signature described in Example 9 (see also FIG. 11) was used as a response metric. All models were derived iteratively using the Fit Model platform in JMP 7.0. Ordinal logistic regression was performed to predict response (35 probe IL13 status) having levels (Healthy control; HC)<(IL13 Low)<(IL13 High). The generalized predicative model for probability for each level is described as follows:

$${p}_{HC} = \frac{1}{( 1 + {e}^{( - {β}_{HC} - {β}_{0} )} )}$$

$${p}_{IL 13 low} = \frac{1}{( 1 + {e}^{( - {β}_{IL 13 Low} - {β}_{0} )} )} - {p}_{HC}$$

$${p}_{IL 13 high} = 1 - ( {p}_{HC} + {p}_{IL 13 Low} )$$

$${β}_{0} = {\sum }_{{}_{q}^{}{PCR}{}_{i}^{}}^{k} {A}_{i} \times {X}_{i} ( Linear sum )$$

$${β}_{0} = {\prod }_{{}_{q}^{}{PCR}{}_{i}^{}}^{k} {A}_{i} \times {X}_{i} ( Product for cross terms )$$

$${β}_{x} = intercept estimate of qP C R parameter x$$

Ordinal logistic regression was performed for the following model: (35 probe IL13 status)˜(POSTN)+(CEACAM5). A whole model p-value of <0.0001 was derived from the dataset based on an iterative fit.

### IL 13 Responsive Genes

The relationship between periostin (also known as osteoblast specific factor) (POSTN: 210809_s_at), CLCA1 (also known as chloride channel, calcium activated, family member 1) (CLCA1: 210107_at), and SERPINB2 (also known as serpin peptidase inhibitor, Glade B (ovalbumin), member 2) (SERPINB2: 204614_at) expression level was confirmed using the Wilcoxon Rank Sum test. POSTN expression level was used to categorize baseline asthma samples. A cutoff of 800 units was used, resulting in 21 asthma baseline asthma samples being classified as “IL13 low” (POSTN <800 units) and the remaining 21 samples as “IL13 high” (POSTN >800). Wilcoxon Rank Sum test followed by false discovery rate analysis (qvalue <0.05) [24] identified 35 probes differentially expressed among the two groups. Hierarchical clustering using these probes was undertaken. Due to the presence of many cystatin and serpin family genes in the list differentially regulated probes, additional cystatin and serpin family probes were identified and used in an additional cluster analysis. All statistical analyses were performed using R. Microarray cluster analysis was performed using Cluster and visualized using Java Treeview [25, 26].

### Serum Analyte Assays

Serum IgE was measured by UCSF clinical laboratories or by ELISA using a human serum IgE ELISA kit according to manufacturer's instructions (Bethyl Laboratories). Serum CEA was measured using a human serum CEA ELISA kit according to manufacturer's instructions (Alpco Diagnostics). We developed an electrochemiluminescent assay (ECLA) to measure serum periostin using anti-periostin antibodies (R&D systems). Briefly, monoclonal anti-periostin was coated onto plates at 1.5 micrograms/ml in sodium carbonate buffer, pH 9.6 overnight at 4° C. Plates were blocked in assay buffer (1×PBS pH 7.4, 0.35 M NaCl, 0.5% BSA, 0.05% Tween 20, 0.25% CHAPS, 5 mM EDTA, 15PPM Proclin)+3% BSA for 2 hours at room temperature, then washed 4× with TBST (Tris-buffered saline+0.1% Tween-20). Serum was diluted 1:5 in assay buffer and incubated with agitation at room temperature for 2 h, then washed 4× with TBST. Recombinant periostin (R&D Systems) was used to establish a standard range. Biotinylated polyclonal anti-human periostin (1.5 microgram/ml) (R&D Systems; biotinylated in vitro according to standard methods known in the art) and Ruthenium-streptavidin (0.75 microgram/ml) (Meso Scale Devices) were added in assay buffer+5% goat serum and incubated for 90 minutes at room temperature. Reading buffer (Meso Scale Devices) was added and electrochemiluminescence was read (Meso Scale Devices). Dynamic range was 5-2000 ng/ml.

### Example 2

### IL-4/13 Signature and Subsets of Asthmatics

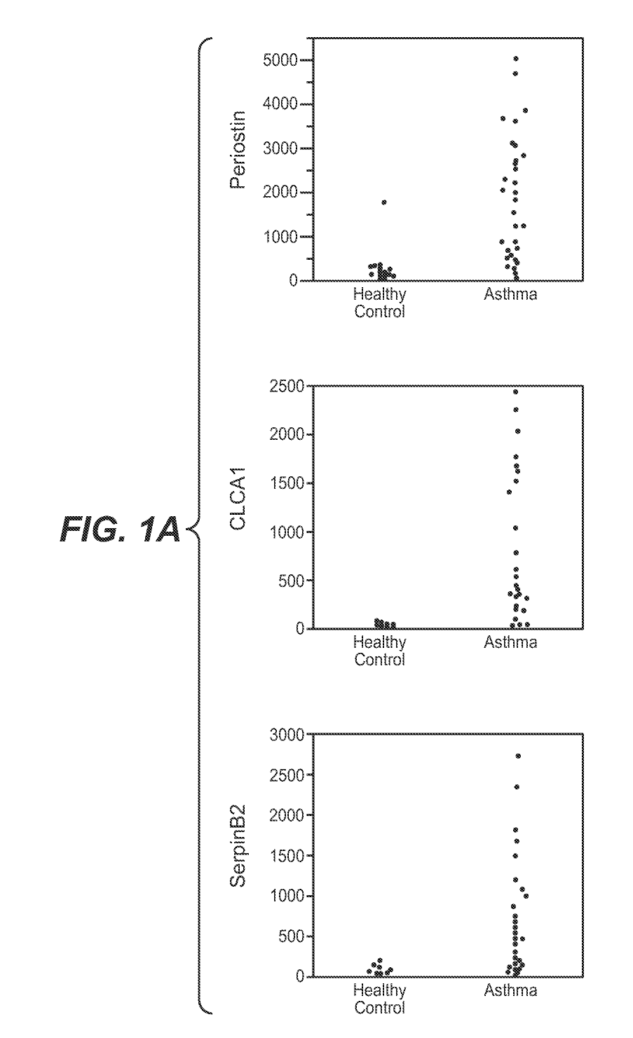

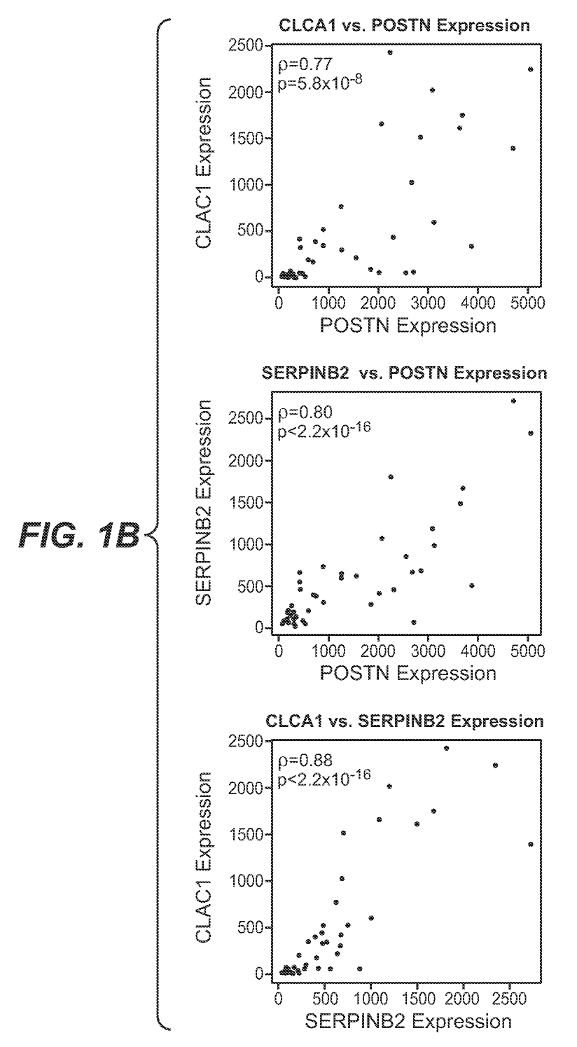

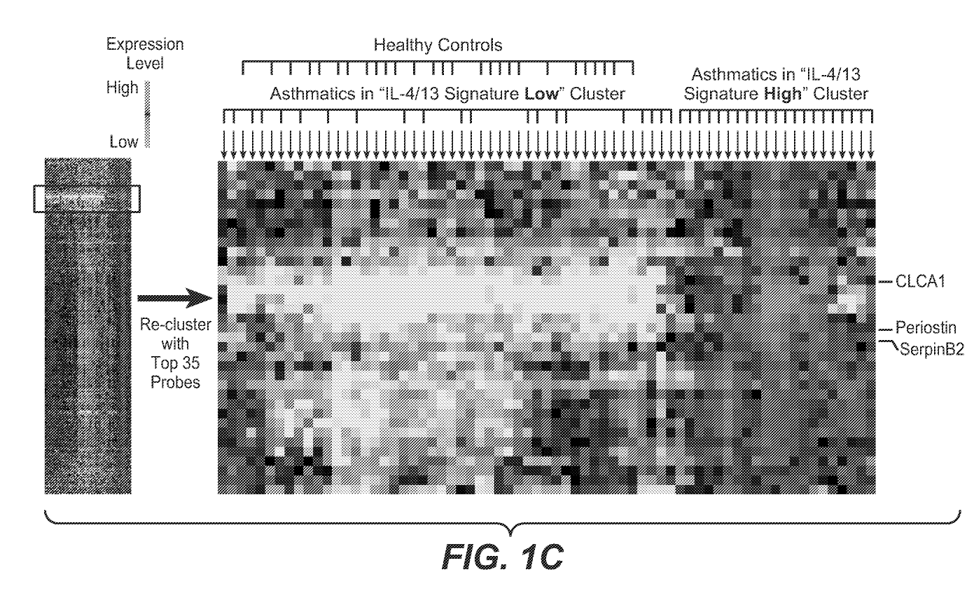

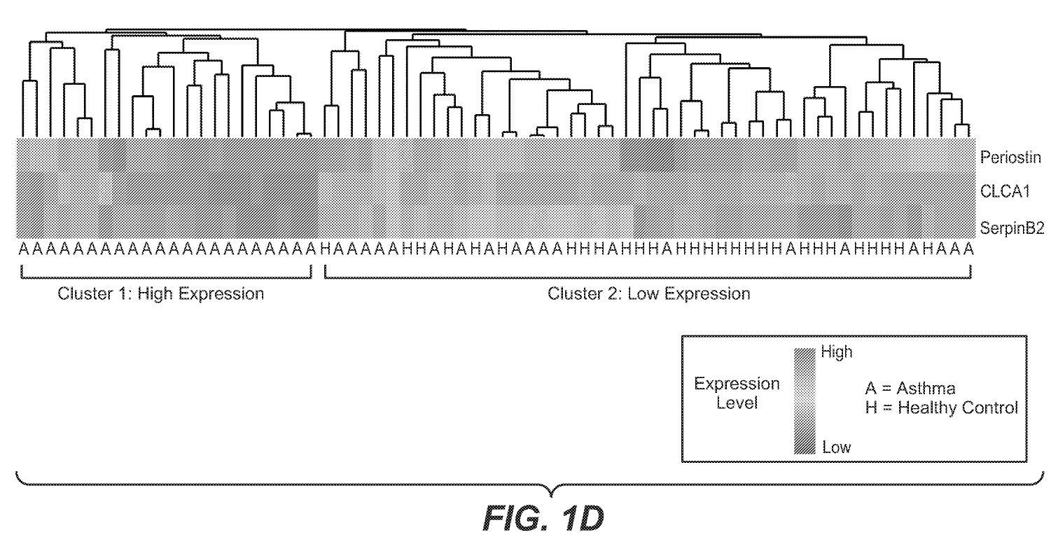

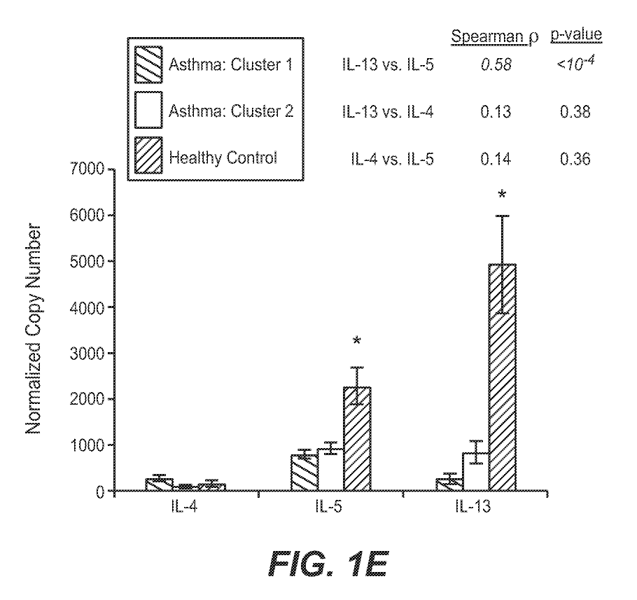

To determine if three IL-13 induced genes (periostin, CLCA1, and serpinB2) reflect a broader pattern of gene expression in asthmatic airway epithelium, we examined whether their expression was co-regulated at baseline within individual subjects among the 42 asthmatics studied. In pairwise comparisons, the expression levels of periostin, CLCA1, and serpinB2 were significantly correlated within individual asthmatics. Furthermore, these genes were highly expressed in some, but not all, of the asthmatic subjects (FIGS. 1A and 1B). In addition, expression levels of these three genes were highly correlated within individual subjects with asthma (FIG. 1B). These data suggest that certain IL-13 markers are over-expressed in a specific subset of patients with asthma. In further experiments, we sought to identify additional genes or markers that might be directly or indirectly regulated by IL-13 and we sought to characterize subsets of asthma patients based on expression of IL-13 markers.

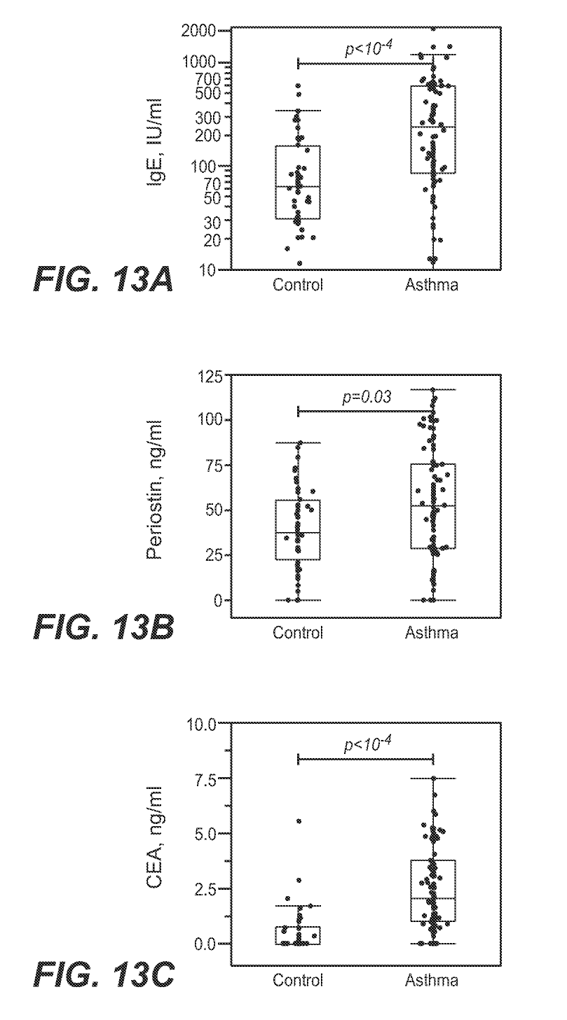

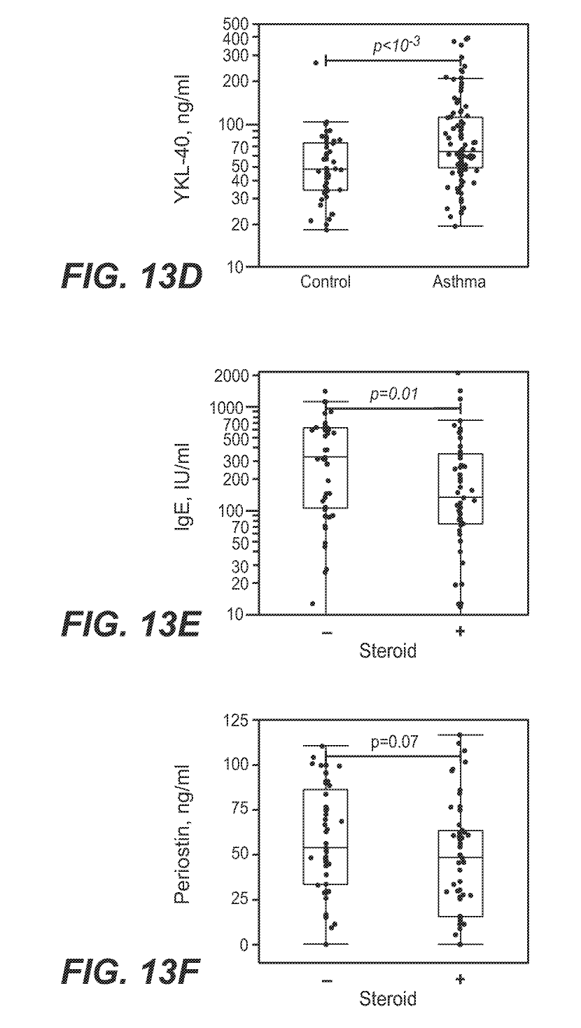

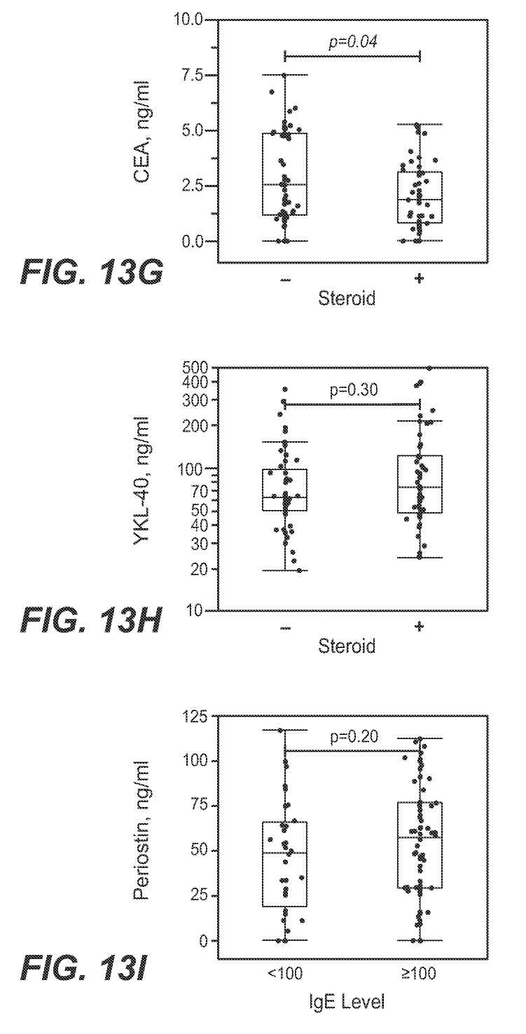

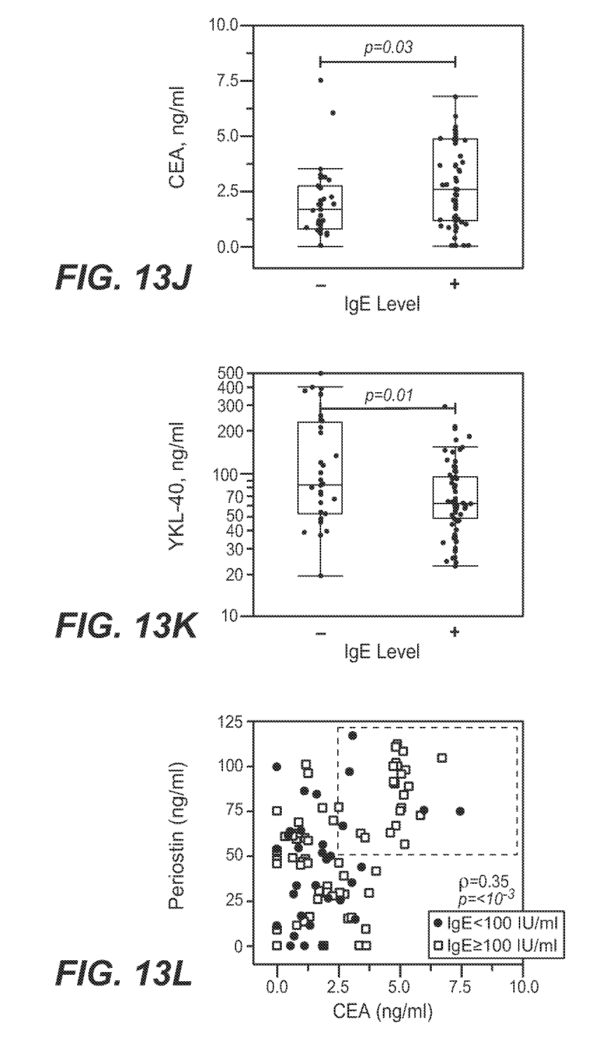

To identify other genes or markers that could potentially be regulated directly or indirectly by IL-13 in asthmatic airway epithelium, we examined the entire microarray dataset across the 42 asthmatic subjects for genes whose expression was significantly correlated with that of periostin. We identified a cluster of 653 probes whose expression was corrugated with periostin in individual subjects below a threshold q-value of 0.05. Unsupervised clustering of all subjects including healthy controls and asthmatics based on expression levels of those 653 probes revealed two major clusters: a cluster with high expression levels of periostin and co-regulated genes and a cluster with low expression levels of periostin and co-regulated genes. The core of this gene cluster (FIG. 1C, right panel) comprises a subset of 35 probes representing the genes shown in FIG. 13, which we refer to herein as “IL-4/13 signature,” “IL-4/13 gene signature,” “IL-13 signature,” or “IL-13 gene signature.” As indicated previously, those terms are used synonymously herein. The cluster with high expression of periostin and co-regulated genes comprised 21 asthmatic subjects and no healthy controls (FIG. 1C, right panel, labeled “Il-4/13 signature high”) whereas the cluster with low expression of periostin and co-regulated genes comprised the remaining 21 asthmatics (FIG. 1C, right panel, labeled “IL-4/13 signature low”) interspersed with all 27 of the healthy controls (FIG. 1C, right panel).

Cluster 1 (“IL-4/13 signature high”) is characterized by high expression levels of the genes corresponding to probes for periostin, CST1, CST2, CST4, CCL26, CLCA1, CDH26, PRR4, serpinB2, serpinB10, CEACAM5, iNOS, C20RF32, PTGS1, P2RY14, RUNX2, SH3RF2, WLRW300, DNAJC12, ALOX15, GSN, RGS13, TGSAB1, PTSG1, FCER1B, and CPA3 and consists of approximately half the asthmatics in the study (N=23 out of 42 asthmatics) and one healthy control out of 27 total healthy controls. Cluster 2 (Healthy controls and “IL-4/13 signature low”) is characterized by low expression levels of the genes corresponding to the indicated probes and consists of the remaining 19 asthmatics and 26/27 healthy controls. Probes corresponding to genes predominantly expressed in mast cells, including RGS13, TPSG1, TPSAB1, FCER1B, CPA3, and SLC18A2 are indicated in blue in Table 2 and probes corresponding to genes predominantly expressed in eosinophils, including P2RY14 and ALOX15 are indicated in orange. Although the epithelial brushings consisted of predominantly epithelial cells and goblet cells (mean 97%, median 98%, minimum 91%), small numbers of infiltrating mast cells and eosinophils were observed in the brushings from cluster 1 asthmatics, and the presence of mast cell and eosinophil genes in the signature likely reflects this infiltration.

To characterize subsets of subjects with asthma based on expression of IL-13 markers, we performed unsupervised hierarchical clustering of all 70 subjects (42 asthmatics and 27 healthy controls) based on the microarray expression levels of periostin, CLCA1, and serpinB2 (FIG. 1D). In this analysis, approximately half of subjects with asthma (N=22) showed consistently high expression levels of IL-13-induced genes and grouped together in one major branch of the cluster dendrogram (cluster 1, the “IL-13 high” subset). Remarkably, although periostin, CLCA1, and serpinB2 were significantly over-expressed when comparing all 42 asthmatics to all 27 healthy controls [8], nearly half of the asthmatics examined in this study (N=20) were indistinguishable from healthy controls on the basis of expression of these three genes. This subset of asthmatics (the “IL-13 low” subset) and all the healthy controls grouped together in the second major branch of the dendrogram (FIG. 1D, cluster 2). Thus, hierarchical clustering based on epithelial gene expression identified two distinct subsets of patients with asthma, referred to herein as “IL-13 high” subset and “IL-13 low” subset.

To confirm the validity of these asthma patient subsets, identified using IL-13 inducible marker expression in epithelial cells, we measured the expression level of IL-13 and certain other Th2 cytokines (i.e. IL-4 and IL-5) in bronchial biopsies obtained contemporaneously from 48 of the subjects (14 healthy controls, 18 cluster 1 asthmatics, and 16 cluster 2 asthmatics). Using qPCR, we found that IL-13, IL-5 and IL-4 expression was detectable in homogenates of bronchial biopsies. Notably, IL-13 and IL-5 expression, but not IL-4 expression, were significantly higher (FIG. 1E, *, p<0.002) in cluster 1 asthmatics compared to cluster 2 asthmatics or healthy controls. There were no significant differences, however, in IL-4, IL-5, or IL-13 expression between asthmatics in cluster 2 and healthy controls (FIG. 1E). In addition, we found that expression levels of IL-13 and IL-5 were highly correlated across all of the subjects with asthma (Spearman's rank order correlation p=0.58, p<0.0001; FIG. 1E). IL-4 shares a dominant signaling pathway with IL-13 and has been shown to induce periostin [7, 9] and CLCA1 [12] expression similarly to IL-13. As elevated levels of IL-4 expressing T cells have been reported in bronchoalveolar lavage (BAL) fluid [79] from asthmatics and we did not specifically examine cytokine gene expression in BAL T cells or cytokine protein levels in BAL or bronchial tissue in this study, we cannot rule out the possibility that the observed induction of periostin, CLCA1, and serpinB2 is due in part to IL-4 as well as to IL-13. Based on the data shown herein, we can confidently discern a correlation between bronchial IL-13 expression and epithelial periostin, CLCA1, and serpinB2 expression. Thus, we use the terms “IL-4/13 high” and “IL-13 high” synonymously to refer to cluster 1 asthmatics and we use the terms “IL-4/13 low” and “IL-13 low” synonymously to refer to cluster 2 asthmatics. It is understood that when the terms “IL-13 high” and “IL-13 low” are used, IL-4 and/or other as yet unidentified factors may also contribute in part to the observed gene expression patterns.

### Example 3

### Constituent Genes of IL-4/13 Signature

Within the IL-4/13 signature, there are two major groups of genes: epithelial or goblet cell expressed genes and mast cell expressed genes. Greater than 90% of cells in each bronchial brushing sample were bronchial epithelial cells or goblet cells (mean 97%, median 98%, minimum 91%). Expression levels of probes corresponding to the following epithelial or goblet cell genes were most significantly co-regulated with those of periostin: CST1, CST2, CCL26, CLCA1, PRR4, serpinB2, CEACAM5, and iNOS (Table 2, indicated with asterisks; >3-fold higher expression in IL-4/13 signature high vs. IL-4/13 signature low subjects). The mouse orthologue of CLCA1, mCLCA3 (also known as gob-5) has been previously identified as a gene associated with goblet cell metaplasia of airway epithelium and mucus production; both are induced by Th2 cytokines including IL-9 and IL-13 [12-14]

TABLE 2

| | | Fold change, | p-value, | q-value, | | IL-4/13 | IL-4/13 | Fold change, | Fold change, |
|---|---|---|---|---|---|---|---|---|---|
| | | High vs. | High vs. | High vs. | Healthy | signature | signature | High vs. | Low vs. |
| Probe | Gene Name | Low | Low | Low | Mean | Low mean | High mean | Control | Control |
| 1555778_a_at | POSTN* | 11.35 | 2.60E−11 | 7.11E−07 | 14.93 | 15.73 | 178.51 | 11.96 | 1.05 |
| 206224_at | CST1* | 11.12 | 9.09E−06 | 0.021609818 | 8.76 | 32.37 | 360.02 | 41.12 | 3.70 |
| 223710_at | CCL26* | 10.22 | 2.88E−05 | 0.045024394 | 6.33 | 3.87 | 39.57 | 6.25 | 0.61 |
| 206994_at | CST1* | 9.98 | 4.90E−06 | 0.014874475 | 10.38 | 67.81 | 676.94 | 65.22 | 6.53 |
| 210107_at | CLCA1* | 9.77 | 1.96E−07 | 0.001785296 | 29.61 | 95.06 | 928.81 | 31.37 | 3.21 |
| 208555_x_at | CST2* | 9.13 | 9.04E−07 | 0.004119975 | 5.13 | 14.75 | 134.71 | 26.26 | 2.88 |
| 210809_s_at | POSTN* | 7.70 | 3.72E−12 | 2.03E−07 | 260.24 | 334.28 | 2572.46 | 9.88 | 1.28 |
| 204919_at | PRR4* | 6.09 | 5.73E−06 | 0.01649484 | 37.33 | 97.20 | 592.05 | 15.86 | 2.60 |
| 207741_x_at | TPSD1 | 4.76 | 1.54E−06 | 0.005250514 | 9.86 | 18.81 | 89.64 | 9.09 | 1.91 |
| 204614_at | SERPINB2* | 4.52 | 4.30E−07 | 0.002615287 | 97.43 | 212.63 | 960.75 | 9.86 | 2.18 |
| 201884_at | CEACAM5* | 3.48 | 4.30E−07 | 0.002615287 | 426.04 | 525.17 | 1830.00 | 4.30 | 1.23 |
| 210037_s_at | | 3.30 | 2.51E−05 | 0.045024394 | 6.39 | 6.54 | 21.60 | 3.38 | 1.02 |
| 216485_s_at | TPSG1 | 3.23 | 7.81E−06 | 0.0203328 | 10.04 | 17.84 | 57.65 | 5.74 | 1.78 |
| 216474_x_at | TPSD1 | 3.06 | 1.60E−07 | 0.00174608 | 46.63 | 77.82 | 238.00 | 5.10 | 1.67 |
| 205683_x_at | TPSD1 | 2.97 | 2.72E−08 | 0.00049597 | 53.99 | 76.74 | 227.95 | 4.22 | 1.42 |
| 225316_at | MFSD2 | 2.96 | 2.18E−05 | 0.042590277 | 29.03 | 26.00 | 76.95 | 2.65 | 0.90 |
| 205624_at | CPA3 | 2.94 | 3.55E−07 | 0.002615287 | 99.69 | 166.29 | 489.17 | 4.91 | 1.67 |
| 206637_at | GPR105 | 2.88 | 6.27E−07 | 0.003117623 | 40.90 | 65.93 | 189.66 | 4.64 | 1.61 |
| 232306_at | CDH26 | 2.85 | 1.29E−06 | 0.00470447 | 223.92 | 326.79 | 932.02 | 4.16 | 1.46 |
| 207134_x_at | TPSD1 | 2.66 | 6.27E−07 | 0.003117623 | 50.28 | 86.08 | 228.78 | 4.55 | 1.71 |
| 210084_x_at | TPSD1 | 2.56 | 6.70E−06 | 0.018307462 | 48.91 | 77.59 | 198.54 | 4.06 | 1.59 |
| 200696_s_at | GSN | 2.50 | 2.88E−05 | 0.045024394 | 246.87 | 224.72 | 562.74 | 2.28 | 0.91 |
| 226751_at | C2ORF32 | 2.50 | 9.09E−06 | 0.021609818 | 35.39 | 32.96 | 82.47 | 2.33 | 0.93 |
| 238429_at | TRACH2000196 | 2.39 | 2.88E−05 | 0.045024394 | 36.79 | 37.68 | 89.91 | 2.44 | 1.02 |
| 218976_at | DNAJC12 | 2.32 | 2.18E−05 | 0.042590277 | 48.54 | 38.92 | 90.11 | 1.86 | 0.80 |
| 217023_x_at | TPSD1 | 2.31 | 1.29E−06 | 0.00470447 | 53.13 | 67.76 | 156.32 | 2.94 | 1.28 |
| 215382_x_at | TPSD1 | 2.30 | 1.08E−06 | 0.004549197 | 38.96 | 52.95 | 121.86 | 3.13 | 1.36 |
| 210258_at | RGS13 | 2.25 | 2.88E−05 | 0.045024394 | 8.75 | 7.56 | 17.04 | 1.95 | 0.86 |
| 205857_at | SLC18A2 | 2.23 | 1.05E−07 | 0.001435039 | 84.08 | 100.96 | 225.07 | 2.68 | 1.20 |
| 214539_at | SERPINB10 | 2.15 | 1.06E−05 | 0.024063758 | 42.37 | 40.04 | 86.16 | 2.03 | 0.95 |
| 243582_at | SH3RF2 | 2.00 | 1.89E−05 | 0.039805857 | 82.64 | 85.89 | 171.80 | 2.08 | 1.04 |
| 207496_at | FCER1B | 1.92 | 2.51E−05 | 0.045024394 | 37.55 | 37.03 | 71.28 | 1.90 | 0.99 |
| 232231_at | RUNX2 | 1.77 | 2.88E−05 | 0.045024394 | 288.42 | 299.21 | 529.59 | 1.84 | 1.04 |
| 238669_at | PTGS1 | 1.73 | 4.18E−06 | 0.013429003 | 82.69 | 88.43 | 152.98 | 1.85 | 1.07 |
| 207328_at | ALOX15 | 1.72 | 1.64E−05 | 0.03586964 | 812.57 | 895.94 | 1538.53 | 1.89 | 1.10 |

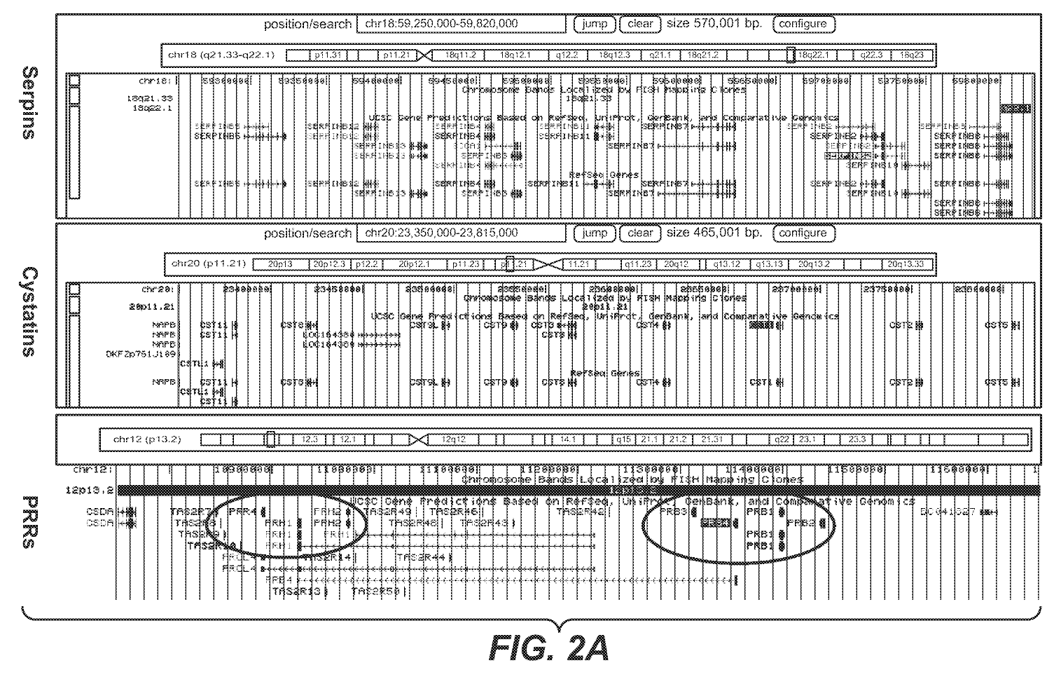

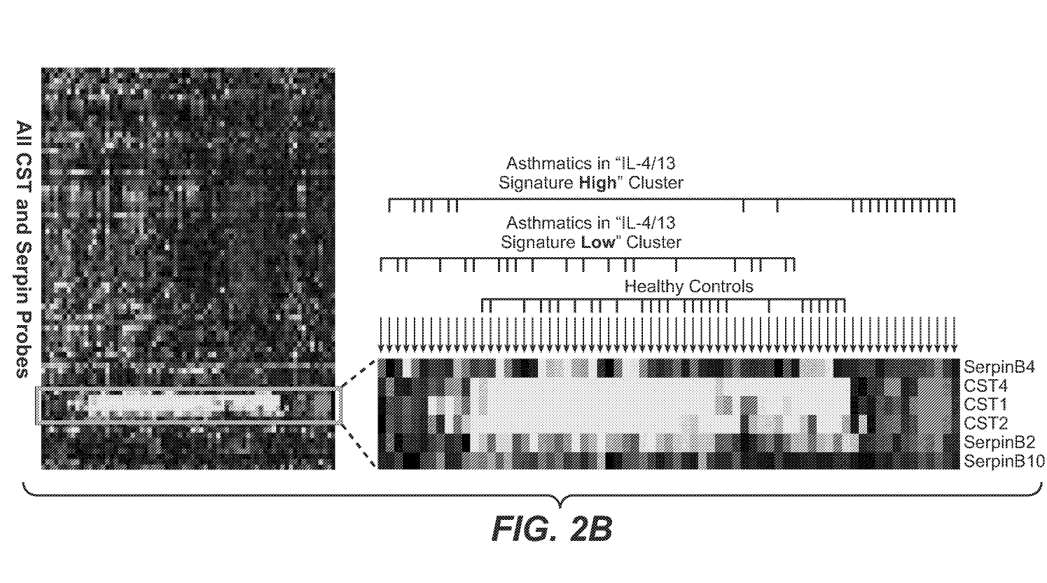

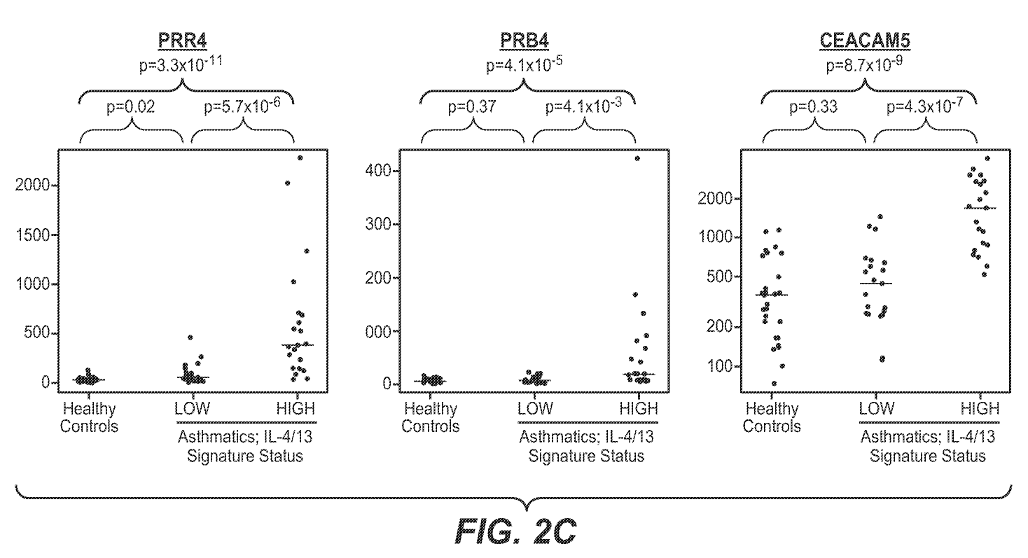

SerpinB2 is a member of a large family of serine protease inhibitors encoded in a gene cluster on chromosome 18q21 (FIG. 2A, top; screen capture from UCSC Genome Browser at http://genome.ucsc.edu). Expression levels of serpins B2 [8], B3, and B4 are induced in airway epithelial cells upon stimulation by recombinant IL-4 and IL-13 [7, 15].

Cystatins (CST) 1 and 2 are members of a large family of cysteine protease inhibitors encoded in a gene cluster on chromosome 20p11 (FIG. 2A, middle; screen capture from UCSC Genome Browser at http://genome.ucsc.edu). Several cystatins are expressed in bronchial epithelium [16]; CST4 has been identified at elevated levels in bronchoalveolar lavage fluid (BAL) of asthmatics [17]; serum CST3 is elevated in asthmatics relative to healthy controls and its levels are decreased by ICS treatment [18]. As serpin and CST gene families are each colocalized on the chromosome, we explored whether any additional members of the serpin and cystatin gene families are co-regulated with those already identified. We performed unsupervised clustering of the microarray data, restricted to serpin and cystatin gene families. We found that serpins B2, B4, and B10; and cystatins 1, 2, and 4 were significantly co-regulated, with the highest expression levels occurring in asthmatics positive for the “IL-4/13 signature” (FIG. 2B).

PRR4 is a member of a large family of proteins encoded in a gene cluster on chromosome 12p13 (FIG. 2A, bottom; screen capture from UCSC Genome Browser at http://genome.ucsc.edu). These proline-rich proteins are found in mucosal secretions including saliva and tears. Related, but non-orthologous proteins SPRR1a, 2a, and 2b have been identified in bronchial epithelium in a mouse model of asthma and are induced by IL-13 [19, 20]. Proline-rich proteins from the PRR/PRB family have been identified in bronchial secretions [21] and their expression has been documented in bronchial epithelium [16]. Of the PRR/PRB family, PRR4 and PRB4 were significantly upregulated in asthmatics with high expression of the IL-4/13 gene signature (FIG. 2C, left and middle).

CCL26 (Eotaxin-3) is an IL-4 and IL-13 inducible chemokine in asthmatic airway epithelium.

CEACAM5 encodes a cell-surface glycoprotein found in many epithelial tissues and elevated serum. CEACAM5 (carcinoembryonic antigen; CEA) is a well-documented systemic biomarker of epithelial malignancies and metastatic disease. Elevated CEA levels have been reported in a subset of asthmatics, with particularly high serum levels observed in asthmatics with mucoid impaction [22]. CEACAM5 is significantly upregulated in IL-4/13 signature high asthmatic airway epithelium compared to IL-4/13 signature low and healthy control airway epithelium (FIG. 2C, right), which suggests that serum CEA levels may be used to distinguish between these two asthmatic sub-phenotypes.

Inducible nitric oxide synthase (iNOS) is associated with airway inflammation and is induced by IL-13 in human primary bronchial epithelial cell cultures [23]. The measurement of exhaled nitric oxide (eNO), a product of iNOS enzymatic activity, is commonly used in the diagnosis and monitoring of asthma.

### Example 4

### Mast Cells

Although the airway brushings used in this study comprised predominantly epithelial and goblet cells, there were small but significant percentages of infiltrating leukocytes in many of the samples. Genes whose expression is specific to mast cells, including tryptases (TPSD1, TPSG1), caboxypeptidase A3 (CPA3), and FcepsilonRlbeta, were significantly correlated with the IL-4/13 gene signature (Table 2 and Table 4, mast cell genes marked with double astericks in Table 4). Given the significant role of tissue-resident mast cells in allergic disease and the recent observation that the presence of IL-13 expressing mast cells in asthmatic endobronchial biopsy specimens is positively correlated with detectable levels of IL-13 in sputum [6], the high correlation between mast cell-specific genes and the IL-4/13 signature suggests that: 1) mast cells may be a significant source of IL-13 in the airway epithelium and 2) mast cell infiltration into airway epithelium may be a unique feature of the IL-4/13 signature high subset of asthmatics.

### Example 5

### Combinations that Predict IL-4/13 Signature

Expression levels of individual genes in the IL-4/13 signature may predict the IL-4/13 signature status of individual subjects with variable accuracy; however combinations of these genes may be used to assign individual subjects to the IL-4/13 signature high or low category with increased sensitivity and specificity.

### Example 6

### Steroid Effect

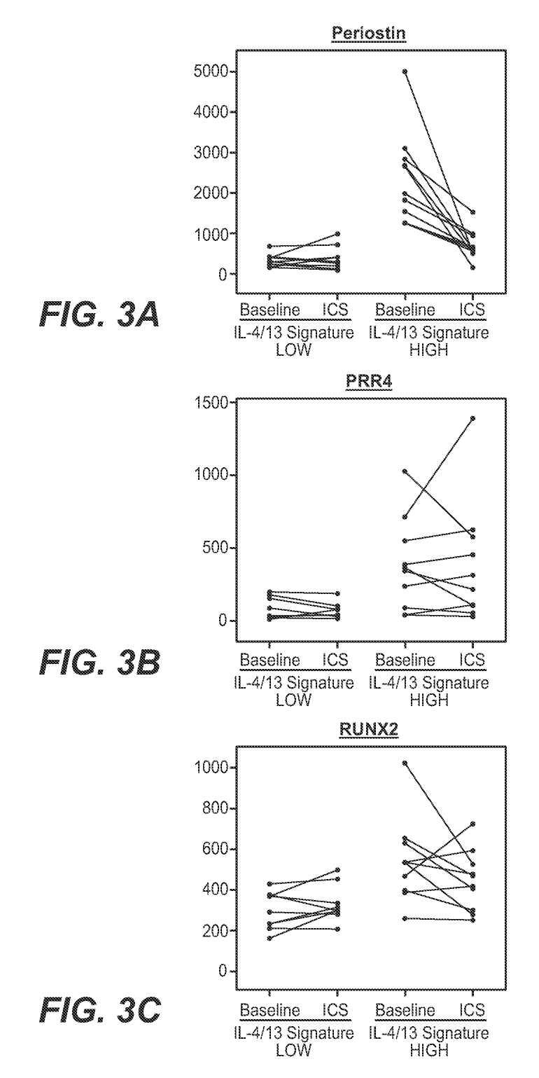

The standard of care for bronchial asthma that is not well-controlled on symptomatic therapy (i.e. beta-adrenergic agonists) is inhaled corticosteroids (ICS). In mild-to-moderate asthmatics with elevated levels of IL-13 in the airway [6] and in eosinophilic esophagitis patients with elevated expression levels of IL-13 in esophageal tissue [11], ICS treatment substantially reduces the level of IL-13 and IL-13-induced genes in the affected tissues. In airway epithelium of asthmatics after one week of ICS treatment and in cultured bronchial epithelial cells, we have shown that corticosteroid treatment substantially reduces IL-13-induced expression levels of periostin, serpinB2, and CLCA1 [8]. Further examination of the genes listed in Table 2 revealed that, in the 19 subjects in our study who received one week of ICS treatment prior to a second bronchoscopy, the vast majority of IL-4/13 signature genes was significantly downregulated by ICS treatment in asthmatic bronchial airway epithelium (periostin shown as an example, FIG. 3A). This downregulation could be the result of ICS-mediated reduction of IL-13 levels, ICS-mediated reduction of target gene expression, or a combination of the two. However, two genes in the IL-4/13 signature, PRR4 (FIG. 3B) and RUNX2 (FIG. 3C), were not substantially downregulated in individual subjects after one week of ICS treatment. This suggests that PRR4 and RUNX2 may be steroid-insensitive markers of the IL-4/13 signature in asthmatic airway epithelium. Another possibility is that PRR4 and RUNX2 are only indirectly regulated by IL-4 and/or IL-13; for example, as PRR4 is found in many secretions, it may be a goblet cell-specific gene. As goblet cell differentiation from epithelial cells is induced by IL-13, ICS-mediated inhibition of IL-13 and IL-13 dependent processes may not substantially impact on goblet cell number after only 7 days of treatment, but after longer-term ICS treatment, goblet cell numbers (and hence PRR4 expression in endobronchial brushings) may be expected to decrease. In severe asthmatics who are refractory to ICS treatment, a similar fraction of subjects (approximately 40%) was found to have detectable sputum IL-13 levels to that seen in mild, ICS-naïve asthmatics [6], which is consistent with the fraction of subjects with the IL-4/13 signature observed in this study. This observation suggests that, although the IL-4/13 signature is significantly downregulated by ICS treatment in the mild-moderate, ICS-responsive asthmatics examined in the present study, it may still be present in severe steroid-resistant asthmatics.

### Example 7

### Relationship of IL-4/13 Signature to Clinical Features and Other Biomarkers

Demographics

Eosinophilic asthma, as defined by elevated levels of airway eosinophils, is associated with atopy and occurs with approximately equal prevalence between males and females, while the non-eosinophilic phenotype, as defined by a relative absence of eosinophils in the airway and associated with a lack of atopy, shows a significant female predominance [1]. Of the subjects classified according to the airway epithelial IL-4/13 gene signature, 10/21 (48%) IL-4/13 signature high subjects were female while 15/21 (71%) IL-4/13 signature low subjects were female (Table 3). There was no significant skewing by self-reported ethnicity between the IL-4/13 signature low and high groups.

Gender distribution of IL-4/13 signature, N (%)

| Category | F | M |
|---|---|---|
| LOW | 15/(71) | 6(29) |
| HIGH | 10(48) | 11(52) |
| CONTROL | 15(54) | 13(46) |

FEV₁ and Methacholine Responsiveness

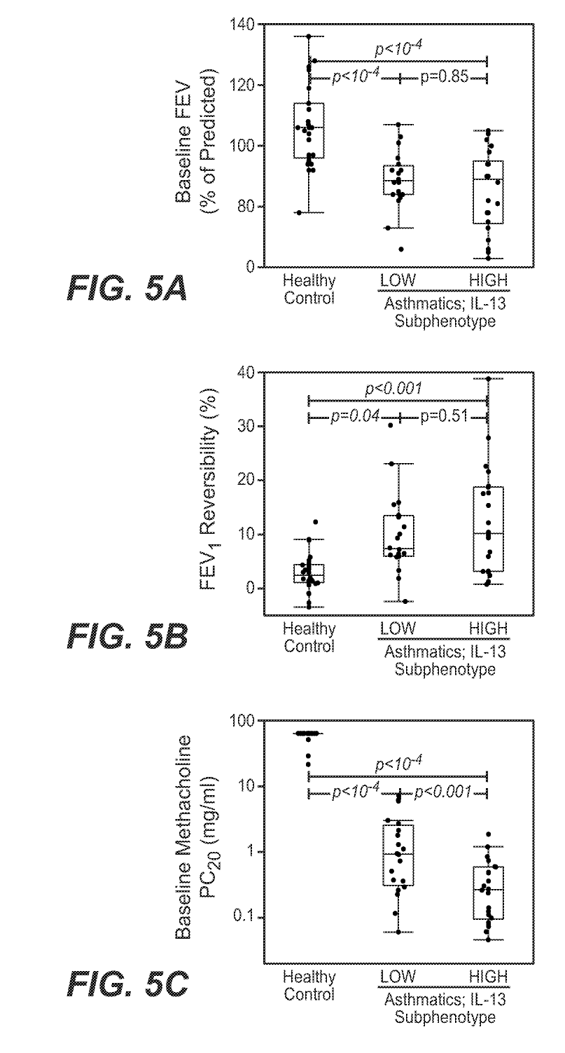

While the gender skewing between the IL-4/13 low and high groups suggest that the observed gene expression patterns in asthmatic airway epithelium reflect stable underlying phenotypes, it is possible that the observed gene expression patterns merely reflect disease severity or activity at the time of bronchoscopy. To determine whether the IL-4/13 signature was correlated to asthma severity, we compared forced expiratory volume in one second (FEV₁, as a percentage of predicted from patient weight, measured at a screening visit one week prior to bronchoscopy) between the groups and found that, while both the IL-4/13 signature high and low groups had significantly lower FEV₁ than healthy controls, there was no statistically significant difference between the groups (see FIG. 5A), although there were more subjects that might be classified as “moderate” (i.e. FEV₁ 60-80% predicted) in the IL-4/13 signature high group than in the low group. The minimal concentration of methacholine in mg/ml required to induce a decrease in FEV₁ of 20% (PC₂₀, measured at a screening visit one week prior to bronchoscopy) is a measure of bronchial hyperresponsiveness. This is a measure of bronchial hyper-reactivity (BHR). Both the IL-4/13 signature high and low groups had significantly lower PC₂₀ values than healthy controls; while there was a trend toward lower PC₂₀ values in the IL-4/13 signature high group than in the low group, this difference did not reach statistical significance (see FIG. 5C).

IgE and Eosinophils (Peripheral and Airway)

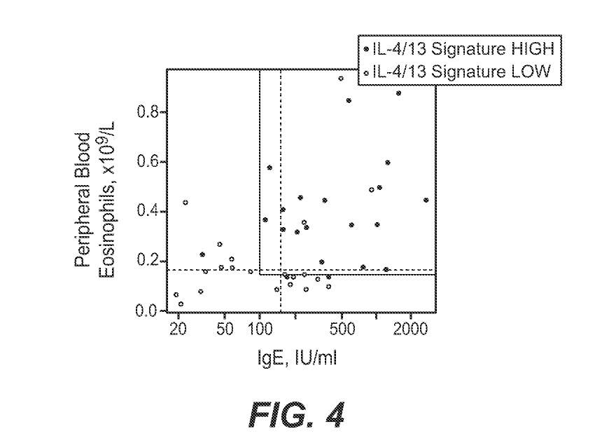

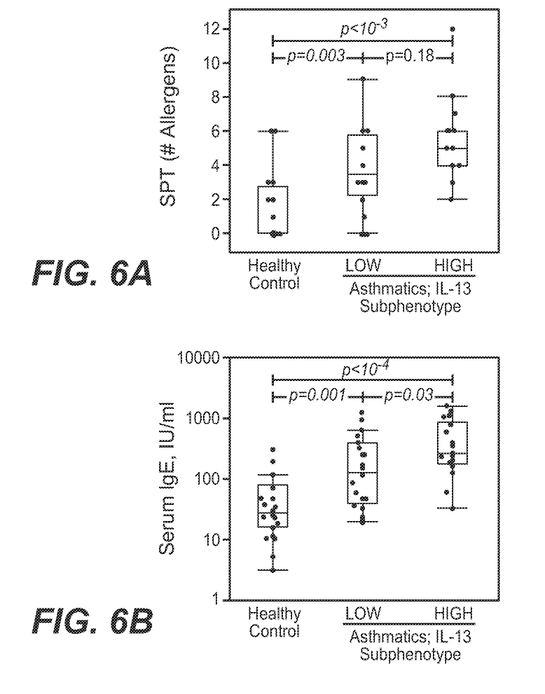

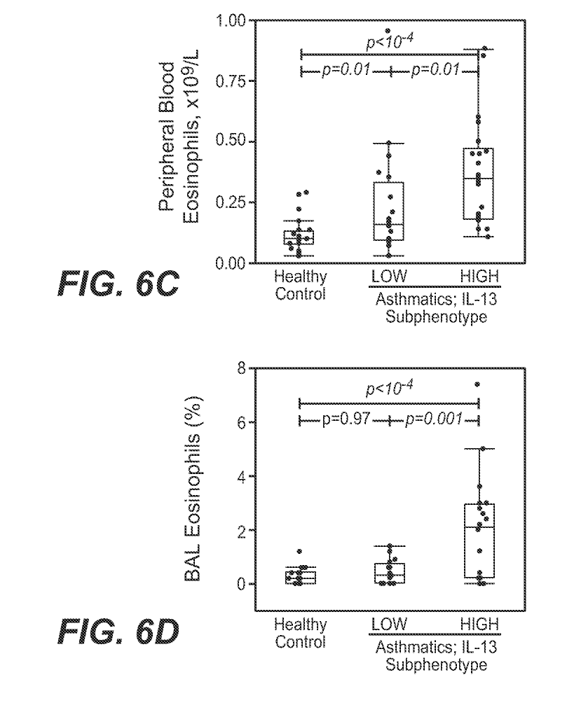

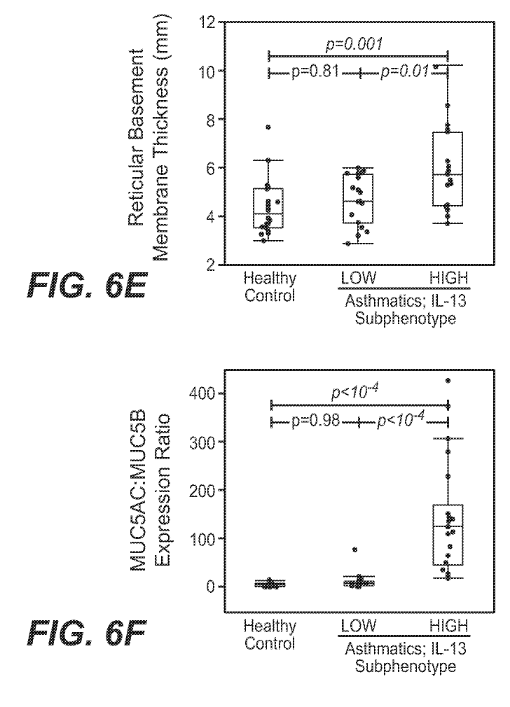

To determine whether the IL-4/13 signature status of an individual subject could be predicted by standard measures of atopy, we examined levels of serum IgE (international units per milliliter; 1 IU=2.4 ng), peripheral blood eosinophil counts (absolute number of eosinophils×10̂9 per liter of blood), and eosinophil percentages in bronchoalveolar lavage fluid (BAL) (percentage of eosinophils relative to the total number of non-squamous cells in bronchoalveolar lavage fluid) using standard clinical laboratory tests, obtained at the time of bronchoscopy. When subjects were stratified for IL-4/13 signature status, there were significant differences in serum IgE (see FIG. 6B), peripheral blood eosinophil counts (see FIG. 6C), and BAL eosinophil percentage (see FIG. 6D), with significantly higher values for each analyte observed in the IL-4/13 signature high group relative to the low group. Taken individually, neither IgE level nor peripheral blood eosinophil count predicts the airway epithelial IL-4/13 signature status of any individual subject with simultaneously high sensitivity and specificity. However, among individual asthmatics, IgE level and peripheral blood eosinophil counts are weakly but significantly correlated (rho=0.44, p=3.4×10⁻³). When considered as a composite, empirically derived cutoff values of both 100 IU/ml IgE and 0.14×10⁹/L eosinophils predict the airway epithelial IL-4/13 signature status of individual subjects with high sensitivity and specificity (FIG. 4; 18/21 correct for both low and high IL-4/13 signature; sensitivity=86%, specificity=86%).

TABLE 4
IL-4/13 gene signature
genes and exemplary probes.

| Gene | Example Probes |
|---|---|
| POSTN | 1555778_a_at: |
| | AAAGAATCTGACATCATGACAACAAATGGTGTAATTCATG |
| | TTGTAGATAAACTCCTCTATCCAGCAGACACACCTGTTGG |
| | AAATGATCAACTGCTGGAAATACTTAATAAATTAATCAAA |
| | TACATCCAAATTAAGTTTGTTCGTGGTAGCACCTTCAAAG |
| | AAATCCCCGTGACTGTCTATAGACCCACACTAACAAAAGT |
| | CAAAATTGAAGGTGAACCTGAATTCAGACTGATTAAAGAA |
| | GGTGAAACAATAACTGAAGTGATCCATGGAGAGCCAATTA |
| | TTAAAAAATACACCAAAATCATTGATGGAGTGCCTGTGGA |
| | AATAACTGAAAAAGAGACACGAGAAGAACGAATCATTACA |
| | GGTCCTGAAATAAAATACACTAGGATTTCTACTGGAGGTG |
| | GAGAAACAGAAGAAACTCTGAAGAAATTGTTACAAGAAGA |
| | AGACACACCCGTGAGGAAGTTGCAAGCCAACAAAAAAGTT |
| | CAANGGATCTAGAAGACGATTAAGGGAAGGTCGTTCTCAG |
| | TGAAAATCCA |
| | [SEQ ID NO: 31] |
| | 210809_s_at: |
| | AAATTGTGGAGTTAGCCTCCTGTGGAGTTAGCCTCCTGTG |
| | GTAAAGGAATTGAAGAAAATATAACACCTTACACCCTTTT |
| | TCATCTTGACATTAAAAGTTCTGGCTAACTTTGGAATCCA |
| | TTAGAGAAAAATCCTTGTCACCAGATTCATTACAATTCAA |
| | ATCGAAGAGTTGTGAACTGTTATCCCATTGAAAAGACCGA |
| | GCCTTGTATGTATGTTATGGATACATAAAATGCACGCAAG |
| | CCATTATCTCTCCATGGGAAGCTAAGTTATAAAAATAGGT |
| | GCTTGGTGTACAAAACTTTTTATATCAAAAGGCTTTGCAC |
| | ATTTCTATATGAGTGGGTTTACTGGTAAATTATGTTATTT |
| | TTTACAACTAATTTTGTACTCTCAGAATGTTTGTCATATG |
| | CTTCTTGCAATGC |
| | [SEQ ID NO: 32] |
| CST1 | 206994_at: |
| | GCGAGTACAACAAGGCCACCGAAGATGAGTACTACAGACG |
| | CCCGCTGCAGGTGCTGCGAGCCAGGGAGCAGACCTTTGGG |
| | GGGGTGAATTACTTCTTCGACGTAGAGGTGGGCCGCACCA |
| | TATGTACCAAGTCCCAGCCCAACTTGGACACCTGTGCCTT |
| | CCATGAACAGCCAGAACTGCAGAAGAAACAGTTATGCTCT |
| | TTCGAGATCTACGAAGTTCCCTGGGAGGACAGAATGTCCC |
| | TGGTGAATTCCAGGTGTCAAGAAGCCTAGGGGTCTGTGCC |
| | AGGCCAGTCACACCGACCACCACCCACTCCCACCCCCTGT |
| | AGTGCTCCCACCCCTGGACTGGTGGCCCCCACCCTGCGGG |
| | AGGCCTCCCCATGTGCCTGTGCCAAGAGACAGACAGAGAA |
| | GGCTGCAGGAGTCCTTTGTTGCTCAGCAGGGCGCTCTGCC |
| | CTCCCTCCTTCCTTCTTGCTTCTAATAGACCTGGTACATG |
| | GTACACACACCCC |
| | [SEQ ID NO: 33] |
| | 206224_at: |
| | GGAGGATAGGATAATCCCGGGTGGCATCTATAACGCAGAC |
| | CTCAATGATGAGTGGGTACAGCGTGCCCTTCACTTCGCCA |
| | TCAGCGAGTATAACAAGGCCACCAAAGATGACTACTACAG |
| | ACGTCCGCTGCGGGTACTAAGAGCCAGGCAACAGACCGTT |
| | GGGGGGGTGAATTACTTCTTCGACGTAGAGGTGGGCCGAA |
| | CCATATGTACCAAGTCCCAGCCCAACTTGGACACCTGTGC |
| | CTTCCATGAACAGCCAGAACTGCAGAAGAAACAGTTGTGC |
| | TCTTTCGAGATCTACGAAGTTCCCTGGGAGAACAGAAGGT |
| | CCCTGGTGAAATCCAGGTGTCAAGAATCCTAGGGATCTGT |
| | GCCAG |
| | [SEQ ID NO: 34] |
| CCL26 | 223710_at: |
| | GAGAAGGGCCTGATTTGCAGCATCATGATGGGCCTCTCCT |
| | TGGCCTCTGCTGTGCTCCTGGCCTCCCTCCTGAGTCTCCA |
| | CCTTGGAACTGCCACACGTGGGAGTGACATATCCAAGACC |
| | TGCTGCTTCCAATACAGCCACAAGCCCCTTCCCTGGACCT |
| | GGGTGCGAAGCTATGAATTCACCAGTAACAGCTGCTCCCA |
| | GCGGGCTGTGATATTCACTACCAAAAGAGGCAAGAAAGTC |
| | TGTACCCATCCAAGGAAAAAATGGGTGCAAAAATACATTT |
| | CTTTACTGAAAACTCCGAAACAATTGTGACTCAGCTGAAT |
| | TTTCATCCGAGGACGCTTGGACCCCGCTCTTGGCTCTGCA |
| | GCCCTCTGGGGAGCCTGCGGAATCTTTTCTGAAGGCTACA |
| | TGGACCCGCT |
| | [SEQ ID NO: 35] |
| CLCA1 | 210107_at: |
| | GGCCAAATCACCGACCTGAAGGCGGAAATTCACGGGGGCA |
| | GTCTCATTAATCTGACTTGGACAGCTCCTGGGGATGATTA |
| | TGACCATGGAACAGCTCACAAGTATATCATTCGAATAAGT |
| | ACAAGTATTCTTGATCTCAGAGACAAGTTCAATGAATCTC |
| | TTCAAGTGAATACTACTGCTCTCATCCCAAAGGAAGCCAA |
| | CTCTGAGGAAGTCTTTTTGTTTAAACCAGAAAACATTACT |
| | TTTGAAAATGGCACAGATCTTTTCATTGCTATTCAGGCTG |
| | TTGATAAGGTCGATCTGAAATCAGAAATATCCAACATTGC |
| | ACGAGTATCTTTGTTTATTCCTCCACAGACTCCGCCAGAG |
| | ACACCTAGTCCTGATGAAACGTCTGCTCCTTGTCCTAATA |
| | TTCATATCAACAGCACCATTCCTGGCATTCACATTTTAAA |
| | AATTATGTGGAAGTGGATAGGAGAACTGCAGCTGTCAATA |
| | GCCTAGGGC |
| | [SEQ ID NO: 36] |
| CST2 | 208555_x_at: |
| | GAGCCCCCAGGAGGAGGACAGGATAATCGAGGGTGGCATC |
| | TATGATGCAGACCTCAATGATGAGCGGGTACAGCGTGCCC |
| | TTCACTTTGTCATCAGCGAGTATAACAAGGCCACTGAAGA |
| | TGAGTACTACAGACGCCTGCTGCGGGTGCTACGAGCCAGG |
| | GAGCAGATCGTGGGCGGGGTGAATTACTTCTTCGACATAG |
| | AGGTGGGCCGAACCATATGTACCAAGTCCCAGCCCAACTT |
| | GGACACCTGTGCCTTCCATGAACAGCCAGAACTGCAGAAG |
| | AAACAGTTGTGCTCTTTCCAGATCTACGAAGTTCCCTGGG |
| | AGGA |
| | [SEQ ID NO: 37] |
| PRR4 | 204919_at: |
| | AAGACTTTACTTTCACCATACCAGATGTAGAGGACTCAAG |
| | TCAGAGACCAGATCAGGGACCCCAGAGACCTCCTCCTGAA |
| | GGACTCCTACCTAGACCCCCTGGTGATAGTGGTAACCAAG |
| | ATGATGGTCCTCAGCAGAGACCACCAAAACCAGGAGGCCA |
| | TCACCGCCATCCTCCCCCACCTCCTTTTCAAAATCAGCAA |
| | CGACCACCCCAACGAGGACACCGTCAACTCTCTCTACCCC |
| | GATTTCCTTCTGTCAGCCTGCAGGAAGCATCATCATTCTT |
| | CCGGAGGGACAGACCAGCAAGACATCCCCA |
| | [SEQ ID NO: 38] |
| SERPINB2 | Serpin peptidase inhibitor, |
| | clade B (ovalbumin), member 2 |
| | 204614_at: |
| | TTCCTCACCCTAAAACTAAGCGTGCTGCTTCTGCAAAAGA |
| | TTTTTGTAGATGAGCTGTGTGCCTCAGAATTGCTATTTCA |
| | AATTGCCAAAAATTTAGAGATGTTTTCTACATATTTCTGC |
| | TCTTCTGAACAACTTCTGCTACCCACTAAATAAAAACACA |
| | GAAATAATTAGACAATTGTCTATTATAACATGACAACCCT |
| | ATTAATCATTTGGTCTTCTAAAATGGGATCATGCCCATTT |
| | AGATTTTCCTTACTATCAGTTTATTTTTATAACATTAACT |
| | TTTACTTTGTTATTTATTATTTTATATAATGGTGAGTTTT |
| | TAAATTATTGCTCACTGCCTATTTAATGTAGCTAATAAAG |
| | TTATAGAAGCAGATGATCTGTTAATTTCCTATCTAATAAA |
| | TGCCTTTAATTGTTCTCATAATGAAGAATAAGTAGGTACC |
| | CTCCATGCCCTTCTGTAATAAATAT |
| | [SEQ ID NO: 39] |
| CEACAM5 | 201884_at: |
| | AGAAGACTCTGACCTGTACTCTTGAATACAAGTTTCTGAT |
| | ACCACTGCACTGTCTGAGAATTTCCAAAACTTTAATGAAC |
| | TAACTGACAGCTTCATGAAACTGTCCACCAAGATCAAGCA |
| | GAGAAAATAATTAATTTCATGGGACTAAATGAACTAATGA |
| | GGATTGCTGATTCTTTAAATGTCTTGTTTCCCAGATTTCA |
| | GGAAACTTTTTTTCTTTTAAGCTATCCACTCTTACAGCAA |
| | TTTGATAAAATATACTTTTGTGAACAAAAATTGAGACATT |
| | TACATTTTCTCCCTATGTGGTCGCTCCAGACTTGGGAAAC |
| | TAT |
| | [SEQ ID NO: 40] |
| iNOS | Inducible nitric oxide synthase |
| | 210037_s_at: |
| | TCATCGGGCCTGGCACAGGCATCGCGCCCTTCCGCAGTTT |
| | CTGGCAGCAACGGCTCCATGACTCCCAGCACAAGGGAGTG |
| | CGGGGAGGCCGCATGACCTTGGTGTTTGGGTGCCGCCGCC |
| | CAGATGAGGACCACATCTACCAGGAGGAGATGCTGGAGAT |
| | GGCCCAGAAGGGGGTGCTGCATGCGGTGCACACAGCCTAT |
| | TCCCGCCTGCCTGGCAAGCCCAAGGTCTATGTTCAGGACA |
| | TCCTGCGGCAGCAGCTGGCCAGCGAGGTGCTCCGTGTGCT |
| | CCACAAGGAGCCAGGCCACCTCTATGTTTGCGGGGATGTG |
| | CGCATGGCCCGGGACGTGGCCCACACCCTGAAGCAGCTGG |
| | TGGCTGCCAAGCTGAAATTGAATGAGGAGCAGGTCGAGGA |
| | CTATTTCTTTCAGCTCAAGAGCCAGAAGCGCTATCACGAA |
| | GATATCTTTGGTGCTGTATTTCCTTACGAGGCGAAGAAGG |
| | ACAGGGTGGCGGTGCAGCCC |
| | [SEQ ID NO: 41] |
| SERPINB4 | 210413_x_at: |
| | GTCGATTTACACTTACCTCGGTTCAAAATGGAAGAGAGCT |
| | ATGACCTCAAGGACACGTTGAGAACCATGGGAATGGTGAA |
| | TATCTTCAATGGGGATGCAGACCTCTCAGGCATGACCTGG |
| | AGCCACGGTCTCTCAGTATCTAAAGTCCTACACAAGGCCT |
| | TTGTGGAGGTCACTGAGGAGGGAGTGGAAGCTGCAGCTGC |
| | CACCGCTGTAGTAGTAGTCGAATTATCATCTCCTTCAACT |
| | AATGAAGAGTTCTGTTGTAATCACCCTTTCCTATTCTTCA |
| | TAAGGCAAAATAAGACCAACAGCATCCTCTTCTATGGCAG |
| | ATTCTCATCCCCATAGATGCAATTAGTCTGTCACTCCATT |
| | TAG |
| | [SEQ ID NO: 42] |
| | 211906_s_at: |
| | GATACGACACTGGTTCTTGTGAACGCAATCTATTTCAAAG |
| | GGCAGTGGGAGAATAAATTTAAAAAAGAAAACACTAAAGA |
| | GGAAAAATTTTGGCCAAACAAGGATGTACAGGCCAAGGTC |
| | CTGGAAATACCATACAAAGGCAAAGATCTAAGCATGATTG |
| | TGCTGCTGCCAAATGAAATCGATGGTCTGCAGAAGCTTGA |
| | AGAGAAACTCACTGCTGAGAAATTGATGGAATGGACAAGT |
| | TTGCAGAATATGAGAGAGACATGTGTCGATTTACACTTAC |
| | CTCGGTTCAAAATGGAAGAGAGCTATGACCTCAAGGACAC |
| | GTTGAGAACCATGGGAATGGTGAATATCTTCAATGGGGAT |
| | GCAGACCTCTCAGGCATGACCTGGAGCCACGGTCTCTCAG |
| | TATCTAAAGTCCTACACAAGGCCTTTGTGGAGGTCACTGA |
| | GGAGGGAGTGGAAGCTGCAGCTGCCACCGCTGTAGTAGTA |
| | GTCGAATTATCATCTCCTTCAACTAATG |
| | [SEQ ID NO: 43] |
| CST4 | Cystatin-4 |
| | 206994_at: |
| | GCGAGTACAACAAGGCCACCGAAGATGAGTACTACAGACG |
| | CCCGCTGCAGGTGCTGCGAGCCAGGGAGCAGACCTTTGGG |
| | GGGGTGAATTACTTCTTCGACGTAGAGGTGGGCCGCACCA |
| | TATGTACCAAGTCCCAGCCCAACTTGGACACCTGTGCCTT |
| | CCATGAACAGCCAGAACTGCAGAAGAAACAGTTATGCTCT |
| | TTCGAGATCTACGAAGTTCCCTGGGAGGACAGAATGTCCC |
| | TGGTGAATTCCAGGTGTCAAGAAGCCTAGGGGTCTGTGCC |
| | AGGCCAGTCACACCGACCACCACCCACTCCCACCCCCTGT |
| | AGTGCTCCCACCCCTGGACTGGTGGCCCCCACCCTGCGGG |
| | AGGCCTCCCCATGTGCCTGTGCCAAGAGACAGACAGAGAA |
| | GGCTGCAGGAGTCCTTTGTTGCTCAGCAGGGCGCTCTGCC |
| | CTCCCTCCTTCCTTCTTGCTTCTAATAGACCTGGTACATG |
| | GTACACACACCCC |
| | [SEQ ID NO: 44] |
| PRB4 | proline-rich protein BstNI subfamily |
| | 4 precursor |
| | 216881_x_at: |
| | CCACCTCCTCCAGGAAAGCCAGAAAGACCACCCCCACAAG |
| | GAGGTAACCAGTCCCAAGGTCCCCCACCTCATCCAGGAAA |
| | GCCAGAAGGACCACCCCCACAGGAAGGAAACAAGTCCCGA |
| | AGTGCCCGATCTCCTCCAGGAAAGCCACAAGGACCACCCC |
| | AACAAGAAGGCAACAAGCCTCAAGGTCCCCCACCTCCTGG |
| | AAAGCCACAAGGCCCACCCCCAGCAGGAGGCAATCCCCAG |
| | CAGCCTCAGGCACCTCCTGCTGGAAAGCCCCAGGGGCCAC |
| | CTCCACCTCCTCAAGGGGGCAGGCCACCCAGACCTGCCCA |
| | GGGACAACAGCCTCCCCAGTAATCTAGGATTCAATGACAG |
| | GAAGTGAATAAGAAGATATCAGTGAATTCAAATAATTCAA |
| | TTGCTACAAATGCCGTGACATTGGAACAAGGTCATCATAG |
| | CTCTAAC |
| | [SEQ ID NO: 45] |
| TPSD1** | 207741_x_at: |
| | TGACGCAAAATACCACCTTGGCGCCTACACGGGAGACGAC |
| | GTCCGCATCATCCGTGACGACATGCTGTGTGCCGGGAACA |
| | GCCAGAGGGACTCCTGCAAGGGCGACTCTGGAGGGCCCCT |
| | GGTGTGCAAGGTGAATGGCACCTGGCTACAGGCGGGCGTG |
| | GTCAGCTGGGACGAGGGCTGTGCCCAGCCCAACCGGCCTG |
| | GCATCTACACCCGTGTCACCTACTACTTGGACTGGATCCA |
| | CCACTATGTCCCCAAAAAGCCGTGAGTCAGGCCTGGGTGT |
| | GCCACCTGGGTCACTGGAGGACCA |
| | [SEQ ID NO: 46] |
| | Affy 216474_x_at |
| | CCGCCATTTCCTCTGAAGCAGGTGAAGGTCCCCATAATGG |
| | AAAACCACATTTGTGACGCAAAATACCACCTTGGCGCCTA |
| | CACGGGAGACGACGTCCGCATCGTCCGTGACGACATGCTG |
| | TGTGCCGGGAACACCCGGAGGGACTCATGCCAGGGCGACT |
| | CCGGAGGGCCCCTGGTGTGCAAGGTGAATGGCACCTGGCT |
| | GCAGGCGGGCGTGGTCAGCTGGGGCGAGGGCTGTGCCCAG |
| | CCCAACCGGCCTGGCATCTACACCCGTGTCACCTACTACT |
| | TGGACTGGATCCACCACTATGTCCCCAAAAAGCCGTGAGT |
| | CAGGCCTGGGTTGGCCACCTGGGTCACTGGAGGACCAA |
| | [SEQ ID NO: 47] |
| | 205683_x_at: |
| | TGACGCAAAATACCACCTTGGCGCCTACACGGGAGACGAC |
| | GTCCGCATCGTCCGTGACGACATGCTGTGTGCCGGGAACA |
| | CCCGGAGGGACTCATGCCAGGGCGACTCCGGAGGGCCCCT |
| | GGTGTGCAAGGTGAATGGCACCTGGCTGCAGGCGGGCGTG |
| | GTCAGCTGGGGCGAGGGCTGTGCCCAGCCCAACCGGCCTG |
| | GCATCTACACCCGTGTCACCTACTACTTGGACTGGATCCA |
| | CCACTATGTCCCCAAAAAGCCGTGAGTCAGGCCTGGGTTG |
| | GCCACCTGGGTCACTGGAGGACCAACCCCTGCTGTCCAAA |
| | ACACCACTGCTTCCTACCCAGGTGGCGACTGCCCCCCACA |
| | CCTTCCCTGCCCCGTCCTGAGTGCCCCTTCCTGTCCTAAG |
| | CCCCCTGCTCTCTTCTGAGCCCCTTCCCCTGTCCTGAGGA |
| | CCCTTCCCTATCCTGAGCCCCCTTCCCTGTCCTAAGCCTG |
| | ACGCCTGCACCGGGCCCTCCAGCCCTCCCCTGCCCAGATA |
| | GCTGGTGGTGGGCGCTAATCCT |
| | [SEQ ID NO: 48] |
| | 207134_x_at: |
| | TGACGCAAAATACCACCTTGGCGCCTACACGGGAGACGAC |
| | GTCCGCATCGTCCGTGACGACATGCTGTGTGCCGGGAACA |
| | CCCGGAGGGACTCATGCCAGGGCGACTCCGGAGGGCCCCT |
| | GGTGTGCAAGGTGAATGGCACCTGGCTGCAGGCGGGCGTG |
| | GTCAGCTGGGGCGAGGGCTGTGCCCAGCCCAACCGGCCTG |
| | GCATCTACACCCGTGTCACCTACTACTTGGACTGGATCCA |
| | CCACTATGTCCCCAAAAAGCCGTGAGTCAGGCCTGGGTTG |
| | GCCACCTGGGTCACTGGAGGACCAACCCCTGCTGTCCAAA |
| | ACACCACTGCTTCCTACCCAGGTGGCGACTGCCCCCCACA |
| | CCTTCCCTGCCCCGTCCTGAGTGCCCCTTCCTGTCCTAAG |
| | CCCCCTGCTCTCTTCTGAGCCCCTTCCCCTGTCCTGAGGA |
| | CCCTTCCCCATCCTGAGCCCCCTTCCCTGTCCTAAGCCTG |
| | ACGCCTGCACCGGGCCCTCCGGCCCTCCCCTGCCCAGGCA |
| | GCTGGTGGTGGGCGCT |
| | [SEQ ID NO: 49] |
| | 210084_x_at: |
| | CCGGTCAGCAGGATCATCGTGCACCCACAGTTCTACATCA |
| | TCCAGACTGGAGCGGATATCGCCCTGCTGGAGCTGGAGGA |
| | GCCCGTGAACATCTCCAGCCGCGTCCACACGGTCATGCTG |
| | CCCCCTGCCTCGGAGACCTTCCCCCCGGGGATGCCGTGCT |
| | GGGTCACTGGCTGGGGCGATGTGGACAATGATGAGCCCCT |
| | CCCACCGCCATTTCCCCTGAAGCAGGTGAAGGTCCCCATA |
| | ATGGAAAACCACATTTGTGACGCAAAATACCACCTTGGCG |
| | CCTACACGGGAGACGACGTCCGCATCATCCGTGACGACAT |
| | GCTGTGTGCCGGGAACACCCGGAGGGACTCATGCCAGGGC |
| | GACTCTGGAGGGCCCCTGGTGTGCAAGGTGAATGGCACCT |
| | GGCTACAGGCGGGCGTGGTCAGCTGGGACGAGGGCTGTGC |
| | CCAGCCCAACCGGCCTGGCATCTACACCCGTGTCACCTAC |
| | TACTTGGACTGGATCCACCACTATGTCCCCAAAAAGCCGT |
| | GAGTCAGGCCTGGGGTGT |
| | [SEQ ID NO: 50] |
| | 217023_x_at: |
| | CCGGTCAGCAGGATCATCGTGCACCCACAGTTCTACACCG |
| | CCCAGATCGGAGCGGACATCGCCCTGCTGGAGCTGGAGGA |
| | GCCGGTGAACGTCTCCAGCCACGTCCACACGGTCACCCTG |
| | CCCCCTGCCTCAGAGACCTTCCCCCCGGGGATGCCGTGCT |
| | GGGTCACTGGCTGGGGCGATGTGGACAATGATGAGCGCCT |
| | CCCACCGCCATTTCCTCTGAAGCAGGTGAAGGTCCCCATA |
| | ATGGAAAACCACATTTGTGACGCAAAATACCACCTTGGCG |
| | CCTACACGGGAGACGACGTCCGCATCGTCCGTGACGACAT |
| | GCTGTGTGCCGGGAACACCCGGAGGGACTCATGCCAGGTG |
| | GCGACT |
| | [SEQ ID NO: 51] |
| | 215382_x_at: |
| | CCGGTCAGCAGGATCATCGTGCACCCACAGTTCTACATCA |
| | TCCAGACTGGAGCGGATATCGCCCTGCTGGAGCTGGAGGA |
| | GCCCGTGAACATCTCCAGCCGCGTCCACACGGTCATGCTG |
| | CCCCCTGCCTCGGAGACCTTCCCCCCGGGNNTGCCGTGCT |
| | GGGTCACTGGCTGGGGCGATGTGGACAATGATGAGCCCCT |
| | CCCACCGCCATTTCCCCTGAAGCAGGTGAAGGTCCCCATA |
| | ATGGAAAACCACATTTGTGACGCAAAATACCACCTTGGCG |
| | CCTACACGGGAGACGACGTCCGCATCATCCGTGACGACAT |
| | GCTGTGTGCCGGGAACACCCGGAGNGNNTCATGCCAGGGC |
| | GACTCNGGAGGGCCCCTGGTGTGCAAGGTGAATGGCACCT |
| | GGCTNCAGGCGGGCGTGGTCAGCTGGGNCGAGGGCTGTGC |
| | CCAGCCCAACCGGCCTGGCATCTACACCCGTGTCACCTAC |
| | TACTTGGACTGGATCC |
| | [SEQ ID NO: 52] |
| TPSG1** | 216485_s_at: |
| | GTCGTCACGGACGATGCGGACGTCGTCTCCCGTGTAGGCG |
| | CCAAGGTGGTATTTTGCGTCACAAATGTGGTTTTCCATTA |
| | TGGGGACCTTCACCTGCTTCAGAGGAAATGGCGGTGGGAG |
| | GCGCTCATCATTGTCCACATCGCCCCAGCCAGTGACCCAG |
| | CACGGCATCCCCGGGGGGAAGGTCTCTGAGGCAGGGGGCA |
| | GGGTGACCGTGTGGACGTGGCTGGAGACGTTCACCGGCTC |
| | CTCCAGCTCCAGCAGGGCGATGTCCGCTCCGATCTGGGCG |
| | GTGTAGAACTGTGGGTGCACGATGATCCTGCTGACCGGCA |
| | GCAGCTGGTCCTGGTAGTAGAGGTGCTGCTCCCGCAGTTG |
| | CACCGGTCCCACGCAGTGCGCTGCGGTCAGCACCCACTGG |
| | GGGTGGAT |
| | [SEQ ID NO: 53] |
| | 220339_s_at: |
| | GGTGAAAGTCTCCGTGGTGGACACAGAGACCTGCCGCCGG |
| | GACTATCCCGGCCCCGGGGGCAGCATCCTTCAGCCCGACA |
| | TGCTGTGTGCCCGGGGCCCCGGGGATGCCTGCCAGGACGA |
| | CTCCGGGGGGCCTCTGGTCTGCCAGGTGAACGGTGCCTGG |
| | GTGCAGGCTGGCATTGTGAGCTGGGGTGAGGGCTGCGGCC |
| | GCCCCAACAGGCCGGGAGTCTACACTCGTGTCCCTGCCTA |
| | CGTGAACTGGATCCGCCGCCACATCACAGCATCAGGGGGC |
| | TCAGAGTCTGGGTACCCCAGGCTCCCCCTCCTGGCTGGCT |
| | TATTCCTCCCCGGCCTCTTCCTTCTGCTAGTCTCCTGTGT |
| | CCTGCTGGCCAAGTGCCTGCTGCACCCATCTGCGGATGGT |
| | ACTCCCTTCCCCGCCCCTGACTGATGGCAGGAATCCAAGT |
| | GCATTTCTTAAATAAGTTACTATTTATTCCGCTCCGCCCC |
| | CTCCCTCTCCCTTGAGAAGCTGAGTCTTCTGCATCAGATT |
| | [SEQ ID NO: 54] |
| | 213536_s_at: |
| | TGCCACAAGGTCGCTGCTTATGAGGGCGCAAACTTCTTGG |
| | CTTGTGCTCGGACCCTTTTCTCGTACTCCACTCTGTTTTG |
| | GCAGTAAATCGTGTAGGCCTCTGCTTGAGCTGGGTCTTGG |
| | ATATTTGGTTCATTTAGAAGTTCCTGTATTCCTAATAGGA |
| | TCTGTTTGATTGTGATGGCTGGCCTCCAGTCCTTGTCCTC |
| | CTCTAAGATGGACAGGCACACTGTCCCCGAAGGGTACACA |
| | TTCGGGTGAAATAATGGTGGTTCGAATTTACATTTTGGTG |
| | GCGAAGATGGATAATCATCTTTGAAAAGCATCCGTAGTTT |
| | AAACAAGCCTCCTTCCCACGGAGTCCCTTTCTTTCCTGGA |
| | ATGGCGCACTCCCAGTTCATGAGGTTCATCGTGCCATCGG |
| | GATTTTTTGTTGGGACAGCCACGAAACCAAATGGGTGGTC |
| | TTTCCTCCATGCTTTCCTCTCCTGGGCGAGTCTGCTGAGG |
| | GCGATCCCCGACATGTTCAAAGTCCCTC |
| | [SEQ ID NO: 55] |
| | 214067_at: |
| | AGAGACTTTCAGGGCATACGTGGGGGCCTTGGCCTTCCTC |
| | ACTCGCTCGATGGCCTCAGTGTGCTCCTCAAGGCTGGTGC |
| | CAAACACCTGCTGGAGATAGCTGAGCAGGGCCTCCTCGTC |
| | GTCCACCTGGTCAGGGCCCATGGTACCCGCGCGGTAAAGC |
| | ACCGTGTACAGGGCCTCCTCGTAGAGCATCTCCACCTCCT |
| | CTGGGGCCAGGGCTCTCAGGCCGAGGCTGGGATCCACAGG |
| | CTCCGGGGGTGCTGGCGAGCCACTGCGCAGGGGGACCTCG |
| | AGGCACGGCAAGCCCTGTCTGCCTTCCCCCTTCTTCAGCA |
| | TGAGGCGCATGTGGGCAAAGAACTCCACGCCATCCCCGGG |
| | TTTCCAGGCCCCCGTGGCAGGCTCCTGCGGGTCGGCGCTG |
| | GCACTCCCTGGGTCCTGCTCAGTCCTGCGGCGGAAGGACG |
| | GGCACACCTGCACCTGCCTGAGCACGCTGCTCTTAATGTC |
| | CAGCAAGGTCGACATGGCGGGTGACCGTGG |
| | [SEQ ID NO: 56] |
| MFSD2 | Major facilitator superfamily |
| | domain-containing protein 2 |
| | 225316_at: |
| | TGCTGCTCTTCAAAATGTACCCCATTGATGAGGAGAGGCG |
| | GCGGCAGAATAAGAAGGCCCTGCAGGCACTGAGGGACGAG |
| | GCCAGCAGCTCTGGCTGCTCAGAAACAGACTCCACAGAGC |
| | TGGCTAGCATCCTCTAGGGCCCGCCACGTTGCCCGAAGCC |
| | ACCATGCAGAAGGCCACAGAAGGGATCAGGACCTGTCTGC |
| | CGGCTTGCTGAGCAGCTGGACTGCAGGTGCTAGGAAGGGA |
| | ACTGAAGACTCAAGGAGGTGGCCCAGGACACTTGCTGTGC |
| | TCACTGTGGGGCCGGCTGCTCTGTGGCCTCCTGCCTCCCC |
| | TCTGCCTGCCTGTGGGGCCAAGCCCTGGGGCTGCCACTGT |
| | GAATATGCCAAGGACTGATCGGGCCTAGCCCGGAACACTA |
| | ATGTAGA |
| | [SEQ ID NO: 57] |
| CPA3** | Carboxypeptidase A3 |
| | 205624_at |
| | TATGAAACCCGCTACATCTATGGCCCAATAGAATCAACAA |
| | TTTACCCGATATCAGGTTCTTCTTTAGACTGGGCTTATGA |
| | CCTGGGCATCAAACACACATTTGCCTTTGAGCTCCGAGAT |
| | AAAGGCAAATTTGGTTTTCTCCTTCCAGAATCCCGGATAA |
| | AGCCAACGTGCAGAGAGACCATGCTAGCTGTCAAATTTAT |
| | TGCCAAGTATATCCTCAAGCATACTTCCTAAAGAACTGCC |
| | CTCTGTTTGGAATAAGCCAATTAATCCTTTTTTGTGCCTT |
| | TCATCAGAAAGTCAATCTTCAGTTATCCCCAAATGCAGCT |
| | TCTATTTCACCTGAATCCTTCTCTTGCTCATTTAAGTCCC |
| | ATGTTACTGCTGTTTGCTTTTACTTACTTTCAGTAGCACC |
| | ATAACGAAGTAGCTTTAAGTGAAACCTTTTAACTACCTTT |
| | CTTTGCTCCAAGTGAAGTTTGGACCCAGCAGAAAGCATTA |
| | TTTTGAAAGGTGATATACAGTGGGGCACAGAAAACAAATG |
| | AAAACCCTCAGTTTCTCACAGATTTTCACCATGTGGCTTC |
| | ATCAA |
| | [SEQ ID NO: 58] |
| GPR105*** | G-protein coupled receptor 105 |
| | 206637_at: |
| | TGAGCCTGGGGTTCTGGTGTTAGAATATTTTTAAGTAGGC |
| | TTTACTGAGAGAAACTAAATATTGGCATACGTTATCAGCA |
| | ACTTCCCCTGTTCAATAGTATGGGAAAAATAAGATGACTG |
| | GGAAAAAGACACACCCACACCGTAGAACATATATTAATCT |
| | ACTGGCGAATGGGAAAGGAGACCATTTTCTTAGAAAGCAA |
| | ATAAACTTGATTTTTTTAAATCTAAAATTTACATTAATGA |
| | GTGCAAAATAACACATAAAATGAAAATTCACACATCACAT |
| | TTTTCTGGAAAACAGACGGATTTTACTTCTGGAGACATGG |
| | CATACGGTTACTGACTTATGAGCTACCAAAACTAAATTCT |
| | TTCTCTGCTATTAACTGGCTAGAAGACATTCATCTATTTT |
| | TCAAATGTTCTTTCAAAACATTTTTATAAGTAATGTTTGT |
| | ATCTATTTCATGCTTTACT |
| | [SEQ ID NO: 59] |
| CDH26 | Cadherin-like protein 26 [Precursor] |
| | 232306_at: |
| | GGGAATCACTATTCAGGGATTTTTCCCCTTTGCTCTTCTT |
| | TTCCCTCCTTAAAAGAAAAATTACCTTCTAGTCCTAGGAT |
| | GAGGACACACTATTAGTTTGAATTAAATGCTTTGATATTC |
| | TCAGATCAGCCATCTTGAACCAAAGCAAAACCACAAGTTA |
| | CACTTTCTTAAAATTTGATTTGTCATATTTTCTAGAGAAA |
| | CTTGAATTTAATTGTGTTATTCTTAGCTTCCACTGGCAGC |
| | CTAGCTTTGAGGGTAAATGAAAATATAACCCATAGATTAC |
| | CCAGCCACTTGGGAACAGCAGGTAATACTGAAGAAAAATA |
| | AAAATAGATTTTGAAAACGTTANNNANANNNNTATGATTA |
| | TGATTCTGTTCCATTTAAGGGAAAACTTAGGTAAATAGAG |
| | AAATTTTTTCTATAACATTGTGTAGTCAGT |
| | [SEQ ID NO: 60] |
| GSN | Gelsolin [Precursor] |
| | 200696_s_at: |
| | TGCTTCTGGACACCTGGGACCAGGTCTTTGTCTGGGTTGG |
| | AAAGGATTCTCAAGAAGAAGAAAAGACAGAAGCCTTGACT |
| | TCTGCTAAGCGGTACATCGAGACGGACCCAGCCAATCGGG |
| | ATCGGCGGACGCCCATCACCGTGGTGAAGCAAGGCTTTGA |
| | GCCTCCCTCCTTTGTGGGCTGGTTCCTTGGCTGGGATGAT |
| | GATTACTGGTCTGTGGACCCCTTGGACAGGGCCATGGCTG |
| | AGCTGGCTGCCTGAGGAGGGGCAGGGCCCACCCATGTCAC |
| | CGGTCAGTGCCTTTTGGAACTGTCCTTCCCTCAAAGAGGC |
| | CTTAGAGCGAGCAGAGCAGCTCTGCTATGAGTGTGTGT |
| | [SEQ ID NO: 61] |
| C2ORF32 | 226751_at: |
| | ACTTTTGACCACTTGTGACTGGAGTTCAGTGGCCCTGGCA |
| | GGCTTGTCCTGCTCTTGACCATTCCACTGACTAACTTTGG |
| | TGTTTNGTTTCCAAGTTAAGTGATTCCTCCTTTTTTTNGT |
| | TCAATGTTAAATTTAAAAATAACAATGTGTATGGGTCCTC |
| | CCATGTGTAATATGGTAACATGTAACTTGCAGTGTTTGCC |
| | AGCTTTCAAAGCAGGCTTTGTGAAAATGTAATACAAACAG |
| | CAGTGAATGGGACTCAAATGTTGTGCTTCCTATAAACAGC |
| | TCCGCTCTTTCAGGGAAGGATGGTAACAAACTAGAAGGAC |
| | AAATATGTACGTATTTATAACGTATTAAAACTCTTTTAAG |
| | TAGCTTAAGGTATTGTGCAATGGCCTAGCCTAGTAGAAAT |
| | GGGGGAAAAGCATTGCTGTGGACCATTGTTAAAGTGACAG |
| | GAGTTGTAGGGTTACCCCTTTGACAAGCTTCCATAGTCTT |
| | CAGACACGCACATTGATGGCATCCCT |
| | [SEQ ID NO: 62] |
| TRACH | 238429_at: |
| 2000196 | CTAACTAATACCAACCTGACAACTTGAATAACAATAAATG |
| (TMEM71) | CAATTTGTACATAAAATATNATGCTGCAAAAGTTNGTCAT |
| | TCACCTCAGTGGAGTGACTTGATATTAGGTGGTNACCGTA |
| | GATGATGGTTNATATGANAANTGGACAGGAAAGAAGCANT |
| | TTCTGAAAGTTATANTCTTTTGAACCACGTTCTAAACCAA |
| | GTNTTTNATCTTCTTGGGGCTCGTAATTACCTTTCACTTT |
| | AATGTCACTTAAAGATATAACACAGAAAAATGCCTTGAGG |
| | GCAAAATATAGGCAAAACACCAATGCGCTTTCAAATGCAT |
| | GAAAATGGTGCAGTTGTACCCTTGAGCCTTGACTCAAGGG |
| | CTGTAGATGTTCCCTTTCCACCCCCCACACTTGGTGCGTG |
| | TTCACAAAGCAAATATGGCCTGTAATTCAAATTTGTTCTA |
| | TGTGATACTCTCTGAGTAAAAACTCATACATGCAGAAAAT |
| | TGTCTTTGCTCGAAAT |
| | [SEQ ID NO: 63] |
| DNAJC12 | DnaJ homolog subfamily C member 12 |
| | 218976_at: |
| | CCCAAGCCCCTAGAGAAGTCAGTCTCCCCGCAAAATTCAG |
| | ATTCTTCAGGTTTTGCAGATGTGAATGGTTGGCACCTTCG |
| | TTTCCGCTGGTCCAAGGATGCTCCCTCAGAACTCCTGAGG |
| | AAGTTCAGAAACTATGAAATATGAAATATCTCTGCTTCAA |
| | AAAATGAGGAAGAGCAAGACTGTCCCCTATGCTGCCAACA |
| | TGCAGTCTTTGTTTATGTCTTAAAAATGTCATGTTTATGT |
| | CATGTCTGTGAATTGCTGAGTACTAATTGATTCCTCCATC |
| | CTTGAATCAGTTCTCATAATGCTTTTTAAATAAGAAAAAT |
| | TCAGAAGATGAATTTCTTCCAATATTTGAATAAATTAAAG |
| | CTCTTAGATACAGAGTAGATTGTATTATATGCTTTTTCCT |
| | ATTAATACTACTTATAGAAATCCATTAAAAAGCAATCTCT |
| | GTACAGTGTATTTAAATATTTCATTGACATACTGTGATCT |
| | CTATTAGTGATGGATGTACAAAAAATGTTTTCTTACCCTT |
| | GACTTACAATGAAATGTGAAATTACTTGTCTGAACCCCGT |
| | [SEQ ID NO: 64] |
| RGS13** | Regulator of G-protein signaling 13 |
| | 210258_at: |
| | ACAGCAAGCCTATGTAGTTCAATTAATATATAAGGAAAAG |
| | GAAGGTCTTTCTTCATGATACAAGCATTATAAAGTTTTTA |
| | CTGTAGTAGTCAATTAATGGATATTTCCTTGTTAATAAAA |
| | TTTTGTGTCATAATTTACAAATTAGTTCTTTAAAAATTGT |
| | TGTTATATGAATTGTGTTTCTAGCATGAATGTTCTATAGA |
| | GTACTCTAAATAACTTGAATTTATAGACAAATGCTACTCA |
| | CAGTACAATCAATTGTATTATACCATGAGAAAATCAAAAA |
| | GGTGTTCTTCAGAGACATTTTATCTATAAAATTTTCCTAC |
| | TATTATGTTCATTAACAAACTTCTTTATCACATGTATCTT |
| | CTACGTGTAAAACATTTCTGATGATTTTTTAACAAAAAAT |
| | ATATGAATTTCTTCATTTGCTCTTGCATCTACATTGCTAT |
| | AANGGATATAAAATGTGGTTTCTATATTTTGAGATGTTTT |
| | TTCCTTACAATGTGAACTCATCGTGATCTTGG |
| | [SEQ ID NO: 65] |
| SLC18A2** | Solute carrier family 18 member 2 |
| | 205857_at: |
| | CTGCTACTTTGGAAGATGGCTCTGGAGGAAACTCTCATAT |
| | GGCTAAAAAGGCAGGCTAGTTTCTTACTTCTACAGGGGTA |
| | GAGCCTTAAAAAAGAACGTGCTACAAATTGGTTNTCTTNN |
| | AGGGTTNCNGGTTCTCCCTGCCCCCAATNCCNATATACTT |
| | TANTGCNNTTTTATTTTTGCCTTTACGGNCTCTGTGTCTT |
| | TCTGCAAGAAGGCCTGGCAAAGGTATGCCTGCTGTTGGTC |
| | CCNTCGGGATAAGATAAAATATAAATAAAACCTTCAGAAC |
| | TGTTTTGGAGCAAAAGATAGCTTGTACTTGGGGAAAAAAA |
| | TTCTAAGTTCTTTTATATGACTAATATTCTTGGTTAGCAA |
| | GACTGGAAAGAGGTGTTTTTTTAAAATGTACATACCAGAA |
| | CAAAGAACATACAGCTCTCTGAACATTTATTTTTTGAACA |
| | GAGGTGGTTTTTATGTTTGGACCTGGTAATACAGATACAA |
| | AAACTTTAATGAGGTAGCAATGAATATTCAACTGTTTGAC |
| | TGCTAAGTGTATCTGTCCATATTTTAGCAAG |
| | [SEQ ID NO: 66] |
| SERPINB10 | Serpin peptidase inhibitor clade B |
| | (Ovalbumin) member 10 |
| | 214539_at: |
| | TACTACAAAAGCCGTGACCTCAGCCTGCTTATACTACTGC |
| | CAGAAGACATTAATGGGCTGGAACAGCTGGAAAAGGCCAT |
| | CACCTATGAGAAGCTGAATGAGTGGACCAGTGCAGACATG |
| | ATGGAGTTGTATGAAGTGCAGCTACACCTTCCCAAGTTCA |
| | AGCTGGAAGACAGTTATGATCTCAAGTCAACCCTGAGCAG |
| | TATGGGGATGAGTGATGCCTTCAGCCAAAGCAAAGCTGAT |
| | TTCTCAGGAATGTCTTCAGCAAGAAACCTATTTTTGTCCA |
| | ATGTTTTCCATAAGGCTTTTGTGGAAATAAATGAACAAGG |
| | TACTGAAGCTGCAGCTGGCAGTGGGAGTGAGATAGATATA |
| | CGAANTAGAGTCCCATCCATTGAATTCAATGCAAATCACC |
| | CATTCCTCTTCTTCATCAGGCACAATAANAACCAACACCA |
| | TTCTTTTTTATGGAAGATTATGCTCCCCCTAATC |
| | [SEQ ID NO: 67] |
| SH3RF2 | SH3 domain-containing RING finger |
| | protein 2 |
| | 243582_at: |
| | GATTCTGTGGTAGACTCAGTGCTTTCAGAGTCCAGAGCTT |
| | GACTTGGGTTAGTGGCCTTAATGAAGTGCTAAATTTGCTC |
| | TTTACCGCGAGACTGATCAGAAGAAGCAAAAGGGGAAAGG |
| | GGGCTAGAGGTCCACTCGCACCTTTTACATCAGACAAGAG |
| | GAGGACTGTGCCAGAAATCTGTGCATGAAACACCATCTGC |
| | TCTTCATGCAGGGAGGGGTCAACCGTGTGAACGTGCAGAG |
| | ATTACTCGAGCCTTCTTTGCCAAAAATATGCATTCTTCCC |
| | AGCTGTA |
| | [SEQ ID NO: 68] |
| FCER1B** | FcepsilonRIbeta |
| | 207496_at: |
| | TAATCACATCACTTCCATGGCATGGATGTTCACATACAGA |
| | CTCTTAACCCTGGTTTACCAGGACCTCTAGGAGTGGATCC |
| | AATCTATATCTTTACAGTTGTATAGTATATGATATCTCTT |
| | TTATTTCACTCAATTTATATTTTCATCATTGACTACATAT |
| | TTCTTATACACAACACACAATTTATGAATTTTTTCTCAAG |
| | ATCATTCTGAGAGTTGCCCCACCCTACCTGCCTTTTATAG |
| | TACGCCCACCTCAGGCAGACACAGAGCACAATGCTGGGGT |
| | TCTCTTCACACTATCACTGCCCCAAATTGTCTTTCTAAAT |
| | TTCAACTTCAATGTCATCTTCTCCATGAAGACCACTGAAT |
| | GAACACCTTTTCATCCAGCCTTAATTTCTTGCTCCATAAC |
| | TACTCTATCCCACGATGCAGTATTGTATCATTAATTATTA |
| | GTGTGCTTGTGACCTCCTTATGTATTCTCAATTACCTGTA |
| | TTTGTGCAATAAATTGGAATAATGTAACTTGATTTCTTAT |
| | CTGTGTTTGTGTTGGCATGCAAGAT |
| | [SEQ ID NO: 69] |
| RUNX2 | Runt-related transcription factor 2 |
| | 232231_at: |
| | AAGACACTTCTTCCAAACCTTGAATTTGTTGTTTTTAGAA |
| | AACGAATGCATTTAAAAATATTTTCTATGTGAGAATTTTT |
| | TAGATGTGTGTTTACTTCATGTTTACAAATAACTGTTTGC |
| | TTTTTAATGCAGTACTTTGAAATATATCAGCCAAAACCAT |
| | AACTTACAATAATTTCTTAGGTATTCTGAATAAAATTCCA |
| | TTTCTTTTGGATATGCTTTACCATTCTTAGGTTTCTGTGG |
| | AACAAAAATATTTGTAGCATTTTGTGTAAATACAAGCTTT |
| | CATTTTTATTTTTTCCAATTGCTATTGCCCAAGAATTGCT |
| | TTCCATGCACATATTGTAAAAATTCCGCTTTGTGCCACAG |
| | GTCATGATTGTGGATGAGTTTACTCTTAACTTCAAAGGGA |
| | CTATTTGTATTGTATGTTGC |
| | [SEQ ID NO: 70] |
| PTGS1 | Prostaglandin-endoperoxide |

| | |
|---|---|
| | synthase 1 |
| | 238669_at: |
| | AGTATTGACAACTGCACATGAAAGTTTTGCAAAGGGAAAC |
| | AGGCTAAATGCACCAAGAAAGCTTCTTCAGAGTGAAGAAT |
| | CTTAATGCTTGTAATTTAAACATTTGTTCCTGGAGTTTTG |
| | ATTTGGTGGATGTGATGGTTGGTTTTATTTGTCAGTTTGG |
| | TTGGGCTATAGCACACAGTTATTTAATCAAACAGTAATCT |
| | AGGTGTGGCTGTGAAGGTATTTTGTAGATGTGATTAACAT |
| | CTACAATCAGTTGACTTTAAGTGAAAGAGATTACTTAAAT |
| | AATTTGGGTGAGCTGCACCTGATTAGTTGAAAGGCCTCAA |
| | GAACAAACACTGCAGTTTCCTGGAAAAGAAGAAACTTTGC |
| | CTCAAGACTATAGCCATCGACTCCTGCCTGAGTTTCCAGC |
| | CTGCTAGTCTGCCCTATGGATTTGAAGTTTGCCAACCCCA |
| | ACAATTGTGTGAATTAATTTCTAAAAATAAAGCTATATAC |
| | AGCCANNNNNNNNTATTTGTGGGGGATTTGTTTCAGGATC |
| | TCTACAGATACCAA |
| | [SEQ ID NO: 71] |
| ALOX15*** | Arachidonate 15-lipoxygenase |
| | 207328_at: |
| | CCCTAGAGGGGCACCTTTTCATGGTCTCTGCACCCAGTGA |
| | ACACATTTTACTCTAGAGGCATCACCTGGGACCTTACTCC |
| | TCTTTCCTTCCTTCCTCCTTTCCTATCTTCCTTCCTCTCT |
| | CTCTTCCTCTTTCTTCATTCAGATCTATATGGCAAATAGC |
| | CACAATTATATAAATCATTTCAAGACTAGAATAGGGGGAT |
| | ATAATACATATTACTCCACACCTTTTATGAATCAAATATG |
| | ATTTTTTTGTTGTTGTTAAGACAGAGTCTCACTTTGACAC |
| | CCAGGCTGGAGTGCAGTGGTGCCATCACCACGGCTCACTG |
| | CAGCCTCAGCGTCCTGGGCTCAAATGATCCTCCCACCTCA |
| | GCCTCCTGAGTAGCTGGGACTACAGGCTCATGCCATCATG |
| | CCCAGCTAATATTTTTTTATTTTCGTGGAGACGGGGCCTC |
| | ACTATGTTGCCTAGGCTGGAAATAGGATTTTGAACCCA |
| | [SEQ ID NO: 72] |

**Mast cell-specific genes
***Eosinophil-specific genes

### Example 8

### Relationship of “IL-13 High” and “IL-13 Low” Subphenotypes of Asthma to Clinical Features

The asthmatic subjects were further analyzed with respect to additional demographic characteristics and clinical features as those described in Example 7. The results are shown in Table 5 and FIGS. 5 and 6. Although subjects with “IL-13 high” asthma subphenotype could not be distinguished from subjects with “IL-13 low” asthma subphenotype based on demographic characteristics, lung function, or bronchodilator responsiveness (delta FEV1 with albuterol) (Table 5, FIGS. 5A-B), these groups differed significantly with respect to degree of airway hyper-responsiveness (AHR, PC₂₀ to methacholine, defined as the minimal concentration of methacholine required to induce a 20% decrease in expiratory airflow, FIG. 5C). This difference in AHR was apparent despite inclusion criteria that required all asthmatics to have significant AHR (all asthmatics <8 mg/ml, all healthy controls >20 mg/ml).

TABLE 5
Subject characteristics by asthma phenotype

| | | | Asthma | | | | |
|---|---|---|---|---|---|---|---|
| | | | IL13 | | IL-13 | | p-value |
| | Healthy | | Signature | | Signature | | low vs. |
| | Control | | Low | | High | | high |
| Sample size | 28 | | 20 | | 22 | | — |
| Age | 36 ± 9 | | 36 ± 11 | | 37 ± 12 | | 0.98 |
| Gender, M:F (% F) | 12:16 | (56) | 6:14 | (70) | 11:11 | (50) | 0.19 |
| Ethnicity | | | | | | | |
| Caucasian | 20 | | 9 | | 9 | | 0.98 |
| African-American | 0 | | 4 | | 4 | | |
| Hispanic | 3 | | 5 | | 6 | | |
| Asian/Pacific Islander | 5 | | 2 | | 3 | | |
| FEV₁, % predicted | 107 | (13) | 89 | (10) | 85 | (13) | 0.85 |
| ΔFEV₁ with albuterol | 2.7 ± 3.4% | | 9.7 ± 7.4% | | 12.5 ± 9.8 | | 0.51 |
| (% of baseline) | | | | | | | |
| Methacholine PC₂₀ | 64 | (22-64) | 0.93 | (0.06-7.3) | 0.27 | (0.05-1.9) | <0.001 |
| IgE, IU/ml | 27 | (3-287) | 125 | (19-1194) | 244 | (32-2627) | 0.031 |
| | N = 26 | | | | | | |
| Blood | 0.10 ± 0.07 | | 0.23 ± 0.21 | | 0.37 ± 0.22 | | 0.027 |
| eosinophils, ×10⁹/L | | | | | | | |

| BAL eosinophil % | 0.26 ± 0.29 | 0.42 ± 0.46 | 1.9 ± 1.9 | 0.001 |
|---|---|---|---|---|
| | N = 22 | N = 16 | N = 20 | |
| RBM thickness, μm | 4.34 ± 1.11 | 4.67 ± 0.99 | 5.91 ± 1.72 | 0.014 |
| | N = 22 | N = 19 | N = 19 | |
| ΔFEV₁ with fluticasone | N/A | 0.03 ± 0.12 | 0.35 ± 0.2 | 0.004 |
| at 4 weeks, L | | N = 6 | N = 10 | |
| ΔFEV₁ with fluticasone | N/A | 0.04 ± 0.12 | 0.25 ± 0.23 | 0.05 |
| 8 weeks, L | | N = 6 | N = 10 | |

For normally distributed data, values are presented as mean ± standard deviation and student's t-test performed; for non-normally distributed data, values are presented as median (range) and wilcoxon rank sum test performed. In case of missing data, number of subjects for whom data exist noted. P-values relative to healthy control also depicted in FIGS. 5 and 6. PC₂₀ denotes the provocative concentration required to cause a 20% decline in FEV₁; BAL, bronchoalveolar lavage; RBM, reticular basement membrane.

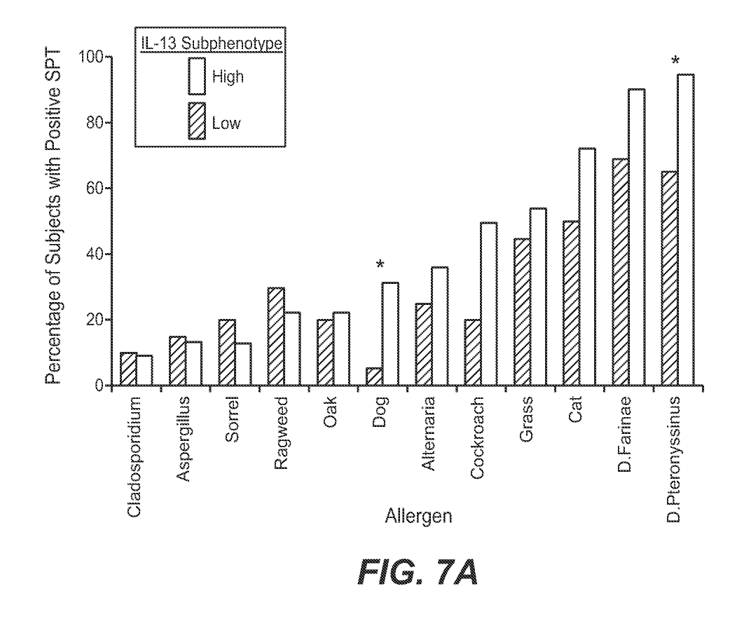

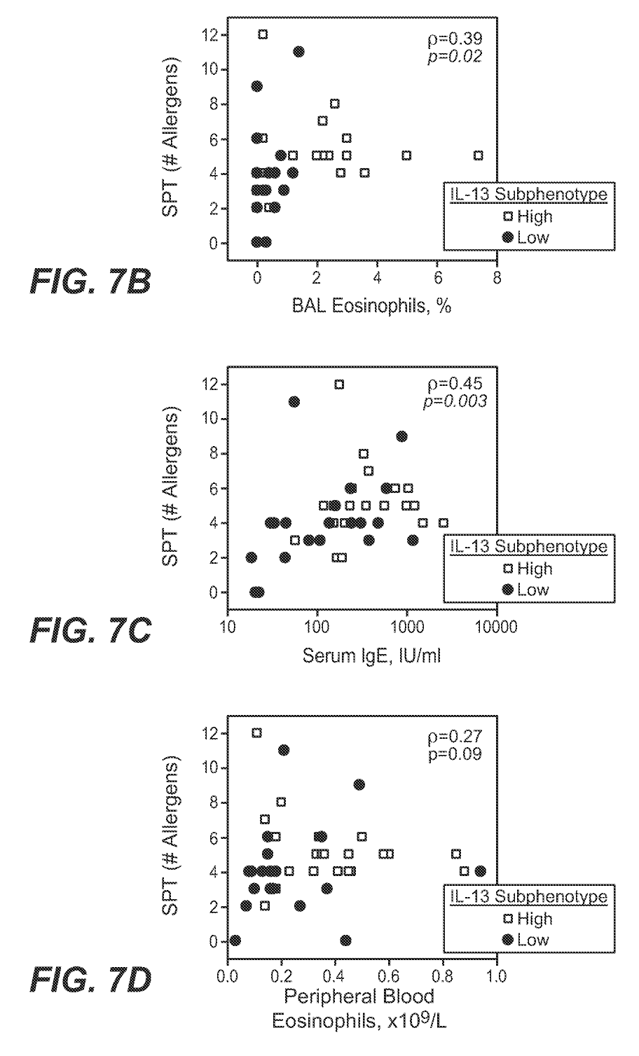

To determine whether the IL-13 subphenotype of an individual subject was correlated with measures of allergic inflammation, we examined the results of skin prick tests (SPT) to a panel of 12 aeroallergens (Table 6), levels of serum IgE, peripheral blood eosinophil counts, and eosinophil percentages in bronchoalveolar lavage fluid (BAL). The results are shown in FIGS. 6A-D and 7A-B. Both IL-13 high and low asthma subphenotypes had increased SPT sensitivity to aeroallergens as compared to healthy controls (FIG. 6A), although the IL-13 low asthma subphenotype tended to have fewer positive skin tests than the IL-13 high asthma subphenotype and to be sensitized less frequently to aeroallergens such as dog and house dust mite (FIG. 7A). Subjects with IL-13 high asthma subphenotype had higher serum IgE levels and higher peripheral blood eosinophil counts than subjects with IL-13 low asthma subphenotype, although IL-13 low asthma subphenotype differed from healthy controls with respect to these features of allergic inflammation (FIGS. 6B-C). In addition, subjects with IL-13 high asthma subphenotype had increased eosinophil numbers in the lung as assessed by BAL (FIG. 6D), whereas IL-13 low asthmatics did not differ from healthy controls in BAL eosinophil percentage. These data demonstrate enrichment for AHR, IgE levels, and eosinophilic inflammation in subjects with the IL-13 high asthma subphenotype, but SPT sensitivity to aeroallergens was not restricted to this subgroup. Thus, it is likely that alternate non-Th2 mechanisms for sensitization to aeroallergens operate in subjects with the IL-13 low asthma subphenotype.

TABLE 6
Allergen skin prick test panel
Allergen

| | | |
|---|---|---|
| D. farinae | Cladosporium herbarum | West Oak mix |
| D. pteronyssius | Cat | Grass mix/Bermuda/ |
| | | Johnson |
| American | Dog | Histamine [10 mg/ml] |
| Cockroach | | (positive control) |
| Alternaria tenuis | Plantain-Sorrel mix | 50% Glycerin |
| | | (negative control) |
| Aspergillus mix | Short Ragweed | |

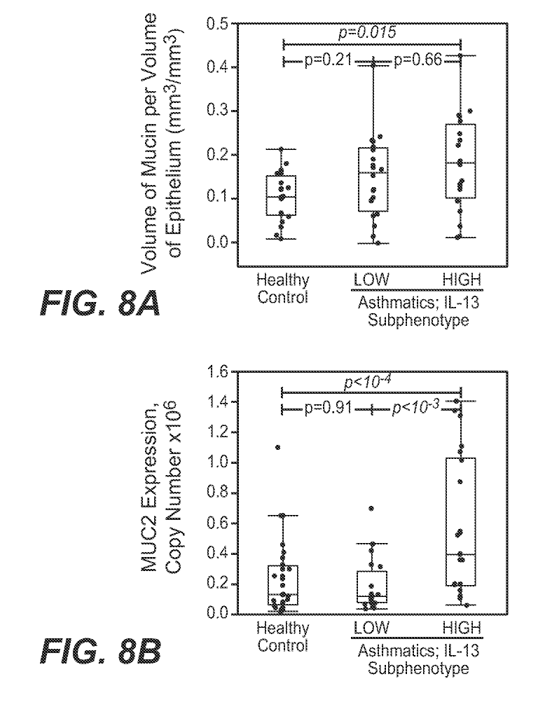

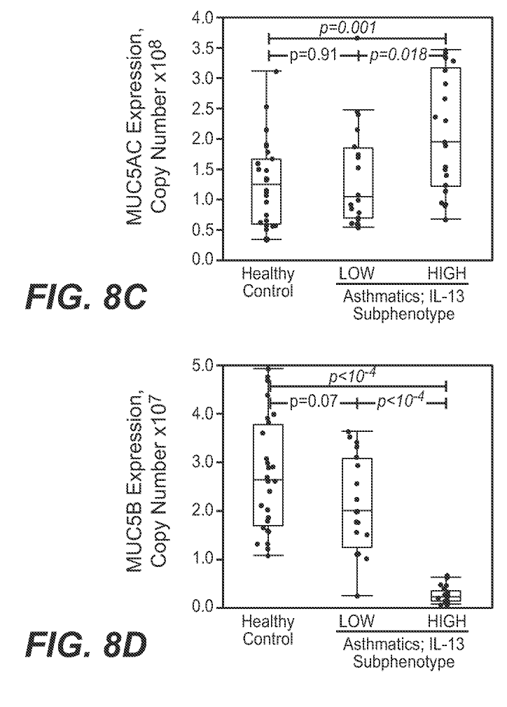

To determine whether the subphenotype of IL-13 high asthma is durable or a transient manifestation of Th2-driven inflammation due to recent exposure to allergen, we measured pathological changes in bronchial biopsies from the same subjects. We and others have previously demonstrated that asthma is associated with pathological changes known as airway remodeling and which reflect either longstanding inflammation or the effects of injury and repair over time [28, 29]. Two specific remodeling outcomes in asthma are airway fibrosis, manifest as thickening of the sub-epithelial reticular basement membrane (RBM) [30, 31] and increased mucin stores in the airway epithelium [32]. We found that RBM thickness was greater in subjects with IL-13 high asthma subphenotype than in IL-13 low asthma subphenotype or healthy controls and that RBM thickness was normal in the IL-13 low subphenotype of asthma (FIG. 6E). In addition, although we observed a trend toward increased epithelial mucin stores in both subphenotypes of subjects with asthma, this increase was significant only in subjects with IL-13 high asthma subphenotype (FIG. 8A). Although these differences in total mucin stores were modest, qPCR revealed a striking difference in the expression levels of the major gel-forming mucins in airway epithelial cells in IL-13 high asthma subphenotype as compared to both IL-13 low asthma subphenotype and healthy controls (FIGS. 8B-D). Specifically, IL-13 high asthma subphenotype was distinguished from IL-13 low asthma subphenotype and healthy controls by induction of MUC5AC and MUC2 expression and repression of MUC5B expression. This alteration in the expression of specific mucin genes in IL-13 high asthma subphenotype is most evident in the ratio of MUC5AC to MUC5B expression (FIG. 6F). Without being bound by theory, we speculate that concomitant induction and repression of specific gel-forming mucins may explain the relatively modest increase in epithelial mucin stores in IL-13 high asthma subphenotype compared to IL-13 low asthma subphenotype and healthy controls. Taken together, these findings indicate that IL-13 high asthma subphenotype is associated with remodeling changes in the airway that identify this subphenotype as durable over time. These results also demonstrate the importance of the IL-13 pathway to airway remodeling in human subjects.

Alveolar macrophages may modulate allergic airway inflammation in asthma as a source of IL-13 [54] and leukotrienes or eicosanoid lipids [55, 56] or through “alternative activation” under the influence of IL-13 [57]. To determine whether alveolar macrophages from subjects with “IL-13 high” asthma manifest any of these findings, we measured the expression of relevant genes using qPCR in 14 subjects with asthma and 15 healthy controls (Table 7). We found no evidence for induction of Th2 cytokines or of alternative activation markers in asthma generally or in the “IL-13 high” subgroup specifically. Levels of expression of IL-13 were below the limit of detection (cycle threshold >40) in 26 of the 29 subjects, and IL-4 was below the limit of detection in 20 of the 29 subjects (no differences between the three groups for either cytokine, all p>0.35). All other genes were within the limit of detection across samples. In these analyses we found increased expression of 15-lipoxygenase in “IL-13 high” asthma (FIG. 10, Table 8), consistent with prior findings of increased 15-lipoxygenase products in the airways in severe eosinophilic asthma [56]. We also found an increase in expression of TNFα that was limited to the “IL-13 high” subgroup (FIG. 10, Table 8).

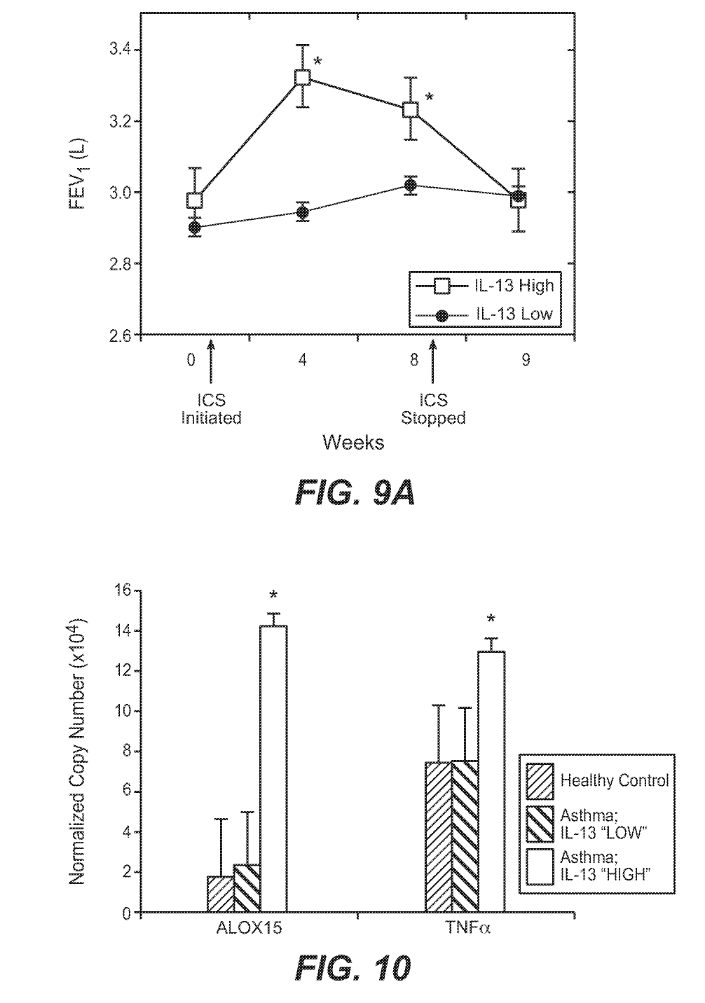

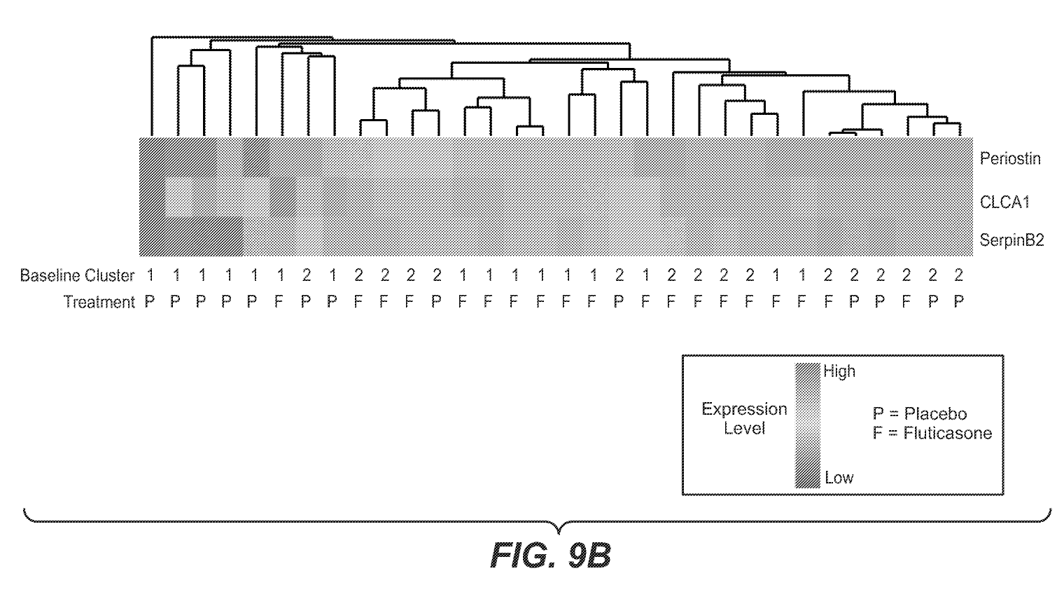

Only a subset of asthmatics manifests improvement in lung function when treated with inhaled corticosteroids (ICS) [33]. To identify gene expression markers of corticosteroid responsiveness, we measured FEV₁ in a subset of our subjects with asthma during an 8-week randomized controlled trial of inhaled fluticasone or placebo as previously reported[8]. When we re-analyzed that data while stratifying subjects by IL-13 subphenotype, we found that improvements in FEV₁ were limited to those with the IL-13 high subphenotype. Specifically, the subjects with the IL-13 high asthma subphenotype who were treated with inhaled fluticasone had significant improvements in FEV₁ at both 4 and 8 weeks as compared to subjects treated with placebo, whereas subjects with IL-13 low asthma subphenotype did not (FIG. 9A). These improvements in FEV₁ in the IL-13 high group were lost after a one week run out period off drug. There was no significant change in FEV₁ in response to placebo at any timepoint in either group (data not shown, N=5 “IL-13 high,” N=6 “IL-13 low”). As described previously [8], we performed a second bronchoscopy one week after the initiation of treatment and analyzed gene expression in bronchial epithelium by microarray as at baseline. In re-analyses of these data, while stratifying subjects by IL-13 subphenotype, subjects with IL-13 high asthma at baseline continued to exhibit a strong IL-13 subphenotype after one week of placebo treatment demonstrating the short-term stability of this subphenotype in the absence of therapy. However, after one week of fluticasone treatment, subjects with IL-13 high asthma clustered with subjects who were IL-13 low at baseline, regardless of treatment (FIG. 9B). Thus, the phenotypic classification of asthma based on the IL-13 signature described herein predicts response to ICS. These data suggest that the global benefit of ICS treatment for asthma is accounted for by the IL-13 high subphenotype.

Our results provide new insights into molecular mechanisms that underlie clinical heterogeneity in asthma. Basic research previously established IL-13 and related Th2 cytokines as central regulators of allergic inflammation and many of the pathophysiologic changes associated with asthma [35, 36]. Here, using gene expression profiling, we have identified an “IL-13 high” subphenotype in patients with asthma. Using rigorous clinical criteria and methacholine challenge testing, we found that that this subphenotype comprises only ˜50% of patients who are diagnosed with asthma. This “IL-13 high” subphenotype also displayed increased levels of IL-5 expression and showed certain distinguishing clinical characteristics including enhanced airway hyper-responsiveness, increased serum IgE levels and eosinophilic inflammation, subepithelial fibrosis, and altered expression of gel-forming mucins compared to an “IL-13 low” subphenotype and healthy controls.

Our work challenges certain current concepts of asthma pathogenesis by showing that a gene signature for IL-13 driven inflammation in airway epithelial cells is prominent in only half of asthmatics; non-IL-13 driven mechanisms must therefore operate in the remaining half. The findings discussed herein lead us to propose that asthma can be divided into various molecular subphenotypes such as “IL-13 high” and “IL-13 low” subphenotypes referred to herein. We validated the IL-13 high/IL-13 low classification scheme through confirmatory analyses of gene expression in bronchial biopsies, analysis of reproducibility on repeat examination, and comprehensive characterization of the distinct clinical, inflammatory, pathological and treatment-related characteristics of these two molecular subphenotypes of asthma. These findings provide a mechanistic framework for the emerging clinical observation that asthma is a complex and heterogeneous disease [58].

Molecular phenotyping of asthma based on Th2 inflammation has important therapeutic implications. First, airway obstruction in the “IL-13 high” subphenotype improves with inhaled steroids whereas the “IL-13 low” subphenotype shows little to no improvement. The Th2 markers that we have identified can be used to guide the development of clinical tests for steroid-responsiveness by providing surrogate markers of a steroid-responsive phenotype. Second, blockade of IL-13 and related Th2 cytokines is under active clinical development as a therapeutic strategy in asthma [34]. Our data suggest that clinical response to these therapies may be limited to the specific subphenotype of patients with “IL-13 high” asthma. Thus, markers of this molecular phenotype have direct application in clinical trials.

Prior studies using induced sputum analyses suggested that “eosinophilic asthma” is a distinct cellular phenotype of asthma, but molecular mechanisms underlying this cellular phenotype have been undefined. Our data suggest that IL-13 driven inflammation is a molecular mechanism underlying “eosinophilic asthma” [37] because of the airway eosinophilia that we demonstrated in “IL-13 high asthma.” In addition, we demonstrated that both “eosinophilic asthma” and “IL-13 high” asthma are characterized by subepithelial fibrosis [38, 39], ALOX15 production by alveolar macrophages [55] and lung function responses to inhaled corticosteroids [40, 41]. In addition to these recognized features of eosinophilic asthma, we have identified further clinical features of “IL-13 high” asthma, including altered airway mucin gene expression and induction of TNFα, a mediator which is not considered a Th2-cytokine but which has been previously associated with severe asthma [59]. We speculate that these features will also be found in eosinophilic asthma. In addition, it is likely that IL-5 is a major contributor to the airway and systemic eosinophilia we observe in “IL-13 high” asthma, because we found that IL-5 expression is significantly co-regulated with IL-13 expression (FIG. 1E). IL-5 is a major stimulus of eosinophil differentiation, recruitment, activation, and survival [60], but IL-13 can strongly induce the expression of eosinophil chemoattractants such as CCL11, CCL22, and CCL26 in the airway [61] and may thus work cooperatively with IL-5 to promote eosinophil infiltration, activation, and survival in the airways. Residual IL-13 activity may therefore explain the incomplete tissue depletion of eosinophils observed in clinical trials of IL-5 blockade in asthma [62, 63].

In addition, these data reveal that a significant percentage of patients with asthma have an “IL-13 low” phenotype which manifests such clinical features of asthma as airway obstruction, airway hyper-responsiveness and bronchodilator reversibility despite a paucity of Th2-driven inflammation. The causes of “IL-13 low” asthma remain obscure, but possibilities include neutrophilic inflammation [37], IL-17 driven inflammation [42], intrinsic defects in barrier function [43] and chronic sub-clinical infection by atypical intracellular bacteria [44].

TABLE 7
Genes used in alveolar macrophage qPCR

| Symbol | Name | Category | Entrez Gene ID |
|---|---|---|---|
| IL13 | interleukin 13 | Th2 cytokine | 3596 |
| IL4 | interleukin 4 | Th2 cytokine | 3565 |
| ARG1 | arginase, liver | Alternative activation marker | 383 |
| MRC1 | mannose receptor, C type1 | Alternative activation marker | 4360 |
| MRC2 | mannose receptor, C type2 | Alternative activation marker | 9902 |
| IL1RN | interleukin 1 receptor antagonist | Alternative activation marker | 3557 |
| CCL17 | T cell-directed CC chemokine | Alternative activation marker | 6361 |
| CCL22 | macrophage derived chemokine | Alternative activation marker | 6367 |
| TNFα | tumor necrosis factor | Classical activation marker | 7124 |
| IL1β | interleukin 1, beta | Classical activation marker | 3553 |
| CCL20 | macrophage inflammatory | Classical activation marker | 6364 |
| | protein 3 alpha | | |
| ALOX15 | arachidonate 15-lipoxygenase | Leukotriene pathway | 246 |
| ALOX5 | arachidonate 5-lipoxygenase | Leukotriene pathway | 240 |
| ALOX5AP | arachidonate 5-lipoxygenase- | Leukotriene pathway | 241 |
| | activating protein | | |
| LTA4H | leukotriene A4 hydrolase | Leukotriene pathway | 4048 |
| LTC4S | leukotriene C4 synthase | Leukotriene pathway | 4056 |

TABLE 8
Alveolar macrophage gene expression by qPCR

| | Normalized Gene Copy Number | | | P-values | | |
|---|---|---|---|---|---|---|
| | Control | IL-13 Low | IL-13 High | IL-13 Low | IL-13 High | IL-13 High |
| Gene | N = 15 | N = 5 | N = 9 | vs. control | vs. control | vs IL-13 Low |
| IL13 | — | — | — | — | — | — |
| IL4 | — | — | — | — | — | — |
| ARG1 | 16,707 ± 49,889 | 13,188 ± 29,285 | 177 ± 349 | 0.99 | 0.68 | 0.91 |
| MRC1 | 4,729,405 ± 2,343,659 | 4,281,358 ± 2,235,805 | 5,575,399 ± 2,211,337 | 0.98 | 0.77 | 0.69 |
| MRC2 | 323,199 ± 949,034 | 318,115 ± 704,525 | 1,627 ± 929 | 1.00 | 0.68 | 0.84 |
| IL1RN | 1,217,545 ± 2,179,904 | 1,629,394 ± 2,679,369 | 477,775 ± 147,251 | 0.97 | 0.75 | 0.64 |
| CCL17 | 200 ± 457 | 421 ± 867 | 42 ± 44 | 0.76 | 0.82 | 0.42 |
| CCL22 | 61,812 ± 163,171 | 53,105 ± 113,545 | 4,306 ± 5,750 | 0.99 | 0.65 | 0.88 |
| TNFα | 75,044 ± 41,433 | 75,941 ± 43,938 | 130,385 ± 47,351 | 1.00 | 0.017 * | 0.10 |
| IL1β | 102,121 ± 37,416 | 107,456 ± 20,675 | 111,181 ± 25,317 | 0.98 | 0.88 | 0.99 |
| CCL20 | 16,033 ± 9,224 | 16,826 ± 7,375 | 16,231 ± 5,003 | 0.99 | 1.00 | 0.99 |
| ALOX15 | 18,741 ± 19,420 | 24,167 ± 19,036 | 142,494 ± 188,198 | 1.00 | 0.03 * | 0.16 |
| ALOX5 | 10,655,887 ± 2,754,206 | 1,1308,968 ± 2,851,849 | 11,033,153 ± 1,397,415 | 0.94 | 0.98 | 0.99 |
| ALOX5AP | 13,940,937 ± 3,209,466 | 12,710,464 ± 2,864,216 | 12,877,643 ± 2,812,301 | 0.83 | 0.80 | 1.00 |
| LTA4H | 8,532,533 ± 1,944,551 | 8,455,408 ± 1,191,877 | 7,859,076 ± 1,647,800 | 1.00 | 0.75 | 0.91 |
| LTC4S | 4,959 ± 3,748 | 5,445 ± 3,189 | 9,086 ± 4,988 | 0.99 | 0.07 | 0.33 |

Levels of expression of IL-13 were below the limit of detection (cycle threshold >40) in 26 of the 29 subjects, and IL-4 was below the limit of detection in 20 of the 29 subjects (no differences between the three groups for either cytokine, all p > 0.35). All other genes were within the limit of detection across samples.

### Example 9

### Relationship of “IL-13 High” and “IL-13 Low” Subphenotypes of Asthma to Serum Protein Biomarkers

Further microarray analysis led us to identify from the set of genes and probes listed in Table 4, a set of 35 probes representing 28 genes whose expression was co-regulated with periostin in individual subjects below a threshold false discovery rate (FDR) q-value of 0.05. These genes and probes and associated data are presented in Table 9. Hierarchical cluster analysis of all subjects, including healthy controls and asthmatics, based on expression levels of those probes confirmed and further defined the two major clusters described above of (1) a cluster with high expression levels of periostin and co-regulated genes and (2) a cluster with low expression levels of periostin and co-regulated genes (FIG. 11). Mast cell genes include RGS13, TPSG1, TPSAB1, FCER1B, CPA3 and SLC18A2. Eosinophil genes include include P2RY14 and ALOX15.

The cluster with high expression of periostin and co-regulated genes comprised 23 asthmatic subjects and 1 healthy control (FIG. 11, cluster 1, indicated in red) whereas the cluster with low expression of periostin and co-regulated genes comprised the remaining 19 asthmatics interspersed with 26 of the healthy controls (FIG. 11, cluster 2, indicated in green). In Example 8, we described clustering of subjects in this dataset based on the microarray-determined expression levels of three of these probes: 210809_s_at (periostin), 210107_at (CLCA1), and 204614_at (serpinB2). The three-probe signature described in Example 8 correlates well with this full 35-probe signature, differing for seven asthmatics and one healthy control (discrepant calls indicated in FIG. 11 with *).

TABLE 9
IL-13 gene signature genes and exemplary probes.
Microarray signal intensity

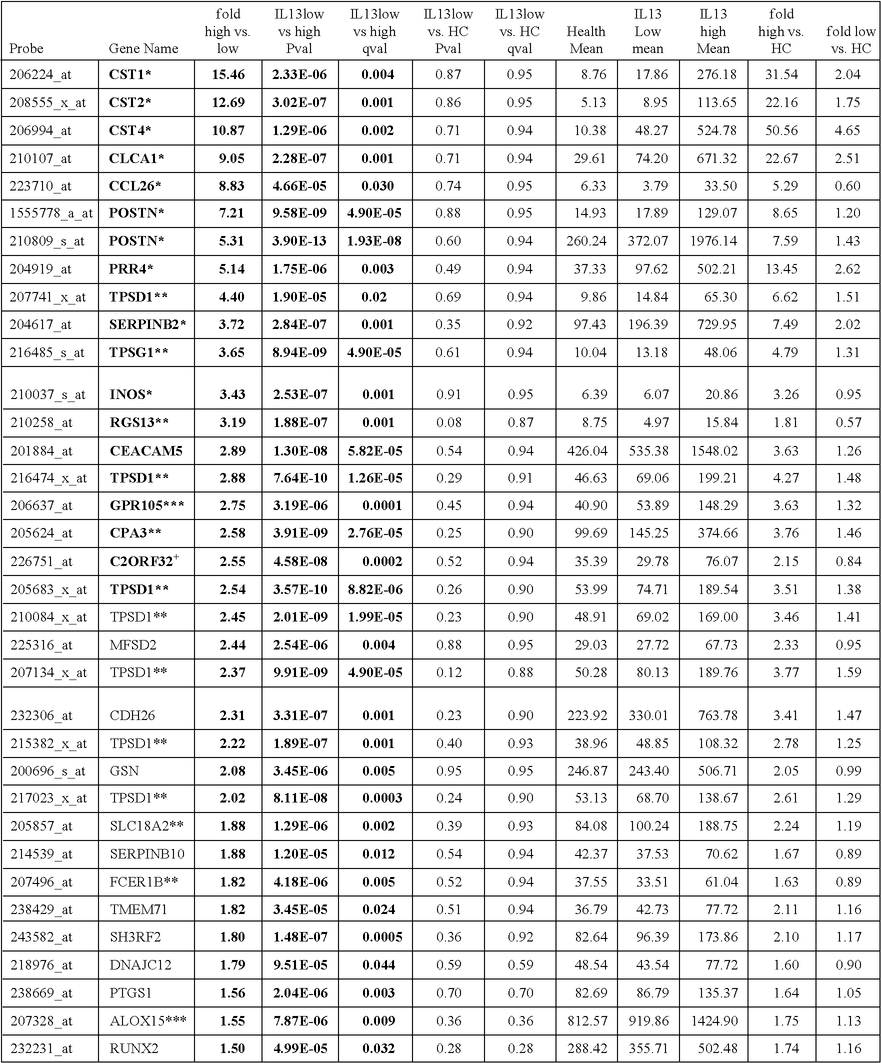

Probes are ranked in order of fold change in “IL-13 high” vs. “IL-13 low” asthmatics (third column from left); probes with a 2.5 fold or greater enrichment in “IL-13 high” asthma are shown with bolded gene names.
Probes corresponding to periostin (POSTN) and CEACAM5 are shaded.
Non mast cell genes > 3-fold upregulated in “IL-13 high” vs. “IL-13 low” asthma are indicated with a single asterisk (*).
Mast cell-specific genes are indicated with a double asterisk (**)
Eosinophil-specific genes are indicated with a triple asterisk (***).
(⁺Note
that based on clustering pattern, C2ORF32 signal is likely mast cell-derived).

Using the three-gene (periostin, CLCA1, and serpinB2) IL-13 signature, we showed in Example 8 that systemic markers of allergic inflammation including serum IgE and peripheral blood eosinophil levels were significantly elevated in “IL-13 high” subphenotype asthmatics relative to “IL-13 low” subphenotype asthmatics. However, there was significant overlap between the asthmatic groups for each of these metrics taken individually. In addition, neither serum IgE or peripheral blood eosinophil levels alone constitutes a non-invasive metric for predicting the airway IL-13 signature and associated “IL-13 high” or “IL-13 low” asthma subphenotype with simultaneous high sensitivity and specificity.

To determine whether the intersection of IgE and peripheral blood eosinophil levels could predict patterns of airway inflammation with greater accuracy than either metric alone, we evaluated serum IgE and peripheral blood eosinophil counts together versus airway IL-13 signature status. We found that, across the 42 asthmatics, serum IgE and peripheral blood eosinophil counts were correlated, albeit weakly (FIG. 4; data shown for the IL-4/13 signature; similar results were obtained for the IL-13 signature [see Table 10]). For the IL-13 signature, all of the “IL-13 high” asthmatics had eosinophil counts greater than 0.14×10⁹/L, but many of the “IL-13 low” asthmatics had lower eosinophil counts. All but two of the “IL-13 high” asthmatics had serum IgE levels greater than 100 IU/ml, but many “IL-13 low” asthmatics did not. The two metrics of (1) serum IgE ≧100 IU/ml and (2) eosinophil counts ≧0.14×10⁹/L combined yielded improved sensitivity and specificity for the IL-13 signature in the airway (Table 10). Thus, a composite of two commonly used peripheral blood metrics of allergic inflammation may be an effective noninvasive biomarker for airway IL-13 driven inflammation.

TABLE 10
Sensitivity, specificity, positive and negative predictive values of
IgE and peripheral blood eosinophil metrics for the IL-13 signature.

| IL-13 signature status | |
|---|---|
| High | Low |

Positive criteria:
serum IgE >100 IU/ml

| | | | | | |
|---|---|---|---|---|---|
| Test Result | + | 21 | 10 | Sensitivity: | 21/23 = 0.91 |
| | − | 2 | 9 | Specificity: | 9/19 = 0.47 |
| | | | | PPV: | 21/31 = 0.68 |
| | | | | NPV: | 9/11 = 0.82 |

Positive criteria:
eosinophils ≧0.14 ×10⁹/L

| | | | | | |
|---|---|---|---|---|---|
| Test Result | + | 23 | 11 | Sensitivity: | 23/23 = 1 |
| | − | 0 | 8 | Specificity: | 8/19 = 0.42 |
| | | | | PPV: | 23/34 = 0.68 |
| | | | | NPV: | 8/8 = 1 |

Positive criteria:
IgE >100 IU/ml AND eosinophils ≧0.14 × 10⁹/L

| | | | | | |
|---|---|---|---|---|---|
| Test Result | + | 21 | 5 | Sensitivity: | 21/23 = 0.91 |
| | − | 2 | 14 | Specificity: | 14/19 = 0.74 |
| | | | | PPV: | 21/26 = 0.81 |
| | | | | NPV: | 14/16 = 0.88 |

To identify additional systemic (noninvasive) candidate biomarkers of the bronchial epithelial IL-13 signature, we examined the signature for genes encoding extracellular or secreted proteins that might be detectable in peripheral blood. Three candidates of particular interest were CCL26, periostin, and CEACAM5. As CCL26 has been previously described as a Th2 cytokine-induced chemokine in bronchial epithelium [71], we focused on the characterization of periostin and CEACAM5, which have not previously been described as serum biomarkers of Th2 inflammation. CEACAM5 encodes carcinoembryonic antigen (CEA), which is a frequently used prognostic serum biomarker in epithelial-derived cancers. Periostin has also been described in a limited number of studies as a serum biomarker for certain cancers and, intriguingly, was detectable at a level in the range of 10s-100 s of ng/ml serum in most subjects, attractive characteristics for a serum marker to be readily detected by immunoassays.

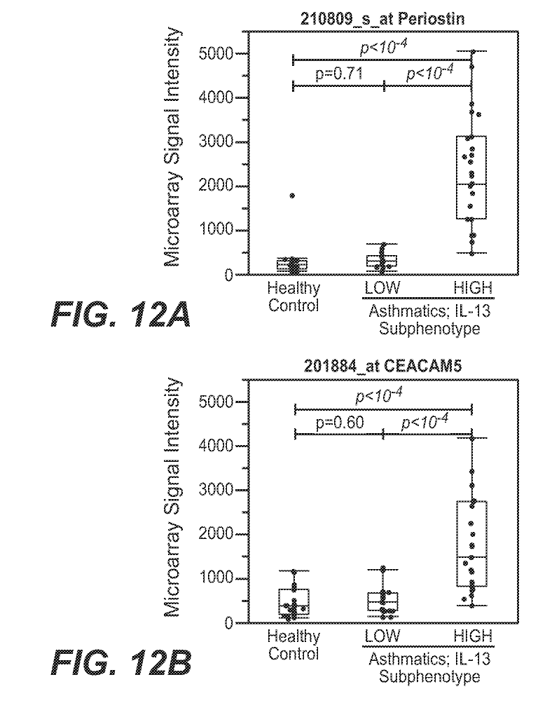

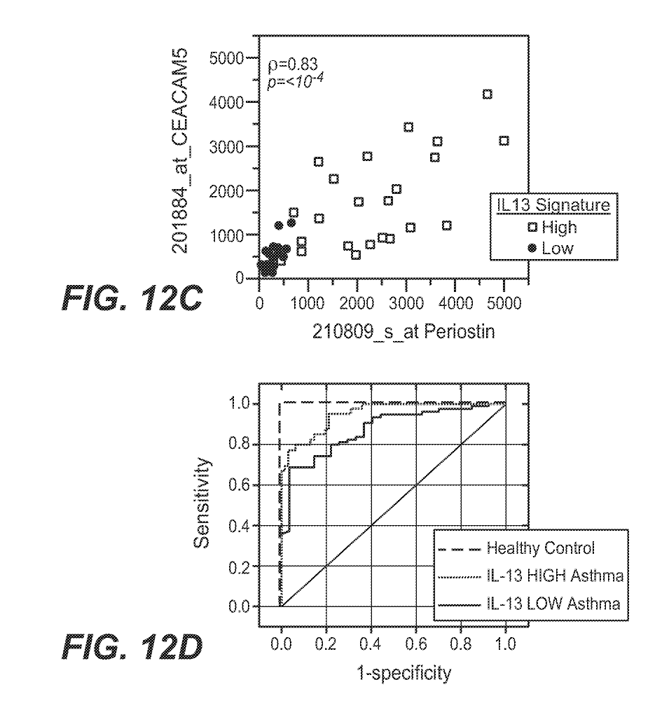

As shown in FIG. 12A-B, Periostin and CEACAM5 are each good individual representatives of the IL-13 signature, exhibiting significantly higher expression in “IL-13 high” asthmatics than in “IL-13 low” asthmatics or healthy controls. There was a strong correlation between microarray expression levels of periostin and CEACAM5 in individual asthmatics (FIG. 12C). To confirm these gene expression patterns and determine whether periostin and CEACAM5 expression could be used in an algorithm to distinguish “IL-13 high” asthmatics from “IL-13 low” asthmatics and healthy controls, we analyzed expression levels of the two genes by qPCR in the same bronchial epithelial brushing samples used for microarray analysis. There was a high degree of concordance between microarray and qPCR values in individual subjects (not shown). We used ordinal logistic regression analysis to generate a predictive model for the microarray-derived 35-probe IL-13 status using qPCR values for periostin and CEACAM5. The model's predictive value was highly significant (p<0.0001) and periostin and CEACAM5 parameter estimates each had a significant effect in the model (p<0.02 for CEACAM5; p<0.0001 for periostin). Receiver operating characteristic (ROC) curve analysis demonstrated perfect productivity for healthy control and very high sensitivity and specificity for “IL-13 high” and “IL-13 low” asthma (FIG. 12D). Taken together, these data show that bronchial epithelial expression levels of periostin and CEACAM5 are good surrogates for the overall IL-13 signature.

To determine whether elevated levels of soluble periostin and CEA proteins were detectable in peripheral blood, we examined periostin and CEA in sera from 100 asthmatics and 48 healthy controls using immunoassays. In addition, we measured IgE and YKL-40, a serum marker previously described to be elevated in some asthmatics [72], in these same sera. We observed significantly elevated levels of IgE, periostin, CEA, and YKL-40 in asthmatics relative to healthy controls (FIG. 13A-D). However, in all cases, there was substantial overlap in serum levels of each biomarker between groups. As shown in Example 8, inhaled corticosteroid (ICS) treatment reduces the bronchial epithelial expression of periostin in asthmatics that have elevated periostin at baseline (see also [8]). Of the 100 asthmatics whose serum we examined, 51 were taking inhaled corticosteroids (ICS) and 49 were not. When comparing asthmatics not on ICS and asthmatics on ICS, ICS-treated subjects had significantly lower median serum levels of IgE and CEA, and showed a trend for lower periostin levels, while YKL-40 levels were unchanged (FIG. 13E-H). Nevertheless, asthmatics on ICS had higher median serum levels of IgE, periostin, and CEA than healthy controls (Table 13). As shown in FIG. 4 and Table 10, 21/23 asthmatics positive for the bronchial epithelial IL-13 signature (“IL-13 high”) had serum IgE levels greater than 100 IU/ml, although a proportion of “IL-13 low” asthmatics also had elevated IgE. We found that serum periostin levels trended higher and CEA levels were significantly higher in asthmatics with IgE ≧100 IU/ml (N=68) than in asthmatics with IgE <100 IU/ml (N=32; FIG. 13I-J). However, serum YKL-40 levels were significantly lower in the high IgE group (FIG. 13K). As airway expression levels of periostin and CEACAM5 were highly correlated in “IL-13 high” asthmatics, we examined the correlation between serum periostin and CEA across all asthmatics (FIG. 13L). We found that serum periostin and CEA levels were significantly correlated with each other across the asthmatic population, and within asthmatics not on ICS or asthmatics with IgE ≧100 IU/ml but not in healthy controls, asthmatics on ICS, or asthmatics with IgE <100 IU/ml (Table 11). Taken together, these data suggest that periostin and CEA may be serum biomarkers of a bronchial epithelial IL-13 induced gene signature in asthmatics.

TABLE 11
Correlations between serum biomarkers.

| | | | |
|---|---|---|---|
| Variable | by Variable | Spearman ρ | P-value |

All subjects (Controls, N = 48; Asthmatics, N = 100)

| | | | |
|---|---|---|---|
| YKL40 (ng/ml) | IgE (IU/ml) | 0.0140 | 0.8661 |
| CEA (ng/ml) | IgE (IU/ml) | 0.4040 | [symbol] |
| CEA (ng/ml) | YKL40 (ng/ml) | 0.2935 | [symbol] |
| Periostin (ng/ml) | IgE (IU/ml) | 0.2259 | [symbol] |
| Periostin (ng/ml) | YKL40 (ng/ml) | 0.1253 | 0.1291 |
| Periostin (ng/ml) | CEA (ng/ml) | 0.3556 | [symbol] |

Healthy Controls (N = 48)

| | | | |
|---|---|---|---|
| YKL40 (ng/ml) | IgE (IU/ml) | 0.0420 | 0.7768 |
| CEA (ng/ml) | IgE (IU/ml) | −0.0996 | 0.5007 |
| CEA (ng/ml) | YKL40 (ng/ml) | 0.1914 | 0.1926 |
| Periostin (ng/ml) | IgE (IU/ml) | −0.2451 | 0.0931 |
| Periostin (ng/ml) | YKL40 (ng/ml) | 0.2246 | 0.1249 |
| Periostin (ng/ml) | CEA (ng/ml) | 0.4495 | [symbol] |

All Asthmatics (N = 100)

| | | | |
|---|---|---|---|
| YKL40 (ng/ml) | IgE (IU/ml) | −0.2144 | [symbol] |
| CEA (ng/ml) | IgE (IU/ml) | 0.3579 | [symbol] |
| CEA (ng/ml) | YKL40 (ng/ml) | 0.0890 | 0.3787 |
| Periostin (ng/ml) | IgE (IU/ml) | 0.3262 | [symbol] |
| Periostin (ng/ml) | YKL40 (ng/ml) | 0.0108 | 0.9152 |
| Periostin (ng/ml) | CEA (ng/ml) | 0.3530 | [symbol] |

Asthmatics; not on ICS (N = 49)

| | | | |
|---|---|---|---|
| YKL40 (ng/ml) | IgE (IU/ml) | −0.1198 | 0.4123 |
| CEA (ng/ml) | IgE (IU/ml) | 0.3727 | [symbol] |
| CEA (ng/ml) | YKL40 (ng/ml) | 0.1111 | 0.4471 |
| Periostin (ng/ml) | IgE (IU/ml) | 0.4236 | [symbol] |
| Periostin (ng/ml) | YKL40 (ng/ml) | 0.0186 | 0.8989 |
| Periostin (ng/ml) | CEA (ng/ml) | 0.4033 | [symbol] |

Asthmatics; on ICS (N = 51)

| | | | |
|---|---|---|---|
| YKL40 (ng/ml) | IgE (IU/ml) | −0.2553 | 0.0706 |
| CEA (ng/ml) | IgE (IU/ml) | 0.2251 | 0.1123 |
| CEA (ng/ml) | YKL40 (ng/ml) | 0.1035 | 0.4699 |
| Periostin (ng/ml) | IgE (IU/ml) | 0.1974 | 0.1650 |
| Periostin (ng/ml) | YKL40 (ng/ml) | 0.0783 | 0.5849 |
| Periostin (ng/ml) | CEA (ng/ml) | 0.2197 | 0.1213 |

Asthmatics; IgE <100 IU/ml (N = 32)

| | | | |
|---|---|---|---|
| CEA (ng/ml) | YKL40 (ng/ml) | 0.4003 | [symbol] |
| Periostin (ng/ml) | YKL40 (ng/ml) | 0.3513 | [symbol] |

All subjects (Controls, N = 48; Asthmatics, N = 100)

| | | | |
|---|---|---|---|
| Periostin (ng/ml) | CEA (ng/ml) | 0.1968 | 0.2802 |

Asthmatics; IgE ≧100 IU/ml (N = 68)

| | | | |
|---|---|---|---|
| CEA (ng/ml) | YKL40 (ng/ml) | 0.0370 | 0.7647 |
| Periostin (ng/ml) | YKL40 (ng/ml) | −0.1264 | 0.3043 |
| Periostin (ng/ml) | CEA (ng/ml) | 0.4145 | [symbol] |

Spearman's rank order correlation, ρ, is indicated with associated p-values for the correlations. Highly significant p-values (<0.05) are indicated in bold italics.

Within the IL-13 signature, we observed several functional groups of multiple genes, including genes encoding protease inhibitors and genes expressed in mast cells and eosinophils, which may represent infiltration into and/or anatomic localization of those cells to bronchial epithelium. Greater than 90% of cells in each bronchial brushing sample were bronchial epithelial cells or goblet cells (mean 97%, median 98%, minimum 91%), but very small numbers of infiltrating “contaminant” cells with cell-specific gene expression patterns were detectable in the microarrays. Mast cell specific genes included tryptases (TPSAB1 [TPSD1] and TPSG1), CPA3, FCER1B, RGS13, and SLC18A2 [73, 74]. Also clustering tightly with mast cell genes was CNRIP1 (C2ORF32), a cannabinoid receptor-interacting GTPase. Given the well-established role of cannabinomimetics in the regulation of mast cell function [75], it is likely that CNRIP1 represents a mast cell-specific gene as well. Given the significant role of tissue-resident mast cells in allergic disease and the recent observation that the presence of IL-13 expressing mast cells in asthmatic endobronchial biopsy specimens is positively correlated with detectable levels of IL-13 in sputum [6], the high correlation between mast cell-specific genes and the IL-13 signature suggests that: 1) mast cells may be a significant source of IL-13 in the airway epithelium and 2) mast cell infiltration into airway epithelium may be a unique feature of the “IL-13 high” subset of asthmatics. Eosinophil specific genes include P2RY14 (GPR105) and ALOX15, although in Example 8 we described ALOX15 expression in alveolar macrophages from asthmatics.

Multiple probes corresponding to serine and cysteine protease inhibitors were present in the IL-13 signature, including Serpins B2 and B10, and cystatins (CST) 1, 2, and 4. SerpinB2 is a member of a large family of serine protease inhibitors encoded in a gene cluster on chromosome 18q21. Expression levels of Serpins B2 [8], B3, and B4 are induced in airway epithelial cells upon stimulation by recombinant IL-4 and IL-13 [7, 15]. Cystatins (CST) 1, 2, and 4 are members of a large family of cysteine protease inhibitors encoded in a gene cluster on chromosome 20p11. Several cystatins are expressed in bronchial epithelium [16]; CST4 has been identified at elevated levels in bronchoalveolar lavage fluid (BAL) of asthmatics [17]; serum CST3 is elevated in asthmatics relative to healthy controls and its levels are decreased by ICS treatment [18]. As serpin and CST gene families are each colocalized on the chromosome, we explored whether any additional members of the serpin and cystatin gene families are co-regulated with those already identified. We performed hierarchical clustering of the microarray data across all subjects, restricted to serpin and cystatin gene families. We found that, out of over 40 protease inhibitor genes represented on the array, only serpins B2, B4, and B10; and cystatins 1, 2, and 4 were significantly co-regulated, with the highest expression levels occurring in asthmatics having the “IL-13 high” signature (FIG. 2B and Table 12). As many aeroallergens possess protease activity and protease-activated receptors (PARs) are associated with the activation of allergic inflammatory cascades [76], the upregulation of protease inhibitors by Th2 cytokines may represent a compensatory response to protease-containing aeroallergens.

TABLE 12
Probe IDs of Serpin and CST genes used for clustering in FIG. 2B.

| Probe ID | Gene Name | Probe ID | Gene Name | Probe ID | Gene Name |
|---|---|---|---|---|---|
| 205075_at | SERPINF2 | 236599_at | SERPINE2 | 200986_at | SERPING1 |
| 206595_at | CST6 | 233968_at | CST11 | 1555551_at | SERPINB5 |
| 206325_at | SERPINA6 | 1554616_at | SERPINB8 | 233797_s_at | CST11 |
| 206421_s_at | SERPINB7 | 213874_at | SERPINA4 | 210140_at | CST7 |
| 227369_at | SERBP1 | 220627_at | CST8 | 209720_s_at | SERPINB3 |
| 206034_at | SERPINB8 | 1568765_at | SERPINE1 | 209719_x_at | SERPINB3 |
| 202376_at | SERPINA3 | 206386_at | SERPINA7 | 210413_x_at | SERPINB4 |
| 207636_at | SERPINI2 | 202627_s_at | SERPINE1 | 208531_at | SERPINA2 |
| 1552544_at | SERPINA12 | 1554491_a_at | SERPINC1 | 209723_at | SERPINB9 |
| 231248_at | CST6 | 210076_x_at | SERBP1 | 212190_at | SERPINE2 |
| 1553057_at | SERPINB12 | 217725_x_at | SERBP1 | 211361_s_at | SERPINB13 |
| 240177_at | CST3 | 217724_at | SERBP1 | 217272_s_at | SERPINB13 |
| 202628_s_at | SERPINE1 | 236449_at | CSTB | 204855_at | SERPINB5 |
| 216258_s_at | SERPINB13 | 207714_s_at | SERPINH1 | 209725_at | UTP20 |
| 210049_at | SERPINC1 | 202283_at | SERPINF1 | 214539_{—}at | SERPINB10 |
| 220626_at | SERPINA10 | 211474_s_at | SERPINB6 | 204614_{—}at | SERPINB2 |
| 209443_at | SERPINA5 | 209669_s_at | SERBP1 | 208555_{—}x_{—}at | CST2 |
| 209722_s_at | SERPINB9 | 1556950_s_at | SERPINB6 | 206224_{—}at | CST1 |
| 202834_at | SERPINA8 | 228129_at | SERBP1 | 206994_{—}at | CST4 |
| 205352_at | SERPINI1 | 201201_at | CSTB | 211906_{—}s_{—}at | SERPINB4 |
| 211362_s_at | SERPINB13 | 213572_s_at | SERPINB1 | 230318_at | SERPINA1 |
| 205576_at | SERPIND1 | 212268_at | SERPINB1 | 201360_at | CST3 |
| 1554386_at | CST9 | 1552463_at | SERPINB11 | 210466_s_at | SERBP1 |
| 242814_at | SERPINB9 | 202833_s_at | SERPINA1 | 204971_at | CSTA |
| 239213_at | SERPINB1 | 211429_s_at | SERPINA1 | | |
| 230829_at | CST9L | | | | |

Probes are listed in order (top to bottom, left to right) found on heatmap at left of FIG. 2B. Probes clustering with IL-13 signature genes are indicated in bold.

The mouse orthologue of CLCA1, mCLCA3 (also known as gob-5) has been previously identified as a gene associated with goblet cell metaplasia of airway epithelium and mucus production; both are induced by Th2 cytokines including IL-9 and IL-13 [12-14]. PRR4 is a member of a large gene family encoded in a cluster on chromosome 12p13. These genes encode proline-rich proteins, which are found in mucosal secretions including saliva and tears. Related, but non-orthologous proteins SPRR1a, 2a, and 2b have been identified in bronchial epithelium in a mouse model of asthma and are induced by IL-13 [19, 20]. Proline-rich proteins from the PRR/PRB family have been identified in bronchial secretions [21] and their expression has been documented in bronchial epithelium [16]. CCL26 (Eotaxin-3) is a well-documented IL-4 and IL-13 inducible eosinophil attracting chemokine in asthmatic airway epithelium [71]. CDH26 is a cadherin-like molecule of unknown function that has recently been identified in a microarray analysis of eosinophilic esophagitis [11]. That study identified several additional genes overlapping with our bronchial epithelial IL-13 signature including periostin, SerpinB4, and CCL26 [11]. As CDH26 is corrugated with eotaxins and overexpressed in diseases characterized by eosinophilic inflammation, it is tempting to speculate that CDH26 plays a role in eosinophil infiltration into mucosal tissues. Inducible nitric oxide synthase (iNOS) is associated with airway inflammation and is induced by IL-13 in human primary bronchial epithelial cell cultures [23]. The measurement of exhaled nitric oxide (eNO) is commonly used in the diagnosis and monitoring of asthma. Considered together, many of the genes described here as components of the IL-13 signature are highly consistent with in vitro and animal models of Th2 inflammation and play plausible roles in Th2-driven pathology in human asthma.

TABLE 13
Levels of serum biomarkers.

| | Healthy Control | Asthma | |
|---|---|---|---|
| | (N = 48) | (N = 100) | P-value |
| IgE (IU/ml) | 63 (0-590) | 234 (1-2098) | <0.0001 |
| Periostin (ng/ml) | 38 (0-139) | 52 (0-117) | 0.03 |
| CEA (ng/ml) | <0.2 (<0.2-5.5) | 2 (<0.2-21*) | <0.0001 |
| YKL-40 (ng/ml) | 48 (18-265) | 64 (19-494) | 0.0004 |

Effect of inhaled corticosteroid treatment
on serum biomarkers in asthmatics

| | No ICS | ICS | |
|---|---|---|---|
| | (N = 49) | (N = 51) | P-value |
| IgE (IU/ml) | 322 (8-1395) | 132 (1-2098) | 0.011 |
| Periostin (ng/ml) | 54 (0-110) | 48 (0-117) | 0.07 |
| CEA (ng/ml) | 2.5 (<0.2-7.5) | 1.9 (<0.2-21*) | 0.041 |
| YKL-49 (ng/ml) | 62 (19-353) | 72 (24-494) | 0.30 |

Levels of serum biomarkers in asthmatics by IgE level category

| | IgE <100 IU/ml | IgE ≧100 IU/ml | |
|---|---|---|---|
| | (N = 32) | (N = 68) | P-value |
| CEA (ng/ml) | 1.6 (<0.2-7.5) | 2.5 (<0.2-21*) | 0.031 |
| Periostin (ng/ml) | 49 (0-117) | 57 (0-112) | 0.20 |
| YKL-40 (ng/ml) | 83 (19-494) | 61 (23-290) | 0.01 |

Values shown as median (range)
p-values are Wilcoxon rank rank sum
*99/100 asthmatics had CEA values ≦7.5 ng/ml

CEACAM5 encodes a cell-surface glycoprotein found in many epithelial tissues and elevated serum CEACAM5 (carcinoembryonic antigen; CEA) is a well-documented systemic biomarker of epithelial malignancies and metastatic disease. Elevated CEA levels have been reported in a subset of asthmatics, with particularly high serum levels observed in asthmatics with mucoid impaction [75]. Intriguingly, while the upper limit of normal for serum CEA is in the 2.5-3 ng/ml range, the lower limit for suspicion of malignancy is 10 ng/ml. In our analyses, we find that over 95% of healthy controls had CEA levels below 3 ng/ml while ⅓ of asthmatics had CEA levels between 3 and 7.5 ng/ml, and of these, the vast majority had serum IgE levels above 100 IU/ml. This suggests that a robust window of detection for CEA may be present in asthmatics with Th2-driven airway inflammation. Periostin has been described as an IL-4 and IL-13 inducible gene in asthmatic airways [7-9, 77] as a gene upregulated in epithelial-derived cancers that may be associated with invasiveness and extracellular matrix change [64-67], and whose serum protein levels are detectable and elevated in some cancers [68-70]. As it may play a role in eosinophilic tissue infiltration in eosinophilic esophagitis [11, 77], periostin could be an important factor in, and biomarker of, eosinophilic diseases such as Th2-driven asthma.

The standard of care for bronchial asthma that is not well-controlled on symptomatic therapy (i.e. β-agonists) is inhaled corticosteroids (ICS). In mild-to-moderate asthmatics with elevated levels of IL-13 in the airway [6] and eosinophilic esophagitis patients with elevated expression levels of IL-13 in esophageal tissue [11], ICS treatment substantially reduces the level of IL-13 and IL-13-induced genes in the affected tissues. In airway epithelium of asthmatics after one week of ICS treatment and in cultured bronchial epithelial cells, we have shown that corticosteroid treatment substantially reduces IL-13-induced expression levels of periostin, serpinB2, and CLCA1 [8]. Further examination of the genes listed in Table 9 revealed that, in the 19 subjects in our study who received one week of ICS treatment prior to a second bronchoscopy, the vast majority of IL-13 signature genes was significantly downregulated by ICS treatment in asthmatic bronchial airway epithelium. This downregulation could be the result of ICS-mediated reduction of IL-13 levels, ICS-mediated reduction of target gene expression, or a combination of the two. In severe asthmatics who are refractory to ICS treatment, a similar fraction of subjects (approximately 40%) was found to have detectable sputum IL-13 levels to that seen in mild, ICS-naïve asthmatics [6], which is comparable to the fraction of subjects with the IL-13 signature observed in this study. This observation suggests that, although the IL-13 signature is significantly downregulated by ICS treatment in the mild-moderate, ICS-responsive asthmatics examined in the present study, it may still be present in severe steroid-resistant asthmatics. Similar observations have been reported for eosinophilic inflammation in bronchial biopsies [78] and persistence of IL-4 and IL-5 expressing cells in BAL [79] of steroid-refractory asthmatics. There is currently a large number of biological therapeutics in clinical development directed against IL-13 or related factors in Th2 inflammation [50, 80], including, without limitation, those described herein. Our findings suggest that only a fraction of steroid-naëve mild-to-moderate asthmatics may have activity of this pathway, and given its susceptibility to ICS treatment, it is likely that a smaller fraction of moderate-to-severe, steroid-refractory asthmatics has activity of this pathway. Therefore, biomarkers that identify asthmatics likely to have IL-13 driven inflammation in their airways may aid in the identification and selection of subjects most likely to respond to these experimental targeted therapies.

We claim:

1. A method of diagnosing an asthma subtype in a patient comprising measuring the gene expression of any one or combination of genes selected from the group of consisting of POSTN, CST1, CST2, CCL26, CLCA1, PRB4, PRB4, SERPINB2, CEACAM5, iNOS, SERPINB4, CST4, and SERPINB10, wherein elevated expression levels of any one, combination or all of said genes is indicative of the asthma subtype.

2. The method according to claim 1, further comprising the genes PRB4, TPSD1, TPSG1, MFSD2, CPA3, GPR105, CDH26, GSN, C2ORF32, TRACH2000196 (TMEM71), DNAJC12, RGS13, SLC18A2, SH3RF2, FCER1B, RUNX2, PTGS1, and ALOX15.

3. The method according to claim 1, wherein gene expression is measured by assaying for protein or mRNA levels.

4. The method according to claim 3, wherein the mRNA levels are measured by using a PCR method or a microarray chip.

5. The method according to claim 4, wherein the PCR method is qPCR.

6. The method according to claim 3, wherein the mRNA levels of the gene of interest relative to a control gene mRNA levels greater than 2.5 fold is indicative of the asthma subtype.

7. A method of diagnosing an asthma subtype in a patient comprising measuring any one of the biomarkers from a patient sample selected from the group consisting of: serum total IgE levels, serum CEA levels, serum periostin levels, peripheral blood eosinophils and bronchoalveolar lavage (BAL) eosinophils, wherein elevated levels of CEA, serum periostin, peripheral blood eosinophils and bronchoalveolar lavage (BAL) eosinophils is indicative of the asthma subtype.

8. The method according to claim 7, wherein an IgE level greater than 100 IU/ml is indicative of the asthma subtype.

9. The method according to claim 7, wherein a peripheral blood eosinophil level greater than 0.14×10e9/L is indicative of the asthma subtype.

10. A method of diagnosing an asthma subtype in a patient comprising measuring the ratio of Muc5AC:MUC5B mRNA or the ratio of Muc5AC:MUC5B protein from a sample of an asthma patient, wherein a ratio greater than 25 is indicative of the asthma subtype.

11. The method according to claim 10, wherein the sample is obtained from an epithelial brushing.

12. The method according to claim 10, wherein the sample comprises airway epithelial cells.

13. A method of treating asthma comprising administering a therapeutic agent to a patient expressing elevated levels of any one or combination of the genes selected from the group consisting of POSTN, CST1, CST2, CCL26, CLCA1, PRR4, PRB4, SERPINB2, CEACAM5, iNOS, SERPINB4, CST4, and SERPINB10.

14. The method according to claim 13, further comprising the genes PRB4, TPSD1, TPSG1, MFSD2, CPA3, GPR105, CDH26, GSN, C2ORF32, TRACH2000196 (TMEM71), DNAJC12, RGS13, SLC18A2, SH3RF2, FCER1B, RUNX2, PTGS1, and ALOX15.

15. A method of treating asthma comprising administering a therapeutic agent to a patient expressing elevated levels of serum total IgE, serum CEA, serum periostin, peripheral blood eosinophils and/or bronchoalveolar lavage (BAL) eosinophils.

16. A method of treating asthma comprising administering a therapeutic agent to a patient having a ratio of Muc5AC:MUC5B mRNA or ratio of Muc5AC:MUC5B protein greater than 25 in a patient sample.

17. The method according to any one of claims 13-16, wherein the patient to be treated is a mild-to-moderate, steroid-naive asthma patient.

18. The method according to any one of claims 13-16, wherein the patient to be treated is a moderate-to-severe, steroid-resistant asthma patient.

19. The method according to any one of claims 13-16, wherein the patient has asthma induced by the TH2 pathway.

20. The method according to any one of claims 13-16, wherein the patient has been diagnosed according to the method of any one of the aforementioned claims.

21. The method according to any one of claims 13-16, wherein the therapeutic agent is selected from the group consisting of an agent that binds to a target selected from the group consisting of: IL-9, IL-5, IL-13, IL-4, OX40L, TSLP, IL-25, IL-33 and IgE; and receptors such as: IL-9 receptor, IL-5 receptor, IL-4receptor alpha, IL-13receptoralpha1 and IL-13receptoralpha2, OX40, TSLP-R, IL-7Ralpha, IL17RB, ST2, CCR3, CCR4, CRTH2, FcepsilonRI and FcepsilonRII/CD23.

22. The method according to any one of claims 13-16, wherein the therapeutic agent is an immunoadhesin, a peptibody or an antibody.

23. A method of treating asthma comprising administering a therapeutic agent to an asthma patient not expressing elevated levels of any one or combination of the genes selected from the group consisting of POSTN, CST1, CST2, CCL26, CLCA1, PRR4, PRB4, SERPINB2, CEACAM5, iNOS, SERPINB4, CST4, and SERPINB10.

24. The method according to claim 23, further comprising the genes PRB4, TPSD1, TPSG1, MFSD2, CPA3, GPR105, CDH26, GSN, C20RF32, TRACH2000196 (TMEM71), DNAJC12, RGS13, SLC18A2, SH3RF2, FCER1B, RUNX2, PTGS1, and ALOX15.

25. A method of treating asthma comprising administering a therapeutic agent to an asthma patient not expressing elevated levels of serum total IgE levels, serum CEA levels, serum periostin levels, peripheral blood eosinophils and/or bronchoalveolar lavage (BAL) eosinophils.

26. A method of treating asthma comprising administering a therapeutic agent to an asthma patient not having a Muc5AC:MUC5B mRNA or protein ratio greater than 25 in a patient sample.

27. The method according to claim 26, wherein the therapeutic agent is an IL-17 pathway inhibitor.

28. A kit for diagnosing an asthma subtype in a patient comprising (1) one or more nucleic acid molecules that hybridize with a gene, wherein the gene is selected from the group of consisting of POSTN, CST1, CST2, CCL26, CLCA1, PRR4, PRB4, SERPINB2, CEACAM5, iNOS, SERPINB4, CST4, and SERPINB10 and (2) instructions for measuring the expression levels of the gene from a patient sample, wherein the elevated expression levels of any one, combination or all of said genes is indicative of the asthma subtype.

29. The kit according to claim 28, further comprising a gene selected from the group consisting of: PRB4, TPSD1, TPSG1, MFSD2, CPA3, GPR105, CDH26, GSN, C20RF32, TRACH2000196 (TMEM71), DNAJC12, RGS13, SLC18A2, SH3RF2, FCER1B, RUNX2, PTGS1, and ALOX15.

30. The kit according to claim 28, wherein gene expression is measured by assaying for mRNA levels.

31. The kit according to claim 30, wherein the assay comprises a PCR method or the use of a microarray chip.

32. The kit according to claim 31, wherein the PCR method is qPCR.

33. The kit according to claim 30, wherein the mRNA levels of the gene of interest relative to a control gene mRNA level greater than 2.5 fold is indicative of the asthma subtype.

34. A kit for diagnosing an asthma subtype in a patient comprising (1) one or more protein molecules that bind to a protein selected from the group of consisting of POSTN, CST1, CST2, CCL26, CLCA1, PRR4, PRB4, SERPINB2, CEACAM5, iNOS, SERPINB4, CST4, and SERPINB10 and (2) instructions for measuring the expression levels of the protein from a patient sample, wherein the elevated expression levels of any one, combination or all of said proteins is indicative of the asthma subtype.

35. The kit according to claim 10, further comprising a protein is selected from the group consisting of: PRB4, TPSD1, TPSG1, MFSD2, CPA3, GPR105, CDH26, GSN, C20RF32, TRACH2000196 (TMEM71), DNAJC12, RGS13, SLC18A2, SH3RF2, FCER1B, RUNX2, PTGS1, and ALOX15.

36. The kit according to claim 34, wherein the assay comprises the use of a microarray chip comprising the protein molecules.

37. A kit for diagnosing an asthma subtype in a patient comprising instructions for measuring any one of the biomarkers from a patient sample selected from the group consisting of: serum total IgE levels, serum CEA levels, serum periostin levels, peripheral blood eosinophils and bronchoalveolar lavage (BAL) eosinophils, wherein elevated levels of CEA, serum periostin, peripheral blood eosinophils and bronchoalveolar lavage (BAL) eosinophils.

38. The kit according to claim 37, wherein an IgE level greater than 100 IU/ml is indicative of the asthma subtype.

39. The kit according to claim 37, wherein a peripheral blood eosinophil level greater than 0.14×10e9/L is indicative of the asthma subtype.

40. A kit for diagnosing an asthma subtype in a patient comprising instructions for measuring the ratio of Muc5AC:MUC5B mRNA or protein from a sample of an asthma patient, wherein a ratio greater than 25 is indicative of the asthma subtype.

41. The kit according to claim 40, wherein the sample is obtained from an epithelial brushing.

42. The kit according to claim 40, wherein the sample comprises airway epithelial cells.


SEQUENCE LISTING (261 sequences)

SEQ ID NO: 1 - protein, 836 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYTTKIITKVVEPKIKVIEGSLQPIIKTEGPTLTKVKIEGEPEFRLIKEGETITEVIHGEPIIKKYTKIIDGVPVEITEKETREERIITGPEIKYTRISTGGGETEETLKKLLQEEVTKVTKFIEGGDGHLFEDEEIKRLLQGDTPVRKLQANKKVQGSRRRLREGRSQ

SEQ ID NO: 2 - protein, 141 aa, Homo sapiens
MAQHLSTLLLLLATLAVALAWSPKEEDRIIPGGIYNADLNDEWVQRALHFAISEYNKATKDDYYRRPLRVLRARQQTVGGVNYFFDVEVGRTICTKSQPNLDTCAFHEQPELQKKQLCSFEIYEVPWENRRSLVKSRCQES

SEQ ID NO: 3 - protein, 94 aa, Homo sapiens
MMGLSLASAVLLASLLSLHLGTATRGSDISKTCCFQYSHKPLPWTWVRSYEFTSNSCSQRAVIFTTKRGKKVCTHPRKKWVQKYISLLKTPKQL

SEQ ID NO: 4 - protein, 914 aa, Homo sapiens
MGPFKSSVFILILHLLEGALSNSLIQLNNNGYEGIVVAIDPNVPEDETLIQQIKDMVTQASLYLFEATGKRFYFKNVAILIPETWKTKADYVRPKLETYKNADVLVAESTPPGNDEPYTEQMGNCGEKGERIHLTPDFIAGKKLAEYGPQGKAFVHEWAHLRWGVFDEYNNDEKFYLSNGRIQAVRCSAGITGTNVVKKCQGGSCYTKRCTFNKVTGLYEKGCEFVLQSRQTEKASIMFAQHVDSIVEFCTEQNHNKEAPNKQNQKCNLRSTWEVIRDSEDFKKTTPMTTQPPNPTFSLLQIGQRIVCLVLDKSGSMATGNRLNRLNQAGQLFLLQTVELGSWVGMVTFDSAAHVQSELIQINSGSDRDTLAKRLPAAASGGTSICSGLRSAFTVIRKKYPTDGSEIVLLTDGEDNTISGCFNEVKQSGAIIHTVALGPSAAQELEELSKMTGGLQTYASDQVQNNGLIDAFGALSSGNGAVSQRSIQLESKGLTLQNSQWMNGTVIVDSTVGKDTLFLITWTTQPPQILLWDPSGQKQGGFVVDKNTKMAYLQIPGIAKVGTWKYSLQASSQTLTLTVTSRASNATLPPITVTSKTNKDTSKFPSPLVVYANIRQGASPILRASVTALIESVNGKTVTLELLDNGAGADATKDDGVYSRYFTTYDTNGRYSVKVRALGGVNAARRRVIPQQSGALYIPGWIENDEIQWNPPRPEINKDDVQHKQVCFSRTSSGGSFVASDVPNAPIPDLFPPGQITDLKAEIHGGSLINLTWTAPGDDYDHGTAHKYIIRISTSILDLRDKFNESLQVNTTALIPKEANSEEVFLFKPENITFENGTDLFIAIQAVDKVDLKSEISNIARVSLFIPPQTPPETPSPDETSAPCPNIHINSTIPGIHILKIMWKWIGELQLSIA

SEQ ID NO: 5 - protein, 141 aa, Homo sapiens
MAWPLCTLLLLLATQAVALAWSPQEEDRIIEGGIYDADLNDERVQRALHFVISEYNKATEDEYYRRLLRVLRAREQIVGGVNYFFDIEVGRTICTKSQPNLDTCAFHEQPELQKKQLCSFQIYEVPWEDRMSLVNSRCQEA

SEQ ID NO: 6 - protein, 134 aa, Homo sapiens [sequence omitted]

SEQ ID NO: 7 - protein, 415 aa, Homo sapiens
MEDLCVANTLFALNLFKHLAKASPTQNLFLSPWSISSTMAMVYMGSRGSTEDQMAKVLQFNEVGANAVTPMTPENFTSCGFMQQIQKGSYPDAILQAQAADKIHSSFRSLSSAINASTGNYLLESVNKLFGEKSASFREEYIRLCQKYYSSEPQAVDFLECAEEARKKINSWVKTQTKGKIPNLLPEGSVDGDTRMVLVNAVYFKGKWKTPFEKKLNGLYPFRVNSAQRTPVQMMYLREKLNIGYIEDLKAQILELPYAGDVSMFLLLPDEIADVSTGLELLESEITYDKLNKWTSKDKMAEDEVEVYIPQFKLEEHYELRSILRSMGMEDAFNKGRANFSGMSERNDLFLSEVFHQAMVDVNEEGTEAAAGTGGVMTGRTGHGGPQFVADHPFLFLIMHKITNCILFFGRFSSP

SEQ ID NO: 8 - protein, 702 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 9 - protein, 1153 aa, Homo sapiens
MACPWKFLFKTKFHQYAMNGEKDINNNVEKAPCATSSPVTQDDLQYHNLSKQQNESPQPLVETGKKSPESLVKLDATPLSSPRHVRIKNWGSGMTFQDTLHHKAKGILTCRSKSCLGSIMTPKSLTRGPRDKPTPPDELLPQAIEFVNQYYGSFKEAKIEEHLARVEAVTKEIETTGTYQLTGDELIFATKQAWRNAPRCIGRIQWSNLQVFDARSCSTAREMFEHICRHVRYSTNNGNIRSAITVFPQRSDGKHDFRVWNAQLIRYAGYQMPDGSIRGDPANVEFTQLCIDLGWKPKYGRFDVVPLVLQANGRDPELFEIPPDLVLEVAMEHPKYEWFRELELKWYALPAVANMLLEVGGLEFPGCPFNGWYMGTEIGVRDFCDVQRYNILEEVGRRMGLETHKLASLWKDQAVVEINIAVLHSFQKQNVTIMDHHSAAESFMKYMQNEYRSRGGCPADWIWLVPPMSGSITPVFHQEMLNYVLSPFYYYQVEAWKTHVWQDEKRRPKRREIPLKVLVKAVLFACMLMRKTMASRVRVTILFATETGKSEALAWDLGALFSCAFNPKVVCMDKYRLSCLEEERLLLVVTSTFGNGDCPGNGEKLKKSLFMLKELNNKFRYAVFGLGSSMYPRFCAFAHDIDQKLSHLGASQLTPMGEGDELSGQEDAFRSWAVQTFKAACETFDVRGKQHIQIPKLYTSNVTWDPHHYRLVQDSQPLDLSKALSSMHAKNVFTMRLKSRQNLQSPTSSRATILVELSCEDGQGLNYLPGEHLGVCPGNQPALVQGILERVVDGPTPHQTVRLEALDESGSYWVSDKRLPPCSLSQALTYFLDITTPPTQLLLQKLAQVATEEPERQRLEALCQPSEYSKWKFTNSPTFLEVLEEFPSLRVSAGFLLSQLPILKPRFYSISSSRDHTPTEIHLTVAVVTYHTRDGQGPLHHGVCSTWLNSLKPQDPVPCFVRNASGFHLPEDPSHPCILIGPGTGIAPFRSFWQQRLHDSQHKGVRGGRMTLVFGCRRPDEDHIYQEEMLEMAQKGVLHAVHTAYSRLPGKPKVYVQDILRQQLASEVLRVLHKEPGHLYVCGDVRMARDVAHTLKQLVAAKLKLNEEQVEDYFFQLKSQKRYHEDIFGAVFPYEAKKDRVAVQPSSLEMSAL

SEQ ID NO: 10 - protein, 390 aa, Homo sapiens
MNSLSEANTKFMFDLFQQFRKSKENNIFYSPISITSALGMVLLGAKDNTAQQISKVLHFDQVTENTTEKAATYHVDRSGNVHHQFQKLLTEFNKSTDAYELKIANKLFGEKTYQFLQEYLDAIKKFYQTSVESTDFANAPEESRKKINSWVESQTNEKIKNLFPDGTIGNDTTLVLVNAIYFKGQWENKFKKENTKEEKFWPNKNTYKSVQMMRQYNSFNFALLEDVQAKVLEIPYKGKDLSMIVLLPNEIDGLQKLEEKLTAEKLMEWTSLQNMRETCVDLHLPRFKMEESYDLKDTLRTMGMVNIFNGDADLSGMTWSHGLSVSKVLHKAFVEVTEEGVEAAAATAVVVVELSSPSTNEEFCCNHPFLFFIRQNKTNSILFYGRFSSP

SEQ ID NO: 11 - protein, 141 aa, Homo sapiens
MARPLCTLLLLMATLAGALASSSKEENRIIPGGIYDADLNDEWVQRALHFAISEYNKATEDEYYRRPLQVLRAREQTFGGVNYFFDVEVGRTICTKSQPNLDTCAFHEQPELQKKQLCSFEIYEVPWEDRMSLVNSRCQEA

SEQ ID NO: 12 - protein, 393 aa, Homo sapiens
MLLILLSVALLALSSAQNLNEDVSQEESPSLIAGKPQGPPPQGGNQPQGPPPPPGKPQGPPPQGGNKPQGPPPPGKPQGPPPQGDKSRSPRSPPGKPQGPPPQGGNQPQGPPPPPGKPQGPPPQGGNKPQGPPPPGKPQGPPPQGDNKSRSSRSPPGKPQGPPPQGGNQPQGPPPPPGKPQGPPPQGGNKPQGPPPPGKPQGPPPQGDKSRSPRSPPGKPQGPPPQGGNQPQGPPPPPGKPQGPPPQGGNRPQGPPPPGKPQGPPPQGDKSRSSQSPPGKPQGPPPQGGNQPQGPPPPPGKPQGPPPQGGNKPQGPPPPGKPQGPPAQGGSKSQSARSPPGKPQGPPQQEGNNPQGPPPPAGGNPQQPQAPPAGQPQGPPRPPQGGRPSRPPQ

SEQ ID NO: 13 - protein, 235 aa, Homo sapiens
MLSLLLLALPVLASPAYVAPAPGQALQQTGIVGGQEAPRSKWPWQVSLRVRGPYWMHFCGGSLIHPQWVLTAAHCVEPDIKDLAALRVQLREQHLYYQDQLLPVSRIIVHPQFYIIQTGADIALLELEEPVNISSHIHTVTLPPASETFPPGMPCWVTGWGDVDNNVHLPPPYPLKEVEVPVVENHLCNAEYHTGLHTGHSFQIVRDDMLCAGSENHDSCQGDSGGPLVCKVNGT

SEQ ID NO: 14 - protein, 321 aa, Homo sapiens
MALGACGLLLLLAVPGVSLRTLQPGCGRPQVSDAGGRIVGGHAAPAGAWPWQASLRLRRMHVCGGSLLSPQWVLTAAHCFSGSLNSSDYQVHLGELEITLSPHFSTVRQIILHSSPSGQPGTSGDIALVELSVPVTLSSRILPVCLPEASDDFCPGIRCWVTGWGYTREGEPLPPPYSLREVKVSVVDTETCRRDYPGPGGSILQPDMLCARGPGDACQDDSGGPLVCQVNGAWVQAGIVSWGEGCGRPNRPGVYTRVPAYVNWIRRHITASGGSESGYPRLPLLAGLFLPGLFLLLVSCVLLAKCLLHPSADGTPFPAPD

SEQ ID NO: 15 - protein, 540 aa, Homo sapiens
MAKGEGAESGSAAGLLPTSILQSTERPAQVKKEPKKKKQQLSVCNKLCYALGGAPYQVTGCALGFFLQIYLLDVAQKDEEVVFCFSSFQVGPFSASIILFVGRAWDAITDPLVGLCISKSPWTCLGRLMPWIIFSTPLAVIAYFLIWFVPDFPHGQTYWYLLFYCLFETMVTCFHVPYSALTMFISTEQTERDSATAYRMTVEVLGTVLGTAIQGQIVGQADTPCFQDLNSSTVASQSANHTHGTTSHRETQKAYLLAAGVIVCIYIICAVILILGVREQREPYEAQQSEPIAYFRGLRLVMSHGPYIKLITGFLFTSLAFMLVEGNFVLFCTYTLGFRNEFQNLLLAIMLSATLTIPIWQWFLTRFGKKTAVYVGISSAVPFLILVALMESNLIITYAVAVAAGISVAAAFLLPWSMLPDVIDDFHLKQPHFHGTEPIFFSFYVFFTKFASGVSLGISTLSLDFAGYQTRGCSQPERVKFTLNMLVTMAPIVLILLGLLLFKMYPIDEERRRQNKKALQALRDEASSSGCSETDSTELA

SEQ ID NO: 16 - protein, 417 aa, Homo sapiens
MRLILPVGLIATTLAIAPVRFDREKVFRVKPQDEKQADIIKDLAKTNELDFWYPGATHHVAANMMVDFRVSEKESQAIQSALDQNKMHYEILIHDLQEEIEKQFDVKEDIPGRHSYAKYNNWEKIVAWTEKMMDKYPEMVSRIKIGSTVEDNPLYVLKIGEKNERRKAIFMDCGIHAREWVSPAFCQWFVYQATKTYGRNKIMTKLLDRMNFYILPVFNVDGYIWSWTKNRMWRKNRSKNQNSKCIGTDLNRNFNASWNSIPNTNDPCADNYRGSAPESEKETKAVTNFIRSHLNEIKVYITFHSYSQMLLFPYGYTSKLPPNHEDLAKVAKIGTDVLSTRYETRYIYGPIESTIYPISGSSLDWAYDLGIKHTFAFELRDKGKFGFLLPESRIKPTCRETMLAVKFIAKYILKHTS

SEQ ID NO: 17 - protein, 338 aa, Homo sapiens
MINSTSTQPPDESCSQNLLITQQIIPVLYCMVFIAGILLNGVSGWIFFYVPSSKSFIIYLKNIVIADFVMSLTFPFKILGDSGLGPWQLNVFVCRVSAVLFYVNMYVSIVFFGLISFDRYYKIVKPLWTSFIQSVSYSKLLSVIVWMLMLLLAVPNIILTNQSVREVTQIKCIELKSELGRKWHKASNYIFVAIFWIVFLLLIVFYTAITKKIFKSHLKSSRNSTSVKKKSSRNIFSIVFVFFVCFVPYHIARIPYTKSQTEAHYSCQSKEILRYMKEFTLLLSAANVCLDPIIYFFLCQPFREILCKKLHIPLKAQNDLDISRIKRGNTTLESTDTL

SEQ ID NO: 18 - protein, 852 aa, Homo sapiens
MAMRSGRHPSLLLLLVLLLWLLQVSIIDSVQQETDDLTKQTKEKIYQPLRRSKRRWVITTLELEEEDPGPFPKLIGELFNNMSYNMSLMYLISGPGVDEYPEIGLFSLEDHENGRIYVHRPVDREMTPSFTVYFDVVERSTGKIVDTSLIFNIRISDVNDHAPQFPEKEFNITVQENQSAGQPIFQMLAVDLDEENTPNSQVLYFLISQTPLLKESGFRVDRLSGEIRLSGCLDYETAPQFTLLIRARDCGEPSLSSTTTVHVDVQEGNNHRPAFTQENYKVQIPEGRASQGVLRLLVQDRDSPFTSAWRAKFNILHGNEEGHFDISTDPETNEGILNVIKPLDYETRPAQSLIIVVENEERLVFCERGKLQPPRKAAASATVSVQVTDANDPPAFHPQSFIVNKEEGARPGTLLGTFNAMDPDSQIRYELVHDPANWVSVDKNSGVVITVEPIDRESPHVNNSFYVIIIHAVDDGFPPQTATGTLMLFLSDINDNVPTLRPRSRYMEVCESAVHEPLHIEAEDPDLEPFSDPFTFELDNTWGNAEDTWKLGRNWGQSVELLTLRSLPRGNYLVPLFIGDKQGLSQKQTVHVRICPCASGLTCVELADAEVGLHVGALFPVCAAFVALAVALLFLLRCYFVLEPKRHGCSVSNDEGHQTLVMYNAESKGTSAQTWSDVEGQRPALLICTAAAGPTQGVKAYPDATMHRQLLAPVEGRMAETLNQSKERNRFSLSRGCIIPQGRATAGRGLPQDIYKEMMPRRLTQTGKRKHGALARTPSFKKVVYDHKEDEENKAGRKQRSHLFKVMQLRNEQGGVRVQSAHSPSPLNKKACFPGDYRGESAGGHNCRAVSG

SEQ ID NO: 19 - protein, 782 aa, Homo sapiens
MAPHRPAPALLCALSLALCALSLPVRAATASRGASQAGAPQGRVPEARPNSMVVEHPEFLKAGKEPGLQIWRVEKFDLVPVPTNLYGDFFTGDAYVILKTVQLRNGNLQYDLHYWLGNECSQDESGAAAIFTVQLDDYLNGRAVQHREVQGFESATFLGYFKSGLKYKKGGVASGFKHVVPNEVVVQRLFQVKGRRVVRATEVPVSWESFNNGDCFILDLGNNIHQWCGSNSNRYERLKATQVSKGIRDNERSGRARVHVSEEGTEPEAMLQVLGPKPALPAGTEDTAKEDAANRKLAKLYKVSNGAGTMSVSLVADENPFAQGALKSEDCFILDHGKDGKIFVWKGKQANTEERKAALKTASDFITKMDYPKQTQVSVLPEGGETPLFKQFFKNWRDPDQTDGLGLSYLSSHIANVERVPFDAATLHTSTAMAAQHGMDDDGTGQKQIWRIEGSNKVPVDPATYGQFYGGDSYIILYNYRHGGRQGQIIYNWQGAQSTQDEVAASAILTAQLDEELGGTPVQSRVVQGKEPAHLMSLFGGKPMIIYKGGTSREGGQTAPASTRLFQVRANSAGATRAVEVLPKAGALNSNDAFVLKTPSAAYLWVGTGASEAEKTGAQELLRVLRAQPVQVAEGSEPDGFWEALGGKAAYRTSPRLKDKKMDAHPPRLFACSNKIGRFVIEEVPGELMQEDLATDDVMLLDTWDQVFVWVGKDSQEEEKTEALTSAKRYIETDPANRDRRTPITVVKQGFEPPSFVGWFLGWDDDYWSVDPLDRAMAELAA

SEQ ID NO: 20 - protein, 164 aa, Homo sapiens
MGDLPGLVRLSIALRIQPNDGPVFYKVDGQRFGQNRTIKLLTGSSYKVEVKIKPSTLQVENISIGGVLVPLELKSKEPDGDRVVYTGTYDTEGVTPTKSGERQPIQITMPFTDIGTFETVWQVKFYNYHKRDHCQWGSPFSVIEYECKPNETRSLMWVNKESFL

SEQ ID NO: 21 - protein, 295 aa, Homo sapiens
MYRISQLMSTPVASSSRLEREYAGELSPTCIFPSFTCDSLDGYHSFECGSIDPLTGSHYTCRRSPRLLTNGYYIWTEDSFLCDKDGNITLNPSQTSVMYKENLVRIFRKKKRICHSFSSLFNLSTSKSWLHGSIFGDINSSPSEDNWLKGTRRLDTDHCNGNADDLDCSSLTDDWESGKMNAESVITSSSSHIISQPPGGNSHSLSLQSQLTASERFQENSSDHSETRLLQEVFFQAILLAVCLIISACARWFMGEILASVFTCSLMITVAYVKSLFLSLASYFKTTACARFVKI

SEQ ID NO: 22 - protein, 198 aa, Homo sapiens
MDAILNYRSEDTEDYYTLLGCDELSSVEQILAEFKVRALECHPDKHPENPKAVETFQKLQKAKEILTNEESRARYDHWRRSQMSMPFQQWEALNDSVKTSMHWVVRGKKDLMLEESDKTHTTKMENEECNEQRERKKEELASTAEKTEQKEPKPLEKSVSPQNSDSSGFADVNGWHLRFRWSKDAPSELLRKFRNYEI

SEQ ID NO: 23 - protein, 159 aa, Homo sapiens
MSRRNCWICKMCRDESKRPPSNLTLEEVLQWAQSFENLMATKYGPVVYAAYLKMEHSDENIQFWMACETYKKIASRWSRISRAKKLYKIYIQPQSPREINIDSSTRETIIRNIQEPTETCFEEAQKIVYMHMERDSYPRFLKSEMYQKLLKTMQSNNSF

SEQ ID NO: 24 - protein, 514 aa, Homo sapiens
MALSELALVRWLQESRRSRKLILFIVFLALLLDNMLLTVVVPIIPSYLYSIKHEKNATEIQTARPVHTASISDSFQSIFSYYDNSTMVTGNATRDLTLHQTATQHMVTNASAVPSDCPSEDKDLLNENVQVGLLFASKATVQLITNPFIGLLTNRIGYPIPIFAGFCIMFVSTIMFAFSSSYAFLLIARSLQGIGSSCSSVAGMGMLASVYTDDEERGNVMGIALGGLAMGVLVGPPFGSVLYEFVGKTAPFLVLAALVLLDGAIQLFVLQPSRVQPESQKGTPLTTLLKDPYILIAAGSICFANMGIAMLEPALPIWMMETMCSRKWQLGVAFLPASISYLIGTNIFGILAHKMGRWLCALLGMIIVGVSILCIPFAKNIYGLIAPNFGVGFAIGMVDSSMMPIMGYLVDLRHVSVYGSVYAIADVAFCMGYAIGPSAGGAIAKAIGFPWLMTIIGIIDILFAPLCFFLRSPPAKEEKMAILMDHNCPIKTKMYTQNNIQSYPIGEDEESESD

SEQ ID NO: 25 - protein, 397 aa, Homo sapiens
MDSLATSINQFALELSKKLAESAQGKNIFFSSWSISTSLTIVYLGAKGTTAAQMAQVLQFNRDQGVKCDPESEKKRKMEFNLSNSEEIHSDFQTLISEILKPNDDYLLKTANAIYGEKTYAFHNKYLEDMKTYFGAEPQPVNFVEASDQIRKDINSWVERQTEGKIQNLLPDDSVDSTTRMILVNALYFKGIWEHQFLVQNTTEKPFRINETTSKPVQMMFMKKKLHIFHIEKPKAVGLQLYYKSRDLSLLILLPEDINGLEQLEKAITYEKLNEWTSADMMELYEVQLHLPKFKLEDSYDLKSTLSSMGMSDAFSQSKADFSGMSSARNLFLSNVFHKAFVEINEQGTEAAAGSGSEIDIRIRVPSIEFNANHPFLFFIRHNKTNTILFYGRLCSP

SEQ ID NO: 26 - protein, 729 aa, Homo sapiens
MDDLTLLDLLECPVCFEKLDVTAKVLPCQHTFCKPCLQRVFKAHKELRCPECRTPVFSNIEALPANLLLVRLLDGVRSGQSSGRGGSFRRPGTMTLQDGRKSRTNPRRLQASPFRLVPNVRIHMDGVPRAKALCNYRGQNPGDLRFNKGDIILLRRQLDENWYQGEINGISGNFPASSVEVIKQLPQPPPLCRALYNFDLRGKDKSENQDCLTFLKDDIITVISRVDENWAEGKLGDKVGIFPILFVEPNLTARHLLEKNKGRQSSCTKNLSLVSSSSRGNTSTLRRGPGSRRKVPGQFSITTALNTLNRMVHSPSGRHMVEISTPVLISSSNPSVITQPMEKADVPSSCVGQVSTYHPAPVSPGHSTAVVSLPGSQQHLSANMFVALHSYSAHGPDELDLQKGEGVRVLGKCQDGWLRGVSLVTGRVGIFPNNYVIPIFRKTSSFPDSRSPGLYTTWTLSTSSVSSQGSISEGDPRQSRPFKSVFVPTAIVNPVRSTAGPGTLGQGSLRKGRSSMRKNGSLQRPLQSGIPTLVVGSLRRSPTMVLRPQQFQFYQPQGIPSSPSAVVVEMGSKPALTGEPALTCISRGSEARIHSAASSLIMEDKEIPIKSEPLPKPPASAPPSILVKPENSRNGIEKQVKTVRFQNYSPPPTKHYTSHPTSGKPEQPATLKASQPEAASLGPEMTVLFAHRSGCHSGQQTDLRRKSALAKATTLVSTASGTQTVFPSK

SEQ ID NO: 27 - protein, 240 aa, Homo sapiens
MDTESNRRANLALPQEPSSVPAFEVLEISPQEVSSGRLLKSASSPPLHTWLTVLKKEQEFLGVTQILTAMICLCFGTVVCSVLDISHIEGDIFSSFKAGYPFWGAIFFSISGMLSIISERRNATYLVRGSLGANTASSIAGGTGITILIINLKKSLAYIHIHSCQKFFETKCFMASFSTEIVVMMLFLTILGLGSAVSLTICGAGEELKGNKVPEDRVYEELNIYSATYSELEDPGEMSP

SEQ ID NO: 28 - protein, 521 aa, Homo sapiens
MASNSLFSTVTPCQQNFFWDPSTSRRFSPPSSSLQPGKMSDVSPVVAAQQQQQQQQQQQQQQQQQQQQQQQEAAAAAAAAAAAAAAAAAVPRLRPPHDNRTMVEIIADHPAELVRTDSPNFLCSVLPSHWRCNKTLPVAFKVVALGEVPDGTVVTVMAGNDENYSAELRNASAVMKNQVARFNDLRFVGRSGRGKSFTLTITVFTNPPQVATYHRAIKVTVDGPREPRRHRQKLDDSKPSLFSDRLSDLGRIPHPSMRVGVPPQNPRPSLNSAPSPFNPQGQSQITDPRQAQSSPPWSYDQSYPSYLSQMTSPSIHSTTPLSSTRGTGLPAITDVPRRISDDDTATSDFCLWPSTLSKKSQAGASELGPFSDPRQFPSISSLTESRFSNPRMHYPATFTYTPPVTSGMSLGMSATTHYHTYLPPPYPGSSQSQSGPFQTSSTPYLYYGTSSGSYQFPMVPGGDRSPSRMLPPCTTTSNGSTLLNPNLPNQNDGVDADGSHSSSPTVLNSSGRMDESVWRPY

SEQ ID NO: 29 - protein, 599 aa, Homo sapiens [sequence omitted]

SEQ ID NO: 30 - protein, 662 aa, Homo sapiens
MGLYRIRVSTGASLYAGSNNQVQLWLVGQHGEAALGKRLWPARGKETELKVEVPEYLGPLLFVKLRKRHLLKDDAWFCNWISVQGPGAGDEVRFPCYRWVEGNGVLSLPEGTGRTVGEDPQGLFQKHREEELEERRKLYRWGNWKDGLILNMAGAKLYDLPVDERFLEDKRVDFEVSLAKGLADLAIKDSLNVLTCWKDLDDFNRIFWCGQSKLAERVRDSWKEDALFGYQFLNGANPVVLRRSAHLPARLVFPPGMEELQAQLEKELEGGTLFEADFSLLDGIKANVILCSQQHLAAPLVMLKLQPDGKLLPMVIQLQLPRTGSPPPPLFLPTDPPMAWLLAKCWVRSSDFQLHELQSHLLRGHLMAEVIVVATMRCLPSIHPIFKLIIPHLRYTLEINVRARTGLVSDMGIFDQIMSTGGGGHVQLLKQAGAFLTYSSFCPPDDLADRGLLGVKSSFYAQDALRLWEIIYRYVEGIVSLHYKTDVAVKDDPELQTWCREITEIGLQGAQDRGFPVSLQARDQVCHFVTMCIFTCTGQHASVHLGQLDWYSWVPNAPCTMRLPPPTTKDATLETVMATLPNFHQASLQMSITWQLGRRQPVMVAVGQHEEEYFSGPEPKAVLKKFREELAALDKEIEIRNAKLDMPYEYLRPSVVENSVAI

SEQ ID NO: 31 - DNA, 530 bp, Homo sapiens
  modified_base (484)..(484): a, c, g, t, unknown or other
aaagaatctgacatcatgacaacaaatggtgtaattcatgttgtagataaactcctctatccagcagacacacctgttggaaatgatcaactgctggaaatacttaataaattaatcaaatacatccaaattaagtttgttcgtggtagcaccttcaaagaaatccccgtgactgtctatagacccacactaacaaaagtcaaaattgaaggtgaacctgaattcagactgattaaagaaggtgaaacaataactgaagtgatccatggagagccaattattaaaaaatacaccaaaatcattgatggagtgcctgtggaaataactgaaaaagagacacgagaagaacgaatcattacaggtcctgaaataaaatacactaggatttctactggaggtggagaaacagaagaaactctgaagaaattgttacaagaagaagacacacccgtgaggaagttgcaagccaacaaaaaagttcaanggatctagaagacgattaagggaaggtcgttctcagtgaaaatcca

SEQ ID NO: 32 - DNA, 413 bp, Homo sapiens
aaattgtggagttagcctcctgtggagttagcctcctgtggtaaaggaattgaagaaaatataacaccttacaccctttttcatcttgacattaaaagttctggctaactttggaatccattagagaaaaatccttgtcaccagattcattacaattcaaatcgaagagttgtgaactgttatcccattgaaaagaccgagccttgtatgtatgttatggatacataaaatgcacgcaagccattatctctccatgggaagctaagttataaaaataggtgcttggtgtacaaaactttttatatcaaaaggctttgcacatttctatatgagtgggtttactggtaaattatgttattttttacaactaattttgtactctcagaatgtttgtcatatgcttcttgcaatgc

SEQ ID NO: 33 - DNA, 493 bp, Homo sapiens
gcgagtacaacaaggccaccgaagatgagtactacagacgcccgctgcaggtgctgcgagccagggagcagacctttgggggggtgaattacttcttcgacgtagaggtgggccgcaccatatgtaccaagtcccagcccaacttggacacctgtgccttccatgaacagccagaactgcagaagaaacagttatgctctttcgagatctacgaagttccctgggaggacagaatgtccctggtgaattccaggtgtcaagaagcctaggggtctgtgccaggccagtcacaccgaccaccacccactcccaccccctgtagtgctcccacccctggactggtggcccccaccctgcgggaggcctccccatgtgcctgtgccaagagacagacagagaaggctgcaggagtcctttgttgctcagcagggcgctctgccctccctccttccttcttgcttctaatagacctggtacatggtacacacacccc

SEQ ID NO: 34 - DNA, 365 bp, Homo sapiens
ggaggataggataatcccgggtggcatctataacgcagacctcaatgatgagtgggtacagcgtgcccttcacttcgccatcagcgagtataacaaggccaccaaagatgactactacagacgtccgctgcgggtactaagagccaggcaacagaccgttgggggggtgaattacttcttcgacgtagaggtgggccgaaccatatgtaccaagtcccagcccaacttggacacctgtgccttccatgaacagccagaactgcagaagaaacagttgtgctctttcgagatctacgaagttccctgggagaacagaaggtccctggtgaaatccaggtgtcaagaatcctagggatctgtgccag

SEQ ID NO: 35 - DNA, 410 bp, Homo sapiens
gagaagggcctgatttgcagcatcatgatgggcctctccttggcctctgctgtgctcctggcctccctcctgagtctccaccttggaactgccacacgtgggagtgacatatccaagacctgctgcttccaatacagccacaagccccttccctggacctgggtgcgaagctatgaattcaccagtaacagctgctcccagcgggctgtgatattcactaccaaaagaggcaagaaagtctgtacccatccaaggaaaaaatgggtgcaaaaatacatttctttactgaaaactccgaaacaattgtgactcagctgaattttcatccgaggacgcttggaccccgctcttggctctgcagccctctggggagcctgcggaatcttttctgaaggctacatggacccgct

SEQ ID NO: 36 - DNA, 489 bp, Homo sapiens
ggccaaatcaccgacctgaaggcggaaattcacgggggcagtctcattaatctgacttggacagctcctggggatgattatgaccatggaacagctcacaagtatatcattcgaataagtacaagtattcttgatctcagagacaagttcaatgaatctcttcaagtgaatactactgctctcatcccaaaggaagccaactctgaggaagtctttttgtttaaaccagaaaacattacttttgaaaatggcacagatcttttcattgctattcaggctgttgataaggtcgatctgaaatcagaaatatccaacattgcacgagtatctttgtttattcctccacagactccgccagagacacctagtcctgatgaaacgtctgctccttgtcctaatattcatatcaacagcaccattcctggcattcacattttaaaaattatgtggaagtggataggagaactgcagctgtcaatagcctagggc

SEQ ID NO: 37 - DNA, 324 bp, Homo sapiens
gagcccccaggaggaggacaggataatcgagggtggcatctatgatgcagacctcaatgatgagcgggtacagcgtgcccttcactttgtcatcagcgagtataacaaggccactgaagatgagtactacagacgcctgctgcgggtgctacgagccagggagcagatcgtgggcggggtgaattacttcttcgacatagaggtgggccgaaccatatgtaccaagtcccagcccaacttggacacctgtgccttccatgaacagccagaactgcagaagaaacagttgtgctctttccagatctacgaagttccctgggagga

SEQ ID NO: 38 - DNA, 310 bp, Homo sapiens
aagactttactttcaccataccagatgtagaggactcaagtcagagaccagatcagggaccccagagacctcctcctgaaggactcctacctagaccccctggtgatagtggtaaccaagatgatggtcctcagcagagaccaccaaaaccaggaggccatcaccgccatcctcccccacctccttttcaaaatcagcaacgaccaccccaacgaggacaccgtcaactctctctaccccgatttccttctgtcagcctgcaggaagcatcatcattcttccggagggacagaccagcaagacatcccca

SEQ ID NO: 39 - DNA, 465 bp, Homo sapiens
ttcctcaccctaaaactaagcgtgctgcttctgcaaaagatttttgtagatgagctgtgtgcctcagaattgctatttcaaattgccaaaaatttagagatgttttctacatatttctgctcttctgaacaacttctgctacccactaaataaaaacacagaaataattagacaattgtctattataacatgacaaccctattaatcatttggtcttctaaaatgggatcatgcccatttagattttccttactatcagtttatttttataacattaacttttactttgttatttattattttatataatggtgagtttttaaattattgctcactgcctatttaatgtagctaataaagttatagaagcagatgatctgttaatttcctatctaataaatgcctttaattgttctcataatgaagaataagtaggtaccctccatgcccttctgtaataaatat

SEQ ID NO: 40 - DNA, 323 bp, Homo sapiens
agaagactctgacctgtactcttgaatacaagtttctgataccactgcactgtctgagaatttccaaaactttaatgaactaactgacagcttcatgaaactgtccaccaagatcaagcagagaaaataattaatttcatgggactaaatgaactaatgaggattgctgattctttaaatgtcttgtttcccagatttcaggaaactttttttcttttaagctatccactcttacagcaatttgataaaatatacttttgtgaacaaaaattgagacatttacattttctccctatgtggtcgctccagacttgggaaactat

SEQ ID NO: 41 - DNA, 500 bp, Homo sapiens
tcatcgggcctggcacaggcatcgcgcccttccgcagtttctggcagcaacggctccatgactcccagcacaagggagtgcggggaggccgcatgaccttggtgtttgggtgccgccgcccagatgaggaccacatctaccaggaggagatgctggagatggcccagaagggggtgctgcatgcggtgcacacagcctattcccgcctgcctggcaagcccaaggtctatgttcaggacatcctgcggcagcagctggccagcgaggtgctccgtgtgctccacaaggagccaggccacctctatgtttgcggggatgtgcgcatggcccgggacgtggcccacaccctgaagcagctggtggctgccaagctgaaattgaatgaggagcaggtcgaggactatttctttcagctcaagagccagaagcgctatcacgaagatatctttggtgctgtatttccttacgaggcgaagaaggacagggtggcggtgcagccc

SEQ ID NO: 42 - DNA, 363 bp, Homo sapiens
gtcgatttacacttacctcggttcaaaatggaagagagctatgacctcaaggacacgttgagaaccatgggaatggtgaatatcttcaatggggatgcagacctctcaggcatgacctggagccacggtctctcagtatctaaagtcctacacaaggcctttgtggaggtcactgaggagggagtggaagctgcagctgccaccgctgtagtagtagtcgaattatcatctccttcaactaatgaagagttctgttgtaatcaccctttcctattcttcataaggcaaaataagaccaacagcatcctcttctatggcagattctcatccccatagatgcaattagtctgtcactccatttag

SEQ ID NO: 43 - DNA, 508 bp, Homo sapiens
gatacgacactggttcttgtgaacgcaatctatttcaaagggcagtgggagaataaatttaaaaaagaaaacactaaagaggaaaaattttggccaaacaaggatgtacaggccaaggtcctggaaataccatacaaaggcaaagatctaagcatgattgtgctgctgccaaatgaaatcgatggtctgcagaagcttgaagagaaactcactgctgagaaattgatggaatggacaagtttgcagaatatgagagagacatgtgtcgatttacacttacctcggttcaaaatggaagagagctatgacctcaaggacacgttgagaaccatgggaatggtgaatatcttcaatggggatgcagacctctcaggcatgacctggagccacggtctctcagtatctaaagtcctacacaaggcctttgtggaggtcactgaggagggagtggaagctgcagctgccaccgctgtagtagtagtcgaattatcatctccttcaactaatg

SEQ ID NO: 44 - DNA, 493 bp, Homo sapiens; identical to SEQ ID NO: 33

SEQ ID NO: 45 - DNA, 447 bp, Homo sapiens
ccacctcctccaggaaagccagaaagaccacccccacaaggaggtaaccagtcccaaggtcccccacctcatccaggaaagccagaaggaccacccccacaggaaggaaacaagtcccgaagtgcccgatctcctccaggaaagccacaaggaccaccccaacaagaaggcaacaagcctcaaggtcccccacctcctggaaagccacaaggcccacccccagcaggaggcaatccccagcagcctcaggcacctcctgctggaaagccccaggggccacctccacctcctcaagggggcaggccacccagacctgcccagggacaacagcctccccagtaatctaggattcaatgacaggaagtgaataagaagatatcagtgaattcaaataattcaattgctacaaatgccgtgacattggaacaaggtcatcatagctctaac

SEQ ID NO: 46 - DNA, 304 bp, Homo sapiens
tgacgcaaaataccaccttggcgcctacacgggagacgacgtccgcatcatccgtgacgacatgctgtgtgccgggaacagccagagggactcctgcaagggcgactctggagggcccctggtgtgcaaggtgaatggcacctggctacaggcgggcgtggtcagctgggacgagggctgtgcccagcccaaccggcctggcatctacacccgtgtcacctactacttggactggatccaccactatgtccccaaaaagccgtgagtcaggcctgggtgtgccacctgggtcactggaggacca

SEQ ID NO: 47 - DNA, 358 bp, Homo sapiens
ccgccatttcctctgaagcaggtgaaggtccccataatggaaaaccacatttgtgacgcaaaataccaccttggcgcctacacgggagacgacgtccgcatcgtccgtgacgacatgctgtgtgccgggaacacccggagggactcatgccagggcgactccggagggcccctggtgtgcaaggtgaatggcacctggctgcaggcgggcgtggtcagctggggcgagggctgtgcccagcccaaccggcctggcatctacacccgtgtcacctactacttggactggatccaccactatgtccccaaaaagccgtgagtcaggcctgggttggccacctgggtcactggaggaccaa

SEQ ID NO: 48 - DNA, 542 bp, Homo sapiens
tgacgcaaaataccaccttggcgcctacacgggagacgacgtccgcatcgtccgtgacgacatgctgtgtgccgggaacacccggagggactcatgccagggcgactccggagggcccctggtgtgcaaggtgaatggcacctggctgcaggcgggcgtggtcagctggggcgagggctgtgcccagcccaaccggcctggcatctacacccgtgtcacctactacttggactggatccaccactatgtccccaaaaagccgtgagtcaggcctgggttggccacctgggtcactggaggaccaacccctgctgtccaaaacaccactgcttcctacccaggtggcgactgccccccacaccttccctgccccgtcctgagtgccccttcctgtcctaagccccctgctctcttctgagccccttcccctgtcctgaggacccttccctatcctgagcccccttccctgtcctaagcctgacgcctgcaccgggccctccagccctcccctgcccagatagctggtggtgggcgctaatcct

SEQ ID NO: 49 - DNA, 536 bp, Homo sapiens
tgacgcaaaataccaccttggcgcctacacgggagacgacgtccgcatcgtccgtgacgacatgctgtgtgccgggaacacccggagggactcatgccagggcgactccggagggcccctggtgtgcaaggtgaatggcacctggctgcaggcgggcgtggtcagctggggcgagggctgtgcccagcccaaccggcctggcatctacacccgtgtcacctactacttggactggatccaccactatgtccccaaaaagccgtgagtcaggcctgggttggccacctgggtcactggaggaccaacccctgctgtccaaaacaccactgcttcctacccaggtggcgactgccccccacaccttccctgccccgtcctgagtgccccttcctgtcctaagccccctgctctcttctgagccccttcccctgtcctgaggacccttccccatcctgagcccccttccctgtcctaagcctgacgcctgcaccgggccctccggccctcccctgcccaggcagctggtggtgggcgct

SEQ ID NO: 50 - DNA, 538 bp, Homo sapiens
ccggtcagcaggatcatcgtgcacccacagttctacatcatccagactggagcggatatcgccctgctggagctggaggagcccgtgaacatctccagccgcgtccacacggtcatgctgccccctgcctcggagaccttccccccggggatgccgtgctgggtcactggctggggcgatgtggacaatgatgagcccctcccaccgccatttcccctgaagcaggtgaaggtccccataatggaaaaccacatttgtgacgcaaaataccaccttggcgcctacacgggagacgacgtccgcatcatccgtgacgacatgctgtgtgccgggaacacccggagggactcatgccagggcgactctggagggcccctggtgtgcaaggtgaatggcacctggctacaggcgggcgtggtcagctgggacgagggctgtgcccagcccaaccggcctggcatctacacccgtgtcacctactacttggactggatccaccactatgtccccaaaaagccgtgagtcaggcctggggtgt

SEQ ID NO: 51 - DNA, 366 bp, Homo sapiens
ccggtcagcaggatcatcgtgcacccacagttctacaccgcccagatcggagcggacatcgccctgctggagctggaggagccggtgaacgtctccagccacgtccacacggtcaccctgccccctgcctcagagaccttccccccggggatgccgtgctgggtcactggctggggcgatgtggacaatgatgagcgcctcccaccgccatttcctctgaagcaggtgaaggtccccataatggaaaaccacatttgtgacgcaaaataccaccttggcgcctacacgggagacgacgtccgcatcgtccgtgacgacatgctgtgtgccgggaacacccggagggactcatgccaggtggcgact

SEQ ID NO: 52 - DNA, 496 bp, Homo sapiens
  modified_base (150)..(151): a, c, g, t, unknown or other
ccggtcagcaggatcatcgtgcacccacagttctacatcatccagactggagcggatatcgccctgctggagctggaggagcccgtgaacatctccagccgcgtccacacggtcatgctgccccctgcctcggagaccttccccccgggnntgccgtgctgggtcactggctggggcgatgtggacaatgatgagcccctcccaccgccatttcccctgaagcaggtgaaggtccccataatggaaaaccacatttgtgacgcaaaataccaccttggcgcctacacgggagacgacgtccgcatcatccgtgacgacatgctgtgtgccgggaacacccggagngnntcatgccagggcgactcnggagggcccctggtgtgcaaggtgaatggcacctggctncaggcgggcgtggtcagctgggncgagggctgtgcccagcccaaccggcctggcatctacacccgtgtcacctactacttggactggatcc

SEQ ID NO: 53 - DNA, 408 bp, Homo sapiens
gtcgtcacggacgatgcggacgtcgtctcccgtgtaggcgccaaggtggtattttgcgtcacaaatgtggttttccattatggggaccttcacctgcttcagaggaaatggcggtgggaggcgctcatcattgtccacatcgccccagccagtgacccagcacggcatccccggggggaaggtctctgaggcagggggcagggtgaccgtgtggacgtggctggagacgttcaccggctcctccagctccagcagggcgatgtccgctccgatctgggcggtgtagaactgtgggtgcacgatgatcctgctgaccggcagcagctggtcctggtagtagaggtgctgctcccgcagttgcaccggtcccacgcagtgcgctgcggtcagcacccactgggggtggat

SEQ ID NO: 54 - DNA, 520 bp, Homo sapiens
ggtgaaagtctccgtggtggacacagagacctgccgccgggactatcccggccccgggggcagcatccttcagcccgacatgctgtgtgcccggggccccggggatgcctgccaggacgactccggggggcctctggtctgccaggtgaacggtgcctgggtgcaggctggcattgtgagctggggtgagggctgcggccgccccaacaggccgggagtctacactcgtgtccctgcctacgtgaactggatccgccgccacatcacagcatcagggggctcagagtctgggtaccccaggctccccctcctggctggcttattcctccccggcctcttccttctgctagtctcctgtgtcctgctggccaagtgcctgctgcacccatctgcggatggtactcccttccccgcccctgactgatggcaggaatccaagtgcatttcttaaataagttactatttattccgctccgccccctccctctcccttgagaagctgagtcttctgcatcagatt

SEQ ID NO: 55 - DNA, 508 bp, Homo sapiens
tgccacaaggtcgctgcttatgagggcgcaaacttcttggcttgtgctcggacccttttctcgtactccactctgttttggcagtaaatcgtgtaggcctctgcttgagctgggtcttggatatttggttcatttagaagttcctgtattcctaataggatctgtttgattgtgatggctggcctccagtccttgtcctcctctaagatggacaggcacactgtccccgaagggtacacattcgggtgaaataatggtggttcgaatttacattttggtggcgaagatggataatcatctttgaaaagcatccgtagtttaaacaagcctccttcccacggagtccctttctttcctggaatggcgcactcccagttcatgaggttcatcgtgccatcgggattttttgttgggacagccacgaaaccaaatgggtggtctttcctccatgctttcctctcctgggcgagtctgctgagggcgatccccgacatgttcaaagtccctc

SEQ ID NO: 56 - DNA, 510 bp, Homo sapiens
agagactttcagggcatacgtgggggccttggccttcctcactcgctcgatggcctcagtgtgctcctcaaggctggtgccaaacacctgctggagatagctgagcagggcctcctcgtcgtccacctggtcagggcccatggtacccgcgcggtaaagcaccgtgtacagggcctcctcgtagagcatctccacctcctctggggccagggctctcaggccgaggctgggatccacaggctccgggggtgctggcgagccactgcgcagggggacctcgaggcacggcaagccctgtctgccttcccccttcttcagcatgaggcgcatgtgggcaaagaactccacgccatccccgggtttccaggcccccgtggcaggctcctgcgggtcggcgctggcactccctgggtcctgctcagtcctgcggcggaaggacgggcacacctgcacctgcctgagcacgctgctcttaatgtccagcaaggtcgacatggcgggtgaccgtgg

SEQ ID NO: 57 - DNA, 407 bp, Homo sapiens
tgctgctcttcaaaatgtaccccattgatgaggagaggcggcggcagaataagaaggccctgcaggcactgagggacgaggccagcagctctggctgctcagaaacagactccacagagctggctagcatcctctagggcccgccacgttgcccgaagccaccatgcagaaggccacagaagggatcaggacctgtctgccggcttgctgagcagctggactgcaggtgctaggaagggaactgaagactcaaggaggtggcccaggacacttgctgtgctcactgtggggccggctgctctgtggcctcctgcctcccctctgcctgcctgtggggccaagccctggggctgccactgtgaatatgccaaggactgatcgggcctagcccggaacactaatgtaga

SEQ ID NO: 58 - DNA, 565 bp, Homo sapiens
tatgaaacccgctacatctatggcccaatagaatcaacaatttacccgatatcaggttcttctttagactgggcttatgacctgggcatcaaacacacatttgcctttgagctccgagataaaggcaaatttggttttctccttccagaatcccggataaagccaacgtgcagagagaccatgctagctgtcaaatttattgccaagtatatcctcaagcatacttcctaaagaactgccctctgtttggaataagccaattaatccttttttgtgcctttcatcagaaagtcaatcttcagttatccccaaatgcagcttctatttcacctgaatccttctcttgctcatttaagtcccatgttactgctgtttgcttttacttactttcagtagcaccataacgaagtagctttaagtgaaaccttttaactacctttctttgctccaagtgaagtttggacccagcagaaagcattattttgaaaggtgatatacagtggggcacagaaaacaaatgaaaaccctcagtttctcacagattttcaccatgtggcttcatcaa

SEQ ID NO: 59 - DNA, 459 bp, Homo sapiens
tgagcctggggttctggtgttagaatatttttaagtaggctttactgagagaaactaaatattggcatacgttatcagcaacttcccctgttcaatagtatgggaaaaataagatgactgggaaaaagacacacccacaccgtagaacatatattaatctactggcgaatgggaaaggagaccattttcttagaaagcaaataaacttgatttttttaaatctaaaatttacattaatgagtgcaaaataacacataaaatgaaaattcacacatcacatttttctggaaaacagacggattttacttctggagacatggcatacggttactgacttatgagctaccaaaactaaattctttctctgctattaactggctagaagacattcatctatttttcaaatgttctttcaaaacatttttataagtaatgtttgtatctatttcatgctttact

SEQ ID NO: 60 - DNA, 430 bp, Homo sapiens
  modified_base (343)..(345): a, c, g, t, unknown or other
gggaatcactattcagggatttttcccctttgctcttcttttccctccttaaaagaaaaattaccttctagtcctaggatgaggacacactattagtttgaattaaatgctttgatattctcagatcagccatcttgaaccaaagcaaaaccacaagttacactttcttaaaatttgatttgtcatattttctagagaaacttgaatttaattgtgttattcttagcttccactggcagcctagctttgagggtaaatgaaaatataacccatagattacccagccacttgggaacagcaggtaatactgaagaaaaataaaaatagattttgaaaacgttannnanannnntatgattatgattctgttccatttaagggaaaacttaggtaaatagagaaattttttctataacattgtgtagtcagt

SEQ ID NO: 61 - DNA, 358 bp, Homo sapiens
tgcttctggacacctgggaccaggtctttgtctgggttggaaaggattctcaagaagaagaaaagacagaagccttgacttctgctaagcggtacatcgagacggacccagccaatcgggatcggcggacgcccatcaccgtggtgaagcaaggctttgagcctccctcctttgtgggctggttccttggctgggatgatgattactggtctgtggaccccttggacagggccatggctgagctggctgcctgaggaggggcagggcccacccatgtcaccggtcagtgccttttggaactgtccttccctcaaagaggccttagagcgagcagagcagctctgctatgagtgtgtgt

SEQ ID NO: 62 - DNA, 506 bp, Homo sapiens
  modified_base (86)..(86): a, c, g, t, unknown or other
acttttgaccacttgtgactggagttcagtggccctggcaggcttgtcctgctcttgaccattccactgactaactttggtgtttngtttccaagttaagtgattcctcctttttttngttcaatgttaaatttaaaaataacaatgtgtatgggtcctcccatgtgtaatatggtaacatgtaacttgcagtgtttgccagctttcaaagcaggctttgtgaaaatgtaatacaaacagcagtgaatgggactcaaatgttgtgcttcctataaacagctccgctctttcagggaaggatggtaacaaactagaaggacaaatatgtacgtatttataacgtattaaaactcttttaagtagcttaaggtattgtgcaatggcctagcctagtagaaatgggggaaaagcattgctgtggaccattgttaaagtgacaggagttgtagggttacccctttgacaagcttccatagtcttcagacacgcacattgatggcatccct

SEQ ID NO: 63 - DNA, 496 bp, Homo sapiens
  modified_base (60)..(60): a, c, g, t, unknown or other
ctaactaataccaacctgacaacttgaataacaataaatgcaatttgtacataaaatatnatgctgcaaaagttngtcattcacctcagtggagtgacttgatattaggtggtnaccgtagatgatggttnatatganaantggacaggaaagaagcantttctgaaagttatantcttttgaaccacgttctaaaccaagtntttnatcttcttggggctcgtaattacctttcactttaatgtcacttaaagatataacacagaaaaatgccttgagggcaaaatataggcaaaacaccaatgcgctttcaaatgcatgaaaatggtgcagttgtacccttgagccttgactcaagggctgtagatgttccctttccaccccccacacttggtgcgtgttcacaaagcaaatatggcctgtaattcaaatttgttctatgtgatactctctgagtaaaaactcatacatgcagaaaattgtctttgctcgaaat

SEQ ID NO: 64 - DNA, 560 bp, Homo sapiens
cccaagcccctagagaagtcagtctccccgcaaaattcagattcttcaggttttgcagatgtgaatggttggcaccttcgtttccgctggtccaaggatgctccctcagaactcctgaggaagttcagaaactatgaaatatgaaatatctctgcttcaaaaaatgaggaagagcaagactgtcccctatgctgccaacatgcagtctttgtttatgtcttaaaaatgtcatgtttatgtcatgtctgtgaattgctgagtactaattgattcctccatccttgaatcagttctcataatgctttttaaataagaaaaattcagaagatgaatttcttccaatatttgaataaattaaagctcttagatacagagtagattgtattatatgctttttcctattaatactacttatagaaatccattaaaaagcaatctctgtacagtgtatttaaatatttcattgacatactgtgatctctattagtgatggatgtacaaaaaatgttttcttacccttgacttacaatgaaatgtgaaattacttgtctgaaccccgt

SEQ ID NO: 65 - DNA, 512 bp, Homo sapiens
  modified_base (443)..(443): a, c, g, t, unknown or other
acagcaagcctatgtagttcaattaatatataaggaaaaggaaggtctttcttcatgatacaagcattataaagtttttactgtagtagtcaattaatggatatttccttgttaataaaattttgtgtcataatttacaaattagttctttaaaaattgttgttatatgaattgtgtttctagcatgaatgttctatagagtactctaaataacttgaatttatagacaaatgctactcacagtacaatcaattgtattataccatgagaaaatcaaaaaggtgttcttcagagacattttatctataaaattttcctactattatgttcattaacaaacttctttatcacatgtatcttctacgtgtaaaacatttctgatgattttttaacaaaaaatatatgaatttcttcatttgctcttgcatctacattgctataanggatataaaatgtggtttctatattttgagatgttttttccttacaatgtgaactcatcgtgatcttgg

SEQ ID NO: 66 - DNA, 551 bp, Homo sapiens
  modified_base (114)..(114): a, c, g, t, unknown or other
ctgctactttggaagatggctctggaggaaactctcatatggctaaaaaggcaggctagtttcttacttctacaggggtagagccttaaaaaagaacgtgctacaaattggttntcttnnagggttncnggttctccctgcccccaatnccnatatactttantgcnnttttatttttgcctttacggnctctgtgtctttctgcaagaaggcctggcaaaggtatgcctgctgttggtcccntcgggataagataaaatataaataaaaccttcagaactgttttggagcaaaagatagcttgtacttggggaaaaaaattctaagttcttttatatgactaatattcttggttagcaagactggaaagaggtgtttttttaaaatgtacataccagaacaaagaacatacagctctctgaacatttattttttgaacagaggtggtttttatgtttggacctggtaatacagatacaaaaactttaatgaggtagcaatgaatattcaactgtttgactgctaagtgtatctgtccatattttagcaag

SEQ ID NO: 67 - DNA, 474 bp, Homo sapiens
  modified_base (365)..(365): a, c, g, t, unknown or other
tactacaaaagccgtgacctcagcctgcttatactactgccagaagacattaatgggctggaacagctggaaaaggccatcacctatgagaagctgaatgagtggaccagtgcagacatgatggagttgtatgaagtgcagctacaccttcccaagttcaagctggaagacagttatgatctcaagtcaaccctgagcagtatggggatgagtgatgccttcagccaaagcaaagctgatttctcaggaatgtcttcagcaagaaacctatttttgtccaatgttttccataaggcttttgtggaaataaatgaacaaggtactgaagctgcagctggcagtgggagtgagatagatatacgaantagagtcccatccattgaattcaatgcaaatcacccattcctcttcttcatcaggcacaataanaaccaacaccattcttttttatggaagattatgctccccctaatc

SEQ ID NO: 68 - DNA, 287 bp, Homo sapiens
gattctgtggtagactcagtgctttcagagtccagagcttgacttgggttagtggccttaatgaagtgctaaatttgctctttaccgcgagactgatcagaagaagcaaaaggggaaagggggctagaggtccactcgcaccttttacatcagacaagaggaggactgtgccagaaatctgtgcatgaaacaccatctgctcttcatgcagggaggggtcaaccgtgtgaacgtgcagagattactcgagccttctttgccaaaaatatgcattcttcccagctgta

SEQ ID NO: 69 - DNA, 545 bp, Homo sapiens
taatcacatcacttccatggcatggatgttcacatacagactcttaaccctggtttaccaggacctctaggagtggatccaatctatatctttacagttgtatagtatatgatatctcttttatttcactcaatttatattttcatcattgactacatatttcttatacacaacacacaatttatgaattttttctcaagatcattctgagagttgccccaccctacctgccttttatagtacgcccacctcaggcagacacagagcacaatgctggggttctcttcacactatcactgccccaaattgtctttctaaatttcaacttcaatgtcatcttctccatgaagaccactgaatgaacaccttttcatccagccttaatttcttgctccataactactctatcccacgatgcagtattgtatcattaattattagtgtgcttgtgacctccttatgtattctcaattacctgtatttgtgcaataaattggaataatgtaacttgatttcttatctgtgtttgtgttggcatgcaagat

SEQ ID NO: 70 - DNA, 420 bp, Homo sapiens
aagacacttcttccaaaccttgaatttgttgtttttagaaaacgaatgcatttaaaaatattttctatgtgagaattttttagatgtgtgtttacttcatgtttacaaataactgtttgctttttaatgcagtactttgaaatatatcagccaaaaccataacttacaataatttcttaggtattctgaataaaattccatttcttttggatatgctttaccattcttaggtttctgtggaacaaaaatatttgtagcattttgtgtaaatacaagctttcatttttattttttccaattgctattgcccaagaattgctttccatgcacatattgtaaaaattccgctttgtgccacaggtcatgattgtggatgagtttactcttaacttcaaagggactatttgtattgtatgttgc

SEQ ID NO: 71 - DNA, 534 bp, Homo sapiens
  modified_base (486)..(493): a, c, g, t, unknown or other
agtattgacaactgcacatgaaagttttgcaaagggaaacaggctaaatgcaccaagaaagcttcttcagagtgaagaatcttaatgcttgtaatttaaacatttgttcctggagttttgatttggtggatgtgatggttggttttatttgtcagtttggttgggctatagcacacagttatttaatcaaacagtaatctaggtgtggctgtgaaggtattttgtagatgtgattaacatctacaatcagttgactttaagtgaaagagattacttaaataatttgggtgagctgcacctgattagttgaaaggcctcaagaacaaacactgcagtttcctggaaaagaagaaactttgcctcaagactatagccatcgactcctgcctgagtttccagcctgctagtctgccctatggatttgaagtttgccaaccccaacaattgtgtgaattaatttctaaaaataaagctatatacagccannnnnnnntatttgtgggggatttgtttcaggatctctacagataccaa

SEQ ID NO: 72 - DNA, 478 bp, Homo sapiens
ccctagaggggcaccttttcatggtctctgcacccagtgaacacattttactctagaggcatcacctgggaccttactcctctttccttccttcctcctttcctatcttccttcctctctctcttcctctttcttcattcagatctatatggcaaatagccacaattatataaatcatttcaagactagaatagggggatataatacatattactccacaccttttatgaatcaaatatgatttttttgttgttgttaagacagagtctcactttgacacccaggctggagtgcagtggtgccatcaccacggctcactgcagcctcagcgtcctgggctcaaatgatcctcccacctcagcctcctgagtagctgggactacaggctcatgccatcatgcccagctaatatttttttattttcgtggagacggggcctcactatgttgcctaggctggaaataggattttgaaccca

SEQ ID NO: 73 - DNA, 19 bp, Artificial Sequence
  source: /note=“Description of Artificial Sequence Synthetic primer”
ggatgctgagcggattctg

SEQ ID NO: 74 - DNA, 20 bp, Artificial Sequence
  source: /note=“Description of Artificial Sequence Synthetic primer”
ccctcgcgaaaaagtttctt

SEQ ID NO: 75 - DNA, 21 bp, Artificial Sequence
  source: /note=“Description of Artificial Sequence Synthetic primer”
aaggtctcagctgggcagttt

SEQ ID NO: 76 - DNA, 19 bp, Artificial Sequence
  source: /note=“Description of Artificial Sequence Synthetic primer”
aaactgggccacctcgatt

SEQ ID NO: 77 - DNA, 24 bp, Artificial Sequence
  source: /note=“Description of Artificial Sequence Synthetic probe”
ccagcttgcatgtccgagacacca

SEQ ID NO: 78 - DNA, 20 bp, Artificial Sequence
  source: /note=“Description of Artificial Sequence Synthetic primer”
gggtctcacctcccaactgc

SEQ ID NO: 79 - DNA, 22 bp, Artificial Sequence
  source: /note=“Description of Artificial Sequence Synthetic primer”
tgtctgttacggtcaactcggt

SEQ ID NO: 80 - DNA, 21 bp, Artificial Sequence
  source: /note=“Description of Artificial Sequence Synthetic primer”
gcttccccctctgttcttcct

SEQ ID NO: 81 - DNA, 23 bp, Artificial Sequence
  source: /note=“Description of Artificial Sequence Synthetic primer”
gctctgtgaggctgttcaaagtt

SEQ ID NO: 82 - DNA, 26 bp, Artificial Sequence
  source: /note=“Description of Artificial Sequence Synthetic probe”
tccacggacacaagtgcgatatcacc

SEQ ID NO: 83 - DNA, 20 bp, Artificial Sequence
  source: /note=“Description of Artificial Sequence Synthetic primer”
gccatgaggatgcttctgca

SEQ ID NO: 84 - DNA, 26 bp, Artificial Sequence
  source: /note=“Description of Artificial Sequence Synthetic primer”
gaatcctcagagtctcattggctatc

SEQ ID NO: 85 - DNA, 20 bp, Artificial Sequence
  source: /note=“Description of Artificial Sequence Synthetic primer”
agctgcctacgtgtatgcca

SEQ ID NO: 86 - DNA, 21 bp, Artificial Sequence
  source: /note=“Description of Artificial Sequence Synthetic primer”
gtgccaaggtctctttcacca

SEQ ID NO: 87 - DNA, 25 bp, Artificial Sequence
  source: /note=“Description of Artificial Sequence Synthetic probe”
ccccacagaaattcccacaagtgca

SEQ ID NO: 88 - DNA, 26 bp, Artificial Sequence
  source: /note=“Description of Artificial Sequence Synthetic primer”
actcctctacctccatcaataactcc

SEQ ID NO: 89 - DNA, 23 bp, Artificial Sequence
  source: /note=“Description of Artificial Sequence Synthetic primer”
tggctctgcaagagatgttagct

SEQ ID NO: 90 - DNA, 20 bp, Artificial Sequence
  source: /note=“Description of Artificial Sequence Synthetic primer”
gctggctggattctggaaaa

SEQ ID NO: 91 - DNA, 22 bp, Artificial Sequence
  source: /note=“Description of Artificial Sequence Synthetic primer”
tggctctgcaagagatgttagc

SEQ ID NO: 92 - DNA, 29 bp, Artificial Sequence
  source: /note=“Description of Artificial Sequence Synthetic probe”
tctccaatcaattctgtgtctccacctgg

SEQ ID NO: 93 - DNA, 18 bp, Artificial Sequence
  source: /note=“Description of Artificial Sequence Synthetic primer”
tgtggcgggaaagacagc

SEQ ID NO: 94 - DNA, 23 bp, Artificial Sequence
  source: /note=“Description of Artificial Sequence Synthetic primer”
ccttcctatggcttagcttcagc

SEQ ID NO: 95 - DNA, 20 bp, Artificial Sequence
  source: /note=“Description of Artificial Sequence Synthetic primer”
cgtgttgtcaccgagaacgt

SEQ ID NO: 96 - DNA, 20 bp, Artificial Sequence
  source: /note=“Description of Artificial Sequence Synthetic primer”
atcttgatggccttggagca

SEQ ID NO: 97 - DNA, 20 bp, Artificial Sequence
  source: /note=“Description of Artificial Sequence Synthetic probe”
ctgcggcaccacagggacca

SEQ ID NO: 98 - DNA, 19 bp, Artificial Sequence
  source: /note=“Description of Artificial Sequence Synthetic primer”
ttgaggacccctgctccct

SEQ ID NO: 99 - DNA, 20 bp, Artificial Sequence
  source: /note=“Description of Artificial Sequence Synthetic primer”
aggcgtgcacataggaggac

SEQ ID NO: 100 - DNA, 20 bp, Artificial Sequence
  source: /note=“Description of Artificial Sequence Synthetic primer”
cgatcccaacagtgccttct

SEQ ID NO: 101 - DNA, 18 bp, Artificial Sequence
  source: /note=“Description of Artificial Sequence Synthetic primer”
cctcgctccgctcacagt

SEQ ID NO: 102 - DNA, 23 bp, Artificial Sequence
  source: /note=“Description of Artificial Sequence Synthetic probe”
caaccccaagcccttccactcga

SEQ ID NO: 103 - DNA, 19 bp, Artificial Sequence
  source: /note=“Description of Artificial Sequence Synthetic primer”
ccaaccaccaaggatgcaa

SEQ ID NO: 104 - DNA, 18 bp, Artificial Sequence
  source: /note=“Description of Artificial Sequence Synthetic primer”
tctgcccagctgccaagt

SEQ ID NO: 105 - DNA, 19 bp, Artificial Sequence
  source: /note=“Description of Artificial Sequence Synthetic primer”
ccaaccaccaaggatgcaa

SEQ ID NO: 106 - DNA, 21 bp, Artificial Sequence
  source: /note=“Description of Artificial Sequence Synthetic primer”
ggagagaagcctggtggaagt

SEQ ID NO: 107 - DNA, 25 bp, Artificial Sequence
  source: /note=“Description of Artificial Sequence Synthetic probe”
cagtgtcgccatcactgtctccagc

SEQ ID NO: 108 - DNA, 20 bp, Artificial Sequence
  source: /note=“Description of Artificial Sequence Synthetic primer”
acgtccaccagaccatcacc

SEQ ID NO: 109 - DNA, 20 bp, Artificial Sequence
  source: /note=“Description of Artificial Sequence Synthetic primer”
gaatctcacgtgtgccacca

SEQ ID NO: 110 - DNA, 19 bp, Artificial Sequence
  source: /note=“Description of Artificial Sequence Synthetic primer”
attgcaatgtaccgccagc

SEQ ID NO: 111 - DNA, 20 bp, Artificial Sequence
  source: /note=“Description of Artificial Sequence Synthetic primer”
gaatctcacgtgtgccacca

SEQ ID NO: 112 - DNA, 26 bp, Artificial Sequence
  source: /note=“Description of Artificial Sequence Synthetic probe”
ctgctgtgcaccccattttcaagctg

SEQ ID NO: 113 - DNA, 22 bp, Artificial Sequence
  source: /note=“Description of Artificial Sequence Synthetic primer”
cataaagtggagcacgaaagca

SEQ ID NO: 114 - DNA, 25 bp, Artificial Sequence
  source: /note=“Description of Artificial Sequence Synthetic primer”
ggtacgcatctacacagttctggtt

SEQ ID NO: 115 - DNA, 20 bp, Artificial Sequence
  source: /note=“Description of Artificial Sequence Synthetic primer”
cagaatgggaggagcttcca

SEQ ID NO: 116 - DNA, 23 bp, Artificial Sequence
  source: /note=“Description of Artificial Sequence Synthetic primer”
cacagttctggttggcagtgtag

SEQ ID NO: 117 - DNA, 23 bp, Artificial Sequence
  source: /note=“Description of Artificial Sequence Synthetic probe”
ccggaacacttgcctttgagcgg

SEQ ID NO: 118 - DNA, 21 bp, Artificial Sequence
  source: /note=“Description of Artificial Sequence Synthetic primer”
caaggtctgtgggaaaagcaa

SEQ ID NO: 119 - DNA, 20 bp, Artificial Sequence
  source: /note=“Description of Artificial Sequence Synthetic primer”
tggccagagatgcttccaat

SEQ ID NO: 120 - DNA, 24 bp, Artificial Sequence
  source: /note=“Description of Artificial Sequence Synthetic primer”
gcagaagtcaagaagaacggaaga

SEQ ID NO: 121 - DNA, 20 bp, Artificial Sequence
  source: /note=“Description of Artificial Sequence Synthetic primer”
tgcttccaattgccaaactg

SEQ ID NO: 122 - DNA, 20 bp, Artificial Sequence
  source: /note=“Description of Artificial Sequence Synthetic probe”
tctccgcccagcaccaggct

SEQ ID NO: 123 - DNA, 21 bp, Artificial Sequence
  source: /note=“Description of Artificial Sequence Synthetic primer”
acttaaagcccgcctgacaga

SEQ ID NO: 124 - DNA, 22 bp, Artificial Sequence
  source: /note=“Description of Artificial Sequence Synthetic primer”
gctacttcttgccccctttgaa

SEQ ID NO: 125 - DNA, 18 bp, Artificial Sequence
  source: /note=“Description of Artificial Sequence Synthetic primer”
ccacggccacatttggtt

SEQ ID NO: 126 - DNA, 19 bp, Artificial Sequence
  source: /note=“Description of Artificial Sequence Synthetic primer”
agggaagcggttgctcatc

SEQ ID NO: 127 - DNA, 28 bp, Artificial Sequence
  source: /note=“Description of Artificial Sequence Synthetic probe”
agaaaccctctgtcattcgctcccacat

SEQ ID NO: 128 - DNA, 23 bp, Artificial Sequence
  source: /note=“Description of Artificial Sequence Synthetic primer”
ctccgcagtcacctaatcactct

SEQ ID NO: 129 - DNA, 25 bp, Artificial Sequence
  source: /note=“Description of Artificial Sequence Synthetic primer”
ggctcaatgggtaccacatctatct

SEQ ID NO: 130 - DNA, 23 bp, Artificial Sequence
  source: /note=“Description of Artificial Sequence Synthetic primer”
ttcctgttccattcagagacgat

SEQ ID NO: 131 - DNA, 23 bp, Artificial Sequence
  source: /note=“Description of Artificial Sequence Synthetic primer”
agattctgaaggcttgcatcttg

SEQ ID NO: 132 - DNA, 24 bp, Artificial Sequence
  source: /note=“Description of Artificial Sequence Synthetic probe”
tgccgaccctctgggagaaaatcc

SEQ ID NO: 133 - DNA, 22 bp, Artificial Sequence
  source: /note=“Description of Artificial Sequence Synthetic primer”
attcaaggatcttgctgccttt

SEQ ID NO: 134 - DNA, 18 bp, Artificial Sequence
  source: /note=“Description of Artificial Sequence Synthetic primer”
tgcagtcacgggatgcat

SEQ ID NO: 135 - DNA, 21 bp, Artificial Sequence
  source: /note=“Description of Artificial Sequence Synthetic primer”
caaggatcttgctgcctttga

SEQ ID NO: 136 - DNA, 21 bp, Artificial Sequence
  source: /note=“Description of Artificial Sequence Synthetic primer”
tgcttgctttgtgctcttggt

SEQ ID NO: 137 - DNA, 27 bp, Artificial Sequence
  source: /note=“Description of Artificial Sequence Synthetic probe”
aaatcccatgatcaagctgtccgaacc

SEQ ID NO: 138 - DNA, 19 bp, Artificial Sequence
  source: /note=“Description of Artificial Sequence Synthetic primer”
caccacaccgacggtacca

SEQ ID NO: 139 - DNA, 15 bp, Artificial Sequence
  source: /note=“Description of Artificial Sequence Synthetic primer”
tgcgcgccgagatca

SEQ ID NO: 140 - DNA, 23 bp, Artificial Sequence
  source: /note=“Description of Artificial Sequence Synthetic primer”
ccatgaaggacgaggtagctcta

SEQ ID NO: 141 - DNA, 15 bp, Artificial Sequence
  source: /note=“Description of Artificial Sequence Synthetic primer”
tgcgcgccgagatca

SEQ ID NO: 142 - DNA, 26 bp, Artificial Sequence
  source: /note=“Description of Artificial Sequence Synthetic probe”
cctgggagtcctgctgcaagcctact

SEQ ID NO: 143 - DNA, 21 bp, Artificial Sequence
  source: /note=“Description of Artificial Sequence Synthetic primer”
cgctactaggcaatgccaatg

SEQ ID NO: 144 - DNA, 23 bp, Artificial Sequence
  source: /note=“Description of Artificial Sequence Synthetic primer”
gcaatctgcgtaccacttgtttt

SEQ ID NO: 145 - DNA, 21 bp, Artificial Sequence
  source: /note=“Description of Artificial Sequence Synthetic primer”
cgctactaggcaatgccaatg

SEQ ID NO: 146 - DNA, 23 bp, Artificial Sequence
  source: /note=“Description of Artificial Sequence Synthetic primer”
gcaatctgcgtaccacttgtttt

SEQ ID NO: 147 - DNA, 26 bp, Artificial Sequence
  source: /note=“Description of Artificial Sequence Synthetic probe”
agcaacctgtgcattcccgttcaagt

SEQ ID NO: 148 - DNA, 22 bp, Artificial Sequence
  source: /note=“Description of Artificial Sequence Synthetic primer”
gggagcactgctattctttcca

SEQ ID NO: 149 - DNA, 24 bp, Artificial Sequence
  source: /note=“Description of Artificial Sequence Synthetic primer”
caaacacattctccatctcatcca

SEQ ID NO: 150 - DNA, 24 bp, Artificial Sequence
  source: /note=“Description of Artificial Sequence Synthetic primer”
gagcactgctattctttccacatg

SEQ ID NO: 151 - DNA, 24 bp, Artificial Sequence
  source: /note=“Description of Artificial Sequence Synthetic primer”
tctccatctcatccaggatagaca

SEQ ID NO: 152 - DNA, 20 bp, Artificial Sequence
  source: /note=“Description of Artificial Sequence Synthetic probe”
ccacccgctctctggcagcg

SEQ ID NO: 153 - DNA, 20 bp, Artificial Sequence
  source: /note=“Description of Artificial Sequence Synthetic primer”
gcatggctcgcctacagact

SEQ ID NO: 154 - DNA, 23 bp, Artificial Sequence
  source: /note=“Description of Artificial Sequence Synthetic primer”
cagacggtaacggacgtaatcac

SEQ ID NO: 155 - DNA, 18 bp, Artificial Sequence
  source: /note=“Description of Artificial Sequence Synthetic primer”
tggcgcttcaagcaactg

SEQ ID NO: 156 - DNA, 22 bp, Artificial Sequence
  source: /note=“Description of Artificial Sequence Synthetic primer”
cagacggtaacggacgtaatca

SEQ ID NO: 157 - DNA, 21 bp, Artificial Sequence
  source: /note=“Description of Artificial Sequence Synthetic probe”
aggcccctacggcgccaacat

SEQ ID NO: 158 - DNA, 23 bp, Artificial Sequence
  source: /note=“Description of Artificial Sequence Synthetic primer”
ctggtatgagcccatctatctgg

SEQ ID NO: 159 - DNA, 21 bp, Artificial Sequence
  source: /note=“Description of Artificial Sequence Synthetic primer”
ttggatgttcgtcctcctcac

SEQ ID NO: 160 - DNA, 20 bp, Artificial Sequence
  source: /note=“Description of Artificial Sequence Synthetic primer”
ggagaagggtgaccgactca

SEQ ID NO: 161 - DNA, 18 bp, Artificial Sequence
  source: /note=“Description of Artificial Sequence Synthetic primer”
tgcccagactcggcaaag

SEQ ID NO: 162 - DNA, 24 bp, Artificial Sequence
  source: /note=“Description of Artificial Sequence Synthetic probe”
cgctgagatcaatcggcccgacta

SEQ ID NO: 163 - DNA, 24 bp, Artificial Sequence
  source: /note=“Description of Artificial Sequence Synthetic primer”
ggctgtgacatcaatgctatcatc

SEQ ID NO: 164 - DNA, 26 bp, Artificial Sequence
  source: /note=“Description of Artificial Sequence Synthetic primer”
gtccagtgaggcacaaattagataag

SEQ ID NO: 165 - DNA, 28 bp, Artificial Sequence
  source: /note=“Description of Artificial Sequence Synthetic primer”
tctggaatggaattggacatagcccaag

SEQ ID NO: 166 - DNA, 24 bp, Artificial Sequence
  source: /note=“Description of Artificial Sequence Synthetic primer”
ccaaccccagcaaggttctttctg

SEQ ID NO: 167 - DNA, 26 bp, Artificial Sequence
  source: /note=“Description of Artificial Sequence Synthetic probe”
accctccatgatgtgcaagtgaaacc

SEQ ID NO: 168 - DNA, 23 bp, Artificial Sequence
  source: /note=“Description of Artificial Sequence Synthetic primer”
ggatgccatcgtttttgtaactg

SEQ ID NO: 169 - DNA, 21 bp, Artificial Sequence
  source: /note=“Description of Artificial Sequence Synthetic primer”
cctctcaaggctttgcaggta

SEQ ID NO: 170 - DNA, 18 bp, Artificial Sequence
  source: /note=“Description of Artificial Sequence Synthetic primer”
gggcagggccatctgttc

SEQ ID NO: 171 - DNA, 24 bp, Artificial Sequence
  source: /note=“Description of Artificial Sequence Synthetic primer”
tctcaaggctttgcaggtatttaa

SEQ ID NO: 172 - DNA, 29 bp, Artificial Sequence
  source: /note=“Description of Artificial Sequence Synthetic probe”
accccaacaacaagagagtgaagaatgca

SEQ ID NO: 173 - DNA, 20 bp, Artificial Sequence
  source: /note=“Description of Artificial Sequence Synthetic primer”
cctcctccttgtggctaccc

SEQ ID NO: 174 - DNA, 23 bp, Artificial Sequence
  source: /note=“Description of Artificial Sequence Synthetic primer”
caatctcttcagaagtgcaaggg

SEQ ID NO: 175 - DNA, 20 bp, Artificial Sequence
  source: /note=“Description of Artificial Sequence Synthetic primer”
tcctcctggaccacctcagt

SEQ ID NO: 176 - DNA, 22 bp, Artificial Sequence
  source: /note=“Description of Artificial Sequence Synthetic primer”
gaacattcctgggtctggagtg

SEQ ID NO: 177 - DNA, 21 bp, Artificial Sequence
  source: /note=“Description of Artificial Sequence Synthetic probe”
tggccagaaacctccccgtgg

SEQ ID NO: 178 - DNA, 24 bp, Artificial Sequence
  source: /note=“Description of Artificial Sequence Synthetic primer”
gtaactgacttgaatgtccaacgc

SEQ ID NO: 179 - DNA, 22 bp, Artificial Sequence
  source: /note=“Description of Artificial Sequence Synthetic primer”
gacaaccattactgggatgctc

SEQ ID NO: 180 - DNA, 21 bp, Artificial Sequence
  source: /note=“Description of Artificial Sequence Synthetic primer”
ccaacgcaaagcaatacatga

SEQ ID NO: 181 - DNA, 22 bp, Artificial Sequence
  source: /note=“Description of Artificial Sequence Synthetic primer”
ttttcgcttccctgttttagct

SEQ ID NO: 182 - DNA, 25 bp, Artificial Sequence
  source: /note=“Description of Artificial Sequence Synthetic probe”
tccaagtgatggctgaactgtcgcc

SEQ ID NO: 183 - DNA, 20 bp, Artificial Sequence
  source: /note=“Description of Artificial Sequence Synthetic primer”
gttgcctggtcctcctgact

SEQ ID NO: 184 - DNA, 22 bp, Artificial Sequence
  source: /note=“Description of Artificial Sequence Synthetic primer”
tgtccagctgatccttcatttg

SEQ ID NO: 185 - DNA, 21 bp, Artificial Sequence
  source: /note=“Description of Artificial Sequence Synthetic primer”
tgagaacagctgcacccactt

SEQ ID NO: 186 - DNA, 20 bp, Artificial Sequence
  source: /note=“Description of Artificial Sequence Synthetic primer”
gctgaaggcatctcggagat

SEQ ID NO: 187 - DNA, 25 bp, Artificial Sequence
  source: /note=“Description of Artificial Sequence Synthetic probe”
caggcaacctgcctaacatgcttcg

SEQ ID NO: 188 - DNA, 21 bp, Artificial Sequence
  source: /note=“Description of Artificial Sequence Synthetic primer”
actgctactgctgctgagcct

SEQ ID NO: 189 - DNA, 22 bp, Artificial Sequence
  source: /note=“Description of Artificial Sequence Synthetic primer”
ggtgaggtggatcggttgtagt

SEQ ID NO: 190 - DNA, 20 bp, Artificial Sequence
  source: /note=“Description of Artificial Sequence Synthetic primer”
caatcccacgaaatccagga

SEQ ID NO: 191 - DNA, 22 bp, Artificial Sequence
  source: /note=“Description of Artificial Sequence Synthetic primer”
ttcaggttgaccatcacagtcc

SEQ ID NO: 192 - DNA, 28 bp, Artificial Sequence
  source: /note=“Description of Artificial Sequence Synthetic probe”
cccaaattctgaggacaagaacttcccc

SEQ ID NO: 193 - protein, 118 aa, Artificial Sequence [sequence omitted]

SEQ ID NO: 194 - protein, 111 aa, Artificial Sequence [sequence omitted]

SEQ ID NO: 195 - protein, 15 aa, Artificial Sequence
  source: /note=“Description of Artificial Sequence Synthetic peptide”
RASKSVDSYGNSFMH

SEQ ID NO: 196 - protein, 7 aa, Artificial Sequence
  source: /note=“Description of Artificial Sequence Synthetic peptide”
LASNLES

SEQ ID NO: 197 - protein, 9 aa, Artificial Sequence
  source: /note=“Description of Artificial Sequence Synthetic peptide”
QQNNEDPRT

SEQ ID NO: 198 - protein, 5 aa, Artificial Sequence
  source: /note=“Description of Artificial Sequence Synthetic peptide”
AYSVN

SEQ ID NO: 199 - protein, 16 aa, Artificial Sequence
  source: /note=“Description of Artificial Sequence Synthetic peptide”
MIWGDGKIVYNSALKS

SEQ ID NO: 200 - protein, 10 aa, Artificial Sequence
  source: /note=“Description of Artificial Sequence Synthetic peptide”
DGYYPYAMDN

SEQ ID NO: 201 - protein, 16 aa, Artificial Sequence
  source: /note=“Description of Artificial Sequence Synthetic peptide”
RSSQSPVHSNGNTYLH

SEQ ID NO: 202 - protein, 7 aa, Artificial Sequence
  source: /note=“Description of Artificial Sequence Synthetic peptide”
KVSNRFS

SEQ ID NO: 203 - protein, 9 aa, Artificial Sequence
  source: /note=“Description of Artificial Sequence Synthetic peptide”
SQSTHIPWT

SEQ ID NO: 204 - protein, 5 aa, Artificial Sequence
  source: /note=“Description of Artificial Sequence Synthetic peptide”
SYWMH

SEQ ID NO: 205 - protein, 17 aa, Artificial Sequence
  source: /note=“Description of Artificial Sequence Synthetic peptide”
EIDPSNGRTNYNEKFKS

SEQ ID NO: 206 - protein, 9 aa, Artificial Sequence
  source: /note=“Description of Artificial Sequence Synthetic peptide”
ERSPRYFDV

SEQ ID NO: 207 - protein, 16 aa, Artificial Sequence
  source: /note=“Description of Artificial Sequence Synthetic peptide”
RSSQSIVHGNGNTYLE

SEQ ID NO: 208 - protein, 7 aa, Artificial Sequence
  source: /note=“Description of Artificial Sequence Synthetic peptide”
RVSNRFS

SEQ ID NO: 209 - protein, 9 aa, Artificial Sequence
  source: /note=“Description of Artificial Sequence Synthetic peptide”
FQGSHVPYT

SEQ ID NO: 210 - protein, 5 aa, Artificial Sequence
  source: /note=“Description of Artificial Sequence Synthetic peptide”
SYWLN

SEQ ID NO: 211 - protein, 17 aa, Artificial Sequence
  source: /note=“Description of Artificial Sequence Synthetic peptide”
MIDPSDSETHYNQVFKD

SEQ ID NO: 212 - protein, 14 aa, Artificial Sequence
  source: /note=“Description of Artificial Sequence Synthetic peptide”
GRGNFYGGSHAMEY

SEQ ID NO: 213 - protein, 114 aa, Artificial Sequence
  source: /note=“Description of Artificial Sequence Synthetic E25 light chain variable sequence”
DIQLTQSPSSLSASVGDRVTITCRASQSVDYDGDSYMNWYQQKPGKAPKLLIYAASYLESGVPSRFSGSGSGTDFTLTISSLQPEDFATYYCQQSHEDPYTFGQGTKVEIKRTV

SEQ ID NO: 214 - protein, 114 aa, Artificial Sequence
  source: /note=“Description of Artificial Sequence Synthetic E25 heavy chain variable sequence”
EVQLVESGGGLVQPGGSLRLSCAVSGYSITSGYSWNWIRQAPGKGLEWVASITYDGSTNYNPSVKGRITISRDDSKNTFYLQMNSLRAEDTAVYYCARGSHYFGHWHFAVWGQG

SEQ ID NO: 215 - protein, 5 aa, Artificial Sequence
  source: /note=“Description of Artificial Sequence Synthetic peptide”
SYTMH

SEQ ID NO: 216 - protein, 5 aa, Artificial Sequence
  source: /note=“Description of Artificial Sequence Synthetic peptide”
SYAMS

SEQ ID NO: 217 - protein, 5 aa, Artificial Sequence
  source: /note=“Description of Artificial Sequence Synthetic peptide”
NFGMH

SEQ ID NO: 218 - protein, 5 aa, Artificial Sequence
  source: /note=“Description of Artificial Sequence Synthetic peptide”
NYGMH

SEQ ID NO: 219 - protein, 17 aa, Artificial Sequence
  source: /note=“Description of Artificial Sequence Synthetic peptide”
IISGSGGFTYYADSVKG

SEQ ID NO: 220 - protein, 17 aa, Artificial Sequence
  source: /note=“Description of Artificial Sequence Synthetic peptide”
AIWYDGHDKYYSYYVKG

SEQ ID NO: 221 - protein, 17 aa, Artificial Sequence
  source: /note=“Description of Artificial Sequence Synthetic peptide”
AIWYDGHDKYYAYYVKG

SEQ ID NO: 222 - protein, 17 aa, Artificial Sequence
  source: /note=“Description of Artificial Sequence Synthetic peptide”
VIWYDGSNKYYVDSVKG

SEQ ID NO: 223 - protein, 17 aa, Artificial Sequence
  source: /note=“Description of Artificial Sequence Synthetic peptide”
VIWNDGSNKYYVDSVKG

SEQ ID NO: 224 - protein, 11 aa, Artificial Sequence
  source: /note=“Description of Artificial Sequence Synthetic peptide”
DSSSWYRYFDY

SEQ ID NO: 225 - protein, 11 aa, Artificial Sequence
  source: /note=“Description of Artificial Sequence Synthetic peptide”
DRLVAPGTFDY

SEQ ID NO: 226 - protein, 7 aa, Artificial Sequence
  source: /note=“Description of Artificial Sequence Synthetic peptide”
KNWSFDF

SEQ ID NO: 227 - protein, 12 aa, Artificial Sequence
  source: /note=“Description of Artificial Sequence Synthetic peptide”
DRMGIYYYGMDV

SEQ ID NO: 228 - protein, 11 aa, Artificial Sequence
  source: /note=“Description of Artificial Sequence Synthetic peptide”
RASQGISSWLA

SEQ ID NO: 229 - protein, 12 aa, Artificial Sequence
  source: /note=“Description of Artificial Sequence Synthetic peptide”
RASQSVSSSYLA

SEQ ID NO: 230 - protein, 12 aa, Artificial Sequence
  source: /note=“Description of Artificial Sequence Synthetic peptide”
RASQSVSSNYLA

SEQ ID NO: 231 - protein, 11 aa, Artificial Sequence
  source: /note=“Description of Artificial Sequence Synthetic peptide”
RASQGVSRYLA

SEQ ID NO: 232 - protein, 11 aa, Artificial Sequence
  source: /note=“Description of Artificial Sequence Synthetic peptide”
RASQSVSSYLA

SEQ ID NO: 233 - protein, 7 aa, Artificial Sequence
  source: /note=“Description of Artificial Sequence Synthetic peptide”
GASSRAT

SEQ ID NO: 234 - protein, 7 aa, Artificial Sequence
  source: /note=“Description of Artificial Sequence Synthetic peptide”
AASSLQS

SEQ ID NO: 235 - protein, 7 aa, Artificial Sequence
  source: /note=“Description of Artificial Sequence Synthetic peptide”
MPPVWKV

SEQ ID NO: 236 - protein, 7 aa, Artificial Sequence
  source: /note=“Description of Artificial Sequence Synthetic peptide”
DASNRAT

SEQ ID NO: 237 - protein, 7 aa, Artificial Sequence
  source: /note=“Description of Artificial Sequence Synthetic peptide”
LHPLCKV

SEQ ID NO: 238 - protein, 8 aa, Artificial Sequence
  source: /note=“Description of Artificial Sequence Synthetic peptide”
NSLIVTLT

SEQ ID NO: 239 - protein, 9 aa, Artificial Sequence
  source: /note=“Description of Artificial Sequence Synthetic peptide”
QQYNSYPYT

SEQ ID NO: 240 - protein, 8 aa, Artificial Sequence
  source: /note=“Description of Artificial Sequence Synthetic peptide”
QQYGSSFT

SEQ ID NO: 241 - protein, 9 aa, Artificial Sequence
  source: /note=“Description of Artificial Sequence Synthetic peptide”
QQRSNWQYT

SEQ ID NO: 242 - protein, 7 aa, Artificial Sequence
  source: /note=“Description of Artificial Sequence Synthetic peptide”
QQRSNWT

SEQ ID NO: 243 - protein, 8 aa, Artificial Sequence
  source: /note=“Description of Artificial Sequence Synthetic peptide”
NSIIVSLT

SEQ ID NO: 244 - protein, 16 aa, Artificial Sequence
  source: /note=“Description of Artificial Sequence Synthetic peptide”
RSSQSLVHNNANTYLH

SEQ ID NO: 245 - protein, 7 aa, Artificial Sequence
  source: /note=“Description of Artificial Sequence Synthetic peptide”
KVSNRFS

SEQ ID NO: 246 - protein, 9 aa, Artificial Sequence
  source: /note=“Description of Artificial Sequence Synthetic peptide”
SQNTLVPWT

SEQ ID NO: 247 - protein, 10 aa, Artificial Sequence
  source: /note=“Description of Artificial Sequence Synthetic peptide”
GFTFSDYGIA

SEQ ID NO: 248 - protein, 18 aa, Artificial Sequence
  source: /note=“Description of Artificial Sequence Synthetic peptide”
AFISDLAYTIYYADTVTG

SEQ ID NO: 249 - protein, 10 aa, Artificial Sequence
  source: /note=“Description of Artificial Sequence Synthetic peptide”
ARDNWDAMDY

SEQ ID NO: 250 - protein, 117 aa, Artificial Sequence
  source: /note=“Description of Artificial Sequence Synthetic Humanized 47H4 V.5 heavy chain variable sequence”
EVQLVESGGGLVQPGGSLRLSCAASGFTFSDYGIAWVRQAPGKGLEWVAFISDLAYTIYYADTVTGRFTISRDNSKNTLYLQMNSLRAEDTAVYYCARDNWDAMDYWGQGTLVTVSS

SEQ ID NO: 251 - protein, 113 aa, Artificial Sequence
  source: /note=“Description of Artificial Sequence Synthetic Humanized 47H4 V.24-6 light chain variable sequence”
DIQMTQSPSSLSASVGDRVTITCRSSQSLVHNNANTYLHWYQQKPGKAPKLLIYKVSNRFSGVPSRFSGSGSGTDFTLTISSLQPEDFATYYCSQNTLVPWTFGQGTKVEIKR

SEQ ID NO: 252 - protein, 117 aa, Artificial Sequence
  source: /note=“Description of Artificial Sequence Synthetic Humanized 47H4 V.1,2 heavy chain variable sequence”
EVQLVESGGGLVQPGGSLRLSCAASGFTFSDYGMAWVRQAPGKGLEWVAFISDLAYTIYYADTVTGRFTISRDNSKNTLYLQMNSLRAEDTAVYYCARDNWDAMDYWGQGTLVTVSS

SEQ ID NO: 253 - protein, 113 aa, Artificial Sequence
  source: /note=“Description of Artificial Sequence Synthetic Humanized 47H4 V.1,3 light chain variable sequence”
DIQMTQSPSSLSASVGDRVTITCRSSQSLVHNNGNTYLHWYQQKPGKAPKLLIYKVSNRFSGVPSRFSGSGSGTDFTLTISSLQPEDFATYYCSQNTLVPWTFGQGTKVEIKR

SEQ ID NO: 254 - protein, 11 aa, Artificial Sequence
  source: /note=“Description of Artificial Sequence Synthetic peptide”
RSSQDISNSLN

SEQ ID NO: 255 - protein, 7 aa, Artificial Sequence
  source: /note=“Description of Artificial Sequence Synthetic peptide”
STSRLHS

SEQ ID NO: 256 - protein, 9 aa, Artificial Sequence
  source: /note=“Description of Artificial Sequence Synthetic peptide”
QQGHTLPWT

SEQ ID NO: 257 - protein, 10 aa, Artificial Sequence
  source: /note=“Description of Artificial Sequence Synthetic peptide”
GYTFTDYYMM

SEQ ID NO: 258 - protein, 19 aa, Artificial Sequence
  source: /note=“Description of Artificial Sequence Synthetic peptide”
GDNIDPNNYDTSYNQKFKG

SEQ ID NO: 259 - protein, 5 aa, Artificial Sequence
  source: /note=“Description of Artificial Sequence Synthetic peptide”
ASKAY

SEQ ID NO: 260 - protein, 11 aa, Artificial Sequence
  source: /note=“Description of Artificial Sequence Synthetic peptide”
RSSQDISNALN

SEQ ID NO: 261 - protein, 10 aa, Artificial Sequence
  source: /note=“Description of Artificial Sequence Synthetic peptide”
GYTFTDYYIM